(19) United States
(12) Patent Application Publication
(10) Pub. No.: US 2018/0200291 A1
(43) Pub. Date: Jul. 19, 2018

(54) COMPOSITIONS COMPRISING A ZEOLITE AND USE THEREOF FOR THE TREATMENT OF TRIMETHYLAMINURIA

(71) Applicant: NeuroScience, Inc., Osceola, WI (US)

(72) Inventors: Gottfried H. Kellermann, Osceola, WI (US); Gustav F. Jirikowski, Osceola, WI (US)

(21) Appl. No.: 15/873,596

(22) Filed: Jan. 17, 2018

Related U.S. Application Data
(60) Provisional application No. 62/447,866, filed on Jan. 18, 2017.

(51) Int. Cl. A61K 33/06; A61K 33/44; A61K 31/525; A61K 31/355; A61K 31/7016; A61K 31/437; A61K 31/4164; A61K 31/7036; A61K 35/744; A61K 35/745; A61K 35/747; A61K 31/198; A61K 31/555

(52) U.S. Cl. CPC A61K 33/06; A61K 33/44; A61K 31/525; A61K 31/355; A61K 31/7016; A61K 31/437; A61K 31/555; A61K 31/7036; A61K 35/744; A61K 35/745; A61K 35/747; A61K 31/198; A61K 31/4164

(57) ABSTRACT

Provided herein are methods of treating trimethylaminuria in a subject by administering a composition including a zeolite.

### CROSS-REFERENCE TO RELATED APPLICATION

This application claims priority to U.S. Provisional Patent Application Ser. No. 62/447,866, filed Jan. 18, 2017, which is herein incorporated by reference in its entirety.

### TECHNICAL FIELD

This document relates to the field of compositions. For example, this document relates to compositions comprising zeolites and methods of treating trimethylaminuria.

### BACKGROUND

The compositions described herein may be used to reduce a level of trimethylamine in a subject in need thereof. Thus, the compositions described herein may be used to treat trimethylaminuria (e.g., primary trimethylaminuria and/or secondary trimethylaminuria) in a subject.

### SUMMARY

Provided herein are methods of treating trimethylaminuria (TMAU), decreasing a level of trimethylamine, a method of increasing a trimethylamine N-oxide ratio, and a method of decreasing a trimethylamine ratio in a subject in need thereof that include orally administering to the subject a therapeutically effective amount of a composition including a zeolite.

Provided herein are methods of treating trimethylaminuria (TMAU) in a subject in need thereof that include orally administering to the subject a therapeutically effective amount of a composition including a zeolite.

Also provided herein are methods of decreasing a level of trimethylamine in one or more of urine, sweat, reproductive fluid, saliva, blood, plasma, serum, and breath in a subject in need thereof that include orally administering to the subject a therapeutically effective amount of a composition including a zeolite.

Also provided are methods of decreasing a level of trimethylamine precursor in one or more of urine, sweat, reproductive fluid, saliva, blood, plasma, serum, and breath in a subject in need thereof that include orally administering to the subject a therapeutically effective amount of a composition including a zeolite.

Also provided are methods of increasing a trimethylamine N-oxide ratio in one or more of urine, sweat, reproductive fluid, saliva, blood, plasma, serum, and breath in a subject in need thereof that include orally administering to the subject a therapeutically effective amount of a composition including a zeolite, where the trimethylamine N-oxide ratio is Formula I:

[(level of trimethylamine N-oxide)/(level of trimethylamine N-oxide+level of trimethylamine)]×100%.

Also provided are methods of decreasing a trimethylamine ratio in one or more of urine, sweat, reproductive fluid, saliva, blood, serum, plasma, and breath in a subject in need thereof that include orally administering to the subject a therapeutically effective amount of a composition including a zeolite, where the trimethylamine ratio is Formula II:

[(level of trimethylamine)/(level of trimethylamine N-oxide+level of trimethylamine)]×100%.

In some embodiments of any of the methods described herein, the subject has been diagnosed as having primary TMAU (TMAU1). In some embodiments of any of the methods described herein, the subject has been determined to have a dysregulating mutation in a flavin-containing monooxygenase 3 (FMO3) gene.

In some embodiments of any of the methods described herein, the subject has been diagnosed as having secondary TMAU (TMAU2). In some embodiments of any of the methods described herein, the subject does not have a urinary tract infection, bacterial vaginosis, cervical cancer, or advanced liver or kidney disease. In some embodiments of any of the methods described herein, the subject has been identified with enzymatic defects associated with TMAU (TMAU2).

In some embodiments of any of the methods described herein, the composition is a zeolite suspension or a zeolite slurry comprising one or more solutions. In some embodiments of any of the methods described herein, the one or more solution includes an acidic solution, a neutral solution, a basic solution, an inorganic acid, an organic acid, an acid anhydride, an acidic salt, or a combination thereof. In some embodiments of any of the methods described herein, the one or more solution is a sodium aluminate solution, a sodium silicate solution, a sodium water-glass solution, a sodium aluminate solution, sulfuric acid, sulfurous acid, hydrochloric acid, hydrobromic acid, hypochlorous acid, chloric acid, boric acid, nitric acid, phosphoric acid, phosphorous acid, metaphosphoric acid, acetic acid, propionic acid, oxalic acid, citric acid, tartaric acid, succinic acid, maleic acid, malic acid, gluconic acid, itaconic acid, thioglycolic acid, ethylenediamine-tetraacetic acid, nitrilotriacetic acid, diglycolic acid, sulfoitaconic acid, trimellitic acid, pyromellitic acid, acetic anhydride, succinic anhydride, water, a sodium hydroxide solution, or a combination thereof.

In some embodiments of any of the methods described herein, about 0.1% w/w to about 25% w/w of the composition is represented by the zeolite. In some embodiments of any of the methods described herein, the one or more solution or combination thereof, the zeolite suspension, or the zeolite slurry has a pH of about 9.0 to about 13.0 (e.g., about 9.5 to about 12.0, or about 11.0 to about 12.7).

In some embodiments of any of the methods described herein, the zeolite has the following formula: M_{a/n} (AlO₂)ₐ(SiO₂)_{b}.wH₂O, where w is the number of water molecules per unit cell varying from 1 to 10; and a and b are total numbers of tetrahedra of Al and Si, respectively, where the ratio of b/a ranges from about 0.5 to about 5 (e.g., about 0.5 to about 3.0, about 0.5 to about 2.5, about 0.5 to about 2.0, or about 1).

In some embodiments of any of the methods described herein, the zeolite has a pore size of about 2.0 angstroms to about 5.5 angstroms (e.g., about 2.0 angstroms to about 5.0 angstroms, or about 2.0 angstroms to about 4.0 angstroms).

In some embodiments of any of the methods described herein, the composition is a pharmaceutical composition. In some embodiments of any of the methods described herein, the zeolite is a natural zeolite. In some embodiments of any of the methods described herein, the zeolite is a synthetic zeolite. In some embodiments of any of the methods described herein, the zeolite is selected from the group of a Type 3A zeolite, a Type 4A zeolite, a Type 5A zeolite, a Type 13X zeolite, and combinations thereof. In some embodiments of any of the methods described herein, the zeolite is a Type 4A zeolite.

In some embodiments of any of the methods described herein, the composition comprises about 100 mg to about 3,000 mg (e.g., about 500 mg to about 1,500 mg) of the zeolite. In some embodiments of any of the methods described herein, the zeolite is the only active ingredient in the composition.

In some embodiments of any of the methods described herein, the composition further includes one or more additional active ingredients selected from the group consisting of: riboflavin, a colonic acidifier, an ammonia reducer, an ammonia sequestering agent, an intestinal deodorizer, and a probiotic. In some embodiments of the methods described herein, the composition comprises about 1 mg to about 2,000 mg (e.g., about 10 mg to about 1,000 mg) of riboflavin. In some embodiments of any of the methods described herein, the composition comprises a colonic acidifier (e.g., lactulose). In some embodiments of any of the methods described herein, the composition includes about 1 g to about 15 g (e.g., about 5 g to about 15 g) of lactulose. In some embodiments of any of the methods described herein, the composition comprises an ammonia reducer. In some embodiments of any of the methods described herein, the ammonia reducer is an antibiotic (e.g., an antibiotic selected from the group of neomycin, metronidazole, and rifaxamin). In some embodiments of any of the methods described herein, the composition includes about 5 mg to about 2,000 mg of the antibiotic. In some embodiments of any of the methods described herein, the composition includes an ammonia sequestering agent (e.g., an ammonia sequestering agent selected from the group of L-ornithine-L-aspartate, L-ornithine-α-ketoglutarate, and L-ornithine-phenylacetate). In some embodiments of any of the methods described herein, the composition includes about 1 g to about 5 g of the ammonia sequestering agent. In some embodiments of any of the methods described herein, the compositions includes an intestinal deodorizer (e.g., charcoal or copper chlorophyllin). In some embodiments of any of the methods described herein, the composition includes about 10 mg to about 2000 mg of the intestinal deodorizer. In some embodiments of any of the methods described herein, the composition includes a probiotic (e.g., selected from the group of Lactobacillus spp., Bifidobacterium spp., and Streptococcus thermophiles spp.)

In some embodiments of any of the methods described herein, the composition is provided in an oral dosage form (e.g., a tablet or a capsule). In some embodiments of any of the methods described herein, the composition is administered to the subject once, twice, three times, or four times a day.

Also provided herein are capsules that include a therapeutically effective amount of a zeolite and a therapeutically effective amount of one or more additional active ingredients selected from the group of: riboflavin, a colonic acidifier, an ammonia reducer, an ammonia sequestering agent, an intestinal deodorizer, and a probiotic. In some embodiments of any of the capsules described herein, the capsule has a coating that dissolves that at a pH of about 1 to about 3.5.

Also provided herein are kits that include any of the capsules described herein. As used herein, the term “dysregulating mutation” means a mutation in a nucleic acid encoding an enzyme that results in a decrease in the activity of the encoded enzyme as compared to the wildtype enzyme. A mutation in a nucleic acid can result in the production of an encoded enzyme that includes one or more point mutations, the deletion of one or more amino acids, and/or the addition of one or more amino acids. Non-limiting examples of dysregulating mutations in a flavin-containing monooxygenase 3 (FMO3) gene are described herein.

The term “colonic acidifier” means an agent that promotes a decrease in the pH of the colon of a mammal (e.g., a human) following administration (e.g., oral administration) of the agent to the mammal. Non-limiting examples of colonic acidifiers are described herein. Additional examples of colonic acidifiers are known in the art.

The term “ammonia reducer” means an agent that results in a reduction in the production of ammonia in a mammal (e.g., a reduction in the production of ammonia in the gastrointestinal tract of a mammal) following administration (e.g., oral administration) of the agent to the mammal (e.g., as compared to the level of ammonia produced in the gastrointestinal tract of a similar mammal that has not been administered the agent or the level of ammonia produced in the gastrointestinal tract of the mammal prior to administration of the agent). Non-limiting examples of ammonia reducers include antibiotics which reduce the production of ammonia from bacteria present in the gastrointestinal tract of the mammal (e.g., as compared to the level of ammonia produced by bacteria in the gastrointestinal tract of a similar mammal in the absence of the antibiotic or the level of ammonia produced by bacteria in the gastrointestinal tract of the mammal prior to administration of the antibiotic). Non-limiting examples of ammonia reducers are described herein. Additional examples of ammonia reducers are known in the art.

The term “ammonium sequestering agent” means an agent that promotes the removal of ammonia from the gastrointestinal tract of a mammal that has been administered (e.g., orally administered) the agent. For example, an ammonium sequestering agent can be a substrate for an enzyme in a metabolic pathway that also utilizes, in part, ammonium as a substrate. Non-limiting examples of ammonium sequestering agents are described herein. Additional examples of ammonium sequestering agents are known in the art.

The term “intestinal deodorizer” means an agent that promotes the absorption of odor-causing molecules in the intestine of a mammal following administration (e.g., oral administration) of the agent to the mammal. Non-limiting examples of intestinal deodorizers are described herein. Additional examples of intestinal deodorizers are known in the art.

The term “probiotic” means one or more different bacteria that are thought to contribute to provide health benefits to a mammal after administration (e.g., oral administration) to the mammal. Non-limiting examples of probiotics are described herein. Additional examples of probiotics are known in the art.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DETAILED DESCRIPTION

Provided herein are methods of treating TMAU (primary or secondary TMAU), decreasing a level of trimethylamine, increasing a trimethylamine N-oxide ratio, and decreasing a trimethylamine ratio in a subject in need thereof that include orally administering to the subject a therapeutically effective amount of a composition including a zeolite. The composition can be a composition of therapeutic utility that includes one or more (e.g., two, three, four, or five) zeolites (e.g., one or more of any of the zeolites described herein). Also provided herein are compositions (e.g., capsules) that include a therapeutically effective amount of a zeolite, and optionally, a therapeutically effective amount of one or more additional active ingredients selected from the group of: riboflavin, a colonic acidifier, an ammonia reducer, an ammonia sequestering agent, an intestinal deodorizer, and a probiotic.

### Compositions

In some examples, the compositions provided herein can include a zeolite, and optionally, one or more (e.g., one, two, or three) of valerenic acid, carnosic acid, and vitamin E. For example, a composition provided herein can include a zeolite, valerenic acid, carnosic acid, and vitamin E. In some examples, the compositions provided herein can include a zeolite and one or more additional active ingredients containing one or more of riboflavin, a colonic acidifier, an ammonia reducer, an ammonia sequestering agent, an intestinal deodorizer, and a probiotic. In some examples, the compositions provided herein only include one or more zeolites as an active ingredient.

In some examples, any of the compositions described herein can be formulated for oral administration. In some examples, any of the compositions described herein can be formulated in the form of a liquid, semi-liquid, semi-solid, syrup, slurry, chewable form, solution, dispersion, suspension, tablet, pellet, pill, granule, emulsion, gel, a plurality of beads encapsulated in a capsule, powder, suspension, cream, mist, atomized vapor, aerosol, soft gelatin capsule, hard gelatin capsule, a patch, a gel, a confectionary, a shake, a bar, or a supplemented food. In some examples, any of the compositions described herein can be formulated as a tablet or a capsule.

Any of the compositions provided herein can include one or more (e.g., one, two, three, four, or five) zeolites (e.g., one or more of any of the zeolites described herein). The term “zeolite” refers to crystalline aluminosilicate compositions that are microporous and are formed from corner sharing AlO₂ and SiO₂ tetrahedra. Zeolites can include aluminosilicate compounds having various three-dimensional frameworks containing the interlocking SiO and AlO tetrahedrons. The tetrahedrons are cross-linked by the sharing of oxygen atoms such that the ratio of oxygen atoms to the total of the aluminum and silicon atoms is equal to two or O/(Al+Si)=2. The structure of the zeolite can be, e.g., negatively charged and can bind cations and/or positively charged molecules. Zeolites have large voids in their structures that allow entry of large cations, e.g., sodium, potassium, barium, and calcium, and even large positively charged molecules and/or cation groups, e.g., water, ammonia, carbonate ions, and nitrate ions. In some examples, the spaces within the zeolite structure can be interconnected and form long wide channels of varying sizes. These channels can allow easy movement of ions and molecules into and out of the crystalline, highly porous structure of the zeolite. In some examples, the compositions provided herein include one or more zeolites as an active agent. In some examples, the compositions provided herein include one or more zeolites as the only active agent(s) present in the composition.

The general formula of a zeolite is:

M_{e2/n}O.Al₂O₃.xSiO₂.yH₂O,

where:
Mₑ is any alkali or alkaline earth atom;
n is the charge on that atom;
x is the number of Si tetrahedron varying from 1 to 10; and
y is the number of water molecules varying from 1 to 7.

In some cases, a composition provided herein can include zeolites including cation that include any group I or II metal ion, such as Na⁺, K⁺, Li⁺, Ag⁺, NH⁴⁺, H⁺, Ca²⁺, Ba²⁺. In some cases, any one of the compositions provided herein can include a zeolite having an x-value that ranges from about 1.0 to about 10.0. For example, in some examples, the zeolite can include an x-value that ranges from about 1.0 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, or about 1.2, inclusive; about 1.1 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, or about 1.3, inclusive; about 1.2 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, or about 1.4, inclusive; about 1.3 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, or about 1.5, inclusive; about 1.4 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, or about 1.6, inclusive; about 1.5 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, or about 1.7, inclusive; about 1.6 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, or about 1.8, inclusive; about 1.7 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, or about 1.9, inclusive; about 1.8 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, or about 2.0, inclusive; about 1.9 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, or about 2.1, inclusive; about 2.0 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, or about 2.2, inclusive; about 2.1 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, or about 2.3, inclusive; about 2.2 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, or about 2.4, inclusive; about 2.3 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, or about 2.5, inclusive; about 2.5 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, or about 2.7, inclusive; about 2.7 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, or about 2.9, inclusive; about 2.8 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, or about 3.0, inclusive; about 2.9 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, or about 3.1, inclusive; about 3.0 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, or about 3.2, inclusive; about 3.1 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, or about 3.3, inclusive; about 3.2 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, or about 3.4, inclusive; about 3.3 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, or about 3.5, inclusive; about 3.4 about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, or about 3.5, inclusive; about 3.5 about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, or about 3.6, inclusive; about 3.6 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, or about 3.8, inclusive; about 3.7 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, or about 3.9, inclusive; about 3.8 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, or about 4.0, inclusive; about 3.9 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, or about 4.1, inclusive; about 4.0 about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, or about 4.2, inclusive; about 4.1 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, or about 4.3, inclusive; about 4.2 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, or about 4.4, inclusive; about 4.3 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, or about 4.5, inclusive; about 4.4 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, or about 4.6, inclusive; about 4.5 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, or about 4.7, inclusive; about 4.6 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, or about 4.8, inclusive; about 4.7 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, or about 4.9, inclusive; about 4.8 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, or about 5.0, inclusive; about 4.9 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, or about 5.1, inclusive; about 5.0 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, or about 5.2, inclusive; about 5.1 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, or about 5.3, inclusive; about 5.2 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, inclusive; about 5.3 to about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, or about 5.5, inclusive; about 5.4 to about 7.0, about 6.9, about 6.8, about 6.7, about 6.6, about 6.5, about 6.4, about 6.3, about 6.2, about 6.1, about 6.0, about 5.9, about 5.8, about 5.7, or about 5.6, inclusive; about 5.5 to about 7.0, about 6.9, about 6.8, about 6.7, about 6.6, about 6.5, about 6.4, about 6.3, about 6.2, about 6.1, about 6.0, about 5.9, about 5.8, or about 5.7, inclusive; about 5.6 to about 7.0, about 6.9, about 6.8, about 6.7, about 6.6, about 6.5, about 6.4, about 6.3, about 6.2, about 6.1, about 6.0, about 5.9, or about 5.8, inclusive; about 5.7 to about 7.0, about 6.9, about 6.8, about 6.7, about 6.6, about 6.5, about 6.4, about 6.3, about 6.2, about 6.1, about 6.0, or about 5.9, inclusive; about 5.8 to about 7.0, about 6.9, about 6.8, about 6.7, about 6.6, about 6.5, about 6.4, about 6.3, about 6.2, about 6.1, or about 6.0, inclusive; about 5.9 to about 7.0, about 6.9, about 6.8, about 6.7, about 6.6, about 6.5, about 6.4, about 6.3, about 6.2, or about 6.1, inclusive; about 6.0 to about 7.0, about 6.9, about 6.8, about 6.7, about 6.6, about 6.5, about 6.4, about 6.3, or about 6.2, inclusive; about 6.1 to about 7.0, about 6.9, about 6.8, about 6.7, about 6.6, about 6.5, about 6.4, or about 6.3, inclusive; about 6.2 to about 7.0, about 6.9, about 6.8, about 6.7, about 6.6, about 6.5, or about 6.4, inclusive; about 6.3 to about 7.0, about 6.9, about 6.8, about 6.7, about 6.6, or about 6.5, inclusive; about 6.4 to about 7.0, about 6.9, about 6.8, about 6.7, or about 6.6, inclusive; about 6.5 to about 7.0, about 6.9, about 6.8, or about 6.7, inclusive; about 6.6 to about 7.0, about 6.9, or about 6.8, inclusive; about 6.7 to about 7.0, or about 6.9, inclusive; about 6.8 to about 7.0, or about 6.9, inclusive; or about 6.9 to about 7.0, inclusive.

The structural formula of the zeolite based on its crystal unit cell (assuming both the SiO₂ and AlO₂ as variables) can be represented by:

(Mₑ)a/ₙ(AlO₂)ₐ(SiO₂)_{b}.wH₂O,

where:
w is the number of water molecules per unit cell varying from 1 to 10; and
a and b are total numbers of tetrahedra of Al and Si, respectively per unit cell.

In some examples, any of the compositions described herein can include one or more zeolites that include a ratio of b/a that ranges from 1 to 5, 4, 3, or 2, inclusive; from 2 to 5, 4, or 3, inclusive; from 3 to 5 or 4, inclusive; or from 4 to 5, inclusive. In some examples, any of the compositions provided herein can include a zeolite that is a Mordenite, which has a formula of Na₈ (AlO₂)₈(SiO₂)₄₀, where a is 8, b is 40 and thus b/a is 5. In some examples, any of the compositions provided herein can include a Type 4A zeolite, which has a formula of Na₉₆ (AlO₂)₉₆(SiO₂)₉₆, where a is 96, b is 96, and b/a is about 0.9 to about 1.1 (e.g., 1). In some examples, any of the compositions provided herein can include a zeolite having a b/a ratio that ranges from 1 to 100, 90, 80, 70, 60, 50, 40, 30, 20, 10, 9, 8, 7, 6, 5, 4, 3, or 2, inclusive; from 2 to 100, 90, 80, 70, 60, 50, 40, 30, 20, 10, 9, 8, 7, 6, 5, 4, or 3, inclusive; 3 to 100, 90, 80, 70, 60, 50, 40, 30, 20, 10, 9, 8, 7, 6, 5, or 4, inclusive; 4 to 100, 90, 80, 70, 60, 50, 40, 30, 20, 10, 9, 8, 7, 6, or 5, inclusive; 5 to 100, 90, 80, 70, 60, 50, 40, 30, 20, 10, 9, 8, 7, or 6, inclusive; 4 to 100, 90, 80, 70, 60, 50, 40, 30, 20, 10, 9, 8, 7, 6, or 5, inclusive; 5 to 100, 90, 80, 70, 60, 50, 40, 30, 20, 10, 9, 8, 7, or 6, inclusive; 6 to 100, 90, 80, 70, 60, 50, 40, 30, 20, 10, 9, 8, or 7, inclusive; 7 to 100, 90, 80, 70, 60, 50, 40, 30, 20, 10, 9, or 8, inclusive; 8 to 100, 90, 80, 70, 60, 50, 40, 30, 20, 10, or 9, inclusive; 9 to 100, 90, 80, 70, 60, 50, 40, 30, 20, or 10, inclusive; 10 to 100, 90, 80, 70, 60, 50, 40, 30, or 20, inclusive; 20 to 100, 90, 80, 70, 60, 50, 40, or 30, inclusive; 30 to 100, 90, 80, 70, 60, 50, or 40, inclusive; 40 to 100, 90, 80, 70, 60, or 50, inclusive; 50 to 100, 90, 80, 70, or 60, inclusive; 60 to 100, 90, 80, or 70, inclusive; 70 to 100, 90, or 80, inclusive; 80 to 100 or 90, inclusive; or 90 to 100, inclusive. In some examples, any of the compositions described herein can include a zeolite having a b/a ratio that is greater than 100 (for example, a ZSM-5 type zeolite). In some cases, any one of the compositions provided herein can include a zeolite that has a b/a ratio ranging from 0.1 to 5.0, inclusive. For example, in some examples, the zeolite can include a b/a ratio ranging from about 0.1 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, about 1.2, or about 1.1, about 1.0, about 0.9, about 0.8, about 0.7, about 0.6, about 0.5, about 0.4, about 0.3, about 0.2, inclusive; about 0.2 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, inclusive; about 0.3 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, inclusive; about 0.4 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, inclusive; about 0.5 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, inclusive; about 0.6 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, about 1.2, about 1.1, about 1.0, about 0.9, about 0.8, about 0.7, inclusive; about 0.7 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, about 1.2, about 1.1, about 1.0, about 0.9, about 0.8, inclusive; about 0.8 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, about 1.2, about 1.1, about 1.0, about 0.9, inclusive; about 0.9 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, about 1.2, about 1.1, about 1.0, inclusive; about 1.0 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, about 1.2, or about 1.1, inclusive; about 1.1 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, or about 1.2, inclusive; 1.2 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, inclusive; about 1.4 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, or about 1.5, inclusive; about 1.5 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, or about 1.6, inclusive; about 1.6 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, or about 1.7, inclusive; about 1.7 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, or about 1.8, inclusive; about 1.8 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, or about 1.9, inclusive; about 1.9 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, or about 2.0, inclusive; about 2.0 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, or about 2.1, inclusive; about 2.1 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, or about 2.2, inclusive; about 2.2 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, or about 2.3, inclusive; about 2.3 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, or about 2.4, inclusive; about 2.5 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, or about 2.6, inclusive; about 2.6 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, or about 2.7, inclusive; about 2.7 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, or about 2.8, inclusive; about 2.8 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, inclusive; about 2.9 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, or about 3.0, inclusive; about 3.0 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, or about 3.1, inclusive; about 3.1 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, or about 3.2, inclusive; about 3.2 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, or about 3.3, inclusive; about 3.3 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, or about 3.4, inclusive; about 3.4 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, or about 3.5, inclusive; about 3.5 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, or about 3.6, inclusive; about 3.6 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, or about 3.7, inclusive; about 3.7 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, or about 3.8, inclusive; about 3.8 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, or about 3.9, inclusive; about 3.9 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, or about 4.0, inclusive; about 4.0 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, inclusive; about 4.1 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, or about 4.2, inclusive; about 4.2 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, or about 4.3, inclusive; about 4.3 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, or about 4.4, inclusive; about 4.4 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, or about 4.5, inclusive; about 4.5 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, or about 4.6, inclusive; about 4.7 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, or about 4.6, inclusive; about 4.6 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, or about 4.7, inclusive; about 4.7 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, or about 4.8, inclusive; about 4.8 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, or about 4.9, inclusive; about 4.9 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, inclusive; about 5.0 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, or about 5.1, inclusive; about 5.1 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, or about 5.2, inclusive; about 5.2 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, or about 5.3, inclusive; about 5.3 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, or about 5.4, inclusive; about 5.4 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, or about 5.5, inclusive; about 5.5 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, inclusive; about 5.6 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, inclusive; about 5.7 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, or about 5.8, inclusive; about 5.8 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, or about 5.9, inclusive; about 5.9 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, or about 6.0, inclusive; about 6.0 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, or about 6.1, inclusive; about 6.1 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, or about 6.2, inclusive; about 6.3 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, or about 6.4, inclusive; about 6.5 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, inclusive; about 6.6 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, or about 6.7, inclusive; about 6.7 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, or about 6.8, inclusive; about 6.8 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, or about 6.9, inclusive; about 6.9 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, or about 7.0, inclusive; about 7.0 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, or about 7.1, inclusive; about 7.1 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, or about 7.2, inclusive; about 7.3 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, or about 7.4, inclusive; about 7.4 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, or about 7.5, inclusive; about 7.5 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, or about 7.6, inclusive; about 7.6 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, or about 7.5, inclusive; about 7.5 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, or about 7.6, inclusive; about 7.6 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, or about 7.7, inclusive; about 7.7 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, or about 7.8, inclusive; about 7.8 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, or about 7.9, inclusive; about 7.9 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, or about 8.0, inclusive; about 8.0 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, or about 8.1, inclusive; about 8.1 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, or about 8.2, inclusive; about 8.2 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, or about 8.3, inclusive; about 8.3 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, or about 8.4, inclusive; about 8.4 to about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, or about 8.5, inclusive; about 8.5 to about 10.0, about 9.9, about 9.8, about 9.7, about 9.6, about 9.5, about 9.4, about 9.3, about 9.2, about 9.1, about 9.0, about 8.9, about 8.8, about 8.7, or about 8.6, inclusive; about 8.6 to about 10.0, about 9.9, about 9.8, about 9.7, about 9.6, about 9.5, about 9.4, about 9.3, about 9.2, about 9.1, about 9.0, about 8.9, about 8.8, or about 8.7, inclusive; about 8.7 to about 10.0, about 9.9, about 9.8, about 9.7, about 9.6, about 9.5, about 9.4, about 9.3, about 9.2, about 9.1, about 9.0, about 8.9, or about 8.8, inclusive; about 8.8 to about 10.0, about 9.9, about 9.8, about 9.7, about 9.6, about 9.5, about 9.4, about 9.3, about 9.2, about 9.1, about 9.0, or about 8.9, inclusive; about 8.9 to about 10.0, about 9.9, about 9.8, about 9.7, about 9.6, about 9.5, about 9.4, about 9.3, about 9.2, about 9.1, or about 9.0, inclusive; about 9.0 to about 10.0, about 9.9, about 9.8, about 9.7, about 9.6, about 9.5, about 9.4, about 9.3, about 9.2, or about 9.1, inclusive; about 9.1 to about 10.0, about 9.9, about 9.8, about 9.7, about 9.6, about 9.5, about 9.4, about 9.3, or about 9.2, inclusive; about 9.2 to about 10.0, about 9.9, about 9.8, about 9.7, about 9.6, about 9.5, about 9.4, about 9.3, or about 9.2, inclusive; about 9.3 to about 10.0, about 9.9, about 9.8, about 9.7, about 9.6, about 9.5, or about 9.4, inclusive; about 9.4 to about 10.0, about 9.9, about 9.8, about 9.7, about 9.6, or about 9.5, inclusive; about 9.5 to about 10.0, about 9.9, about 9.8, about 9.7, or about 9.6, inclusive; about 9.6 to about 10.0, about 9.9, about 9.8, or about 9.7, inclusive; about 9.7 to about 10.0, about 9.9, about 9.8, about 9.7, or about 9.6, inclusive; about 9.7 to about 10.0, about 9.9, or about 9.8, inclusive; about 9.8 to about 10.0, or about 9.9, inclusive; or about 9.9 to about 10.0, inclusive.

In some examples, any of the compositions provided herein can include a natural zeolite, a synthetic zeolite, or both. Certain types of zeolites are found in nature can also be made synthetically, while other types of zeolites are made only synthetically. In some examples, any of the compositions provided herein can include a zeolite that is synthesized or derived from natural sources. Examples of zeolites that can be included within a composition provided herein include, without limitation, natural zeolites (e.g., chabazite and clinoptilolite), synthetic zeolites, or combinations thereof. Non-limiting methods for synthesizing zeolites are described in, e.g., Tian et al., Z. Anorg. Allg. Chem. 640:1177-1181, 2014; Wu et al., J. Am. Chem. Soc. 136:4019-4025, 2014; Zhang et al., Waste Manag. Res. 31:1160-1169, 2013; Roth et al., Nat. Chem. 5:628-633, 2013; Xu et al., J. Chem. Soc. Chem. Comm. 755-756, 1990; Ma et al., Carbon 40:2367-2374, 2002; U.S. Pat. No. 4,178,352; Zeolites as Catalysts, Sorbents, and Detergent Builders: Applications and Innovations, Karge and Weitkamp, Eds., Eslevier, 1989; Aoki et al., J. Membrane Sci. 141:197-205, 1998.

Any of the compositions provided herein can include, e.g., one or more zeolites selected based on a specific structure type. Zeolites are differentiated mainly according to the geometry of the pores which are formed by the rigid network of the SiO and AlO tetrahedrons. In some examples, the three-dimensional framework of a zeolite included in any of the compositions provided herein can have a particular pore structure that includes, but is not limited to, a Type Y zeolite, a Type F zeolite, a Type J zeolite, a Type M zeolite, a Type Z zeolite, or a Type A zeolite. The Type 3A, Type 4A, and Type 5A zeolites are zeolites having an A structure, while a Type 13X zeolite is a zeolite having an X structure. The A structure is smaller and more square-shaped than the X structure, which is generally larger and circle shaped. The structure of these different zeolites are known in the art. For example, the structures of these different zeolites are described in Fly Ash Zeolites, Springer (2016), Chapter 2.

Any of the compositions provided herein can, e.g., include a zeolite that has been selected based on a desired pore size. In some examples, any of the compositions provided herein can include a Type 3A zeolite, a Type 4A zeolite, Type 5A zeolite, or Type 13X zeolite, or combinations thereof. In some examples, any of the compositions provided herein can include a Type 4A zeolite. A Type 3A zeolite is a zeolite having a pore size of, e.g., about 2.5 angstroms to about 3.4 angstroms, about 3.3 angstroms, about 3.2 angstroms, about 3.1 angstroms, about 3.0 angstroms, about 2.9 angstroms, about 2.8 angstroms, about 2.7 angstroms, or about 2.6 angstroms, inclusive; about 2.6 angstroms to about 3.4 angstroms, about 3.3 angstroms, about 3.2 angstroms, about 3.1 angstroms, about 3.0 angstroms, about 2.9 angstroms, about 2.8 angstroms, or about 2.7 angstroms, inclusive; about 2.7 angstroms to about 3.4 angstroms, about 3.3 angstroms, about 3.2 angstroms, about 3.1 angstroms, about 3.0 angstroms, about 2.9 angstroms, or about 2.8 angstroms, inclusive; about 2.8 angstroms to about 3.4 angstroms, about 3.3 angstroms, about 3.2 angstroms, about 3.1 angstroms, about 3.0 angstroms, or about 2.9 angstroms, inclusive; about 2.9 angstroms to about 3.4 angstroms, about 3.3 angstroms, about 3.2 angstroms, about 3.1 angstroms, or about 3.0 angstroms, inclusive; about 3.0 angstroms to about 3.4 angstroms, about 3.3 angstroms, about 3.2 angstroms, or about 3.1 angstroms, inclusive; about 3.1 angstroms to about 3.4 angstroms, about 3.3 angstroms, or about 3.2 angstroms, inclusive; about 3.2 angstroms to about 3.4 angstroms or about 3.3 angstroms, inclusive; or about 3.3 angstroms to about 3.4 angstroms, inclusive. In some embodiments, a Type 3A zeolite is a zeolite having a pore size of about 3 angstroms.

A Type 4A zeolite is a zeolite having a pore size of, e.g., about 3.5 angstroms to about 4.4 angstroms, about 4.3 angstroms, about 4.2 angstroms, about 4.1 angstroms, about 4.0 angstroms, about 3.9 angstroms, about 3.8 angstroms, about 3.7 angstroms, or about 3.6 angstroms, inclusive; about 3.6 angstroms to about 4.4 angstroms, about 4.3 angstroms, about 4.2 angstroms, about 4.1 angstroms, about 4.0 angstroms, about 3.9 angstroms, about 3.8 angstroms, or about 3.7 angstroms, inclusive; about 3.7 to about 4.4 angstroms, about 4.3 angstroms, about 4.2 angstroms, about 4.1 angstroms, about 4.0 angstroms, about 3.9 angstroms, or about 3.8 angstroms, inclusive; about 3.8 angstroms to about 4.4 angstroms, about 4.3 angstroms, about 4.2 angstroms, about 4.1 angstroms, about 4.0 angstroms, or about 3.9 angstroms, inclusive; about 3.9 angstroms to about 4.4 angstroms, about 4.3 angstroms, about 4.2 angstroms, about 4.1 angstroms, or about 4.0 angstroms, inclusive; about 4.0 angstroms to about 4.4 angstroms, about 4.3 angstroms, about 4.2 angstroms, or about 4.1 angstroms, inclusive; about 4.1 angstroms to about 4.4 angstroms, about 4.3 angstroms, or about 4.2 angstroms, inclusive; about 4.2 angstroms to about 4.4 angstroms or about 4.3 angstroms, inclusive; or about 4.3 angstroms to about 4.4 angstroms, inclusive. In some examples, a Type 4A zeolite has a pore size of about 4 angstroms. An exemplary Type 4A zeolite can include a low silica sodium zeolite (Si/A1 of about 0.9 to about 1.1 (e.g., 1), Na/Al=1, e.g., zeolite 4A) that possesses an open cage within the lattice and a vast network of negatively charged open channels (accommodating Na⁺ cations) due to presence of the common oxygen atom between Si and Al tetrahedra. In addition, because the pores or channels are scaled in microscopic dimensions, these types of zeolites are sometimes referred to as “molecular sieves.”

A Type 5A zeolite is a zeolite having a pore size of, e.g., about 4.5 angstroms to about 5.4 angstroms, about 5.3 angstroms, about 5.2 angstroms, about 5.1 angstroms, about 5.0 angstroms, about 4.9 angstroms, about 4.8 angstroms, about 4.7 angstroms, or about 4.6 angstroms, inclusive; about 4.6 angstroms to about 5.4 angstroms, about 5.3 angstroms, about 5.2 angstroms, about 5.1 angstroms, about 5.0 angstroms, about 4.9 angstroms, about 4.8 angstroms, or about 4.7 angstroms, inclusive; about 4.7 angstroms to about 5.4 angstroms, about 5.3 angstroms, about 5.2 angstroms, about 5.1 angstroms, about 5.0 angstroms, about 4.9 angstroms, or about 4.8 angstroms (inclusive); about 4.8 angstroms to about 5.4 angstroms, about 5.3 angstroms, about 5.2 angstroms, about 5.1 angstroms, about 5.0 angstroms, or about 4.9 angstroms (inclusive); about 4.9 angstroms to about 5.4 angstroms, about 5.3 angstroms, about 5.2 angstroms, about 5.1 angstroms, or about 5.0 angstroms (inclusive); about 5.0 angstroms to about 5.4 angstroms, about 5.3 angstroms, about 5.2 angstroms, or about 5.1 angstroms (inclusive); about 5.1 angstroms to about 5.4 angstroms, about 5.3 angstroms, or about 5.2 angstroms (inclusive); about 5.2 angstroms to about 5.4 angstroms, or about 5.3 angstroms (inclusive); or about 5.3 angstroms to about 5.4 angstroms, inclusive. In some examples, a type 5A zeolite has a pore size of about 5 angstroms.

A Type 13X zeolite is a zeolite having a pore size of, e.g., about 7.5 angstroms to about 8.4 angstroms, about 8.3 angstroms, about 8.2 angstroms, about 8.1 angstroms, about 8.0 angstroms, about 7.9 angstroms, about 7.8 angstroms, about 7.7 angstroms, or about 7.6 angstroms, inclusive; about 7.6 angstroms to about 8.4 angstroms, about 8.3 angstroms, about 8.2 angstroms, about 8.1 angstroms, about 8.0 angstroms, about 7.9 angstroms, about 7.8 angstroms, or about 7.7 angstroms, inclusive; about 7.7 angstroms to about 8.4 angstroms, about 8.3 angstroms, about 8.2 angstroms, about 8.1 angstroms, about 8.0 angstroms, about 7.9 angstroms, or about 7.8 angstroms, inclusive; about 7.8 angstroms to about 8.4 angstroms, about 8.3 angstroms, about 8.2 angstroms, about 8.1 angstroms, about 8.0 angstroms, or about 7.9 angstroms, inclusive; about 7.9 angstroms to about 8.4 angstroms, about 8.3 angstroms, about 8.2 angstroms, about 8.1 angstroms, or about 8.0 angstroms, inclusive; about 8.0 angstroms about 8.4 angstroms, about 8.3 angstroms, about 8.2 angstroms, or about 8.1 angstroms, inclusive; about 8.1 angstroms to about 8.4 angstroms, about 8.3 angstroms, or about 8.2 angstroms, inclusive; about 8.2 angstroms to about 8.4 angstroms or about 8.3 angstroms, inclusive; or about 8.3 angstroms to about 8.4 angstroms, inclusive. In some examples, a Type 13X zeolite has a pore size of about 8 angstroms.

In some examples, the zeolite included in any of the compositions provided herein can have a pore size ranging from about 2.0 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, about 3.9 angstroms, about 3.8 angstroms, about 3.7 angstroms, about 3.6 angstroms, about 3.5 angstroms, about 3.4 angstroms, about 3.3 angstroms, about 3.2 angstroms, about 3.1 angstroms, about 3.0 angstroms, about 2.9 angstroms, about 2.8 angstroms, about 2.7 angstroms, about 2.6 angstroms, about 2.5 angstroms, about 2.4 angstroms, about 2.3 angstroms, about 2.2 angstroms, or about 2.1 angstroms, inclusive; about 2.1 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, about 3.9 angstroms, about 3.8 angstroms, about 3.7 angstroms, about 3.6 angstroms, about 3.5 angstroms, about 3.4 angstroms, about 3.3 angstroms, about 3.2 angstroms, about 3.1 angstroms, about 3.0 angstroms, about 2.9 angstroms, about 2.8 angstroms, about 2.7 angstroms, about 2.6 angstroms, about 2.5 angstroms, about 2.4 angstroms, about 2.3 angstroms, or about 2.2 angstroms, inclusive; about 2.2 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, about 3.9 angstroms, about 3.8 angstroms, about 3.7 angstroms, about 3.6 angstroms, about 3.5 angstroms, about 3.4 angstroms, about 3.3 angstroms, about 3.2 angstroms, about 3.1 angstroms, about 3.0 angstroms, about 2.9 angstroms, about 2.8 angstroms, about 2.7 angstroms, about 2.6 angstroms, about 2.5 angstroms, about 2.4 angstroms, or about 2.3 angstroms, inclusive; about 2.3 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, about 3.9 angstroms, about 3.8 angstroms, about 3.7 angstroms, about 3.6 angstroms, about 3.5 angstroms, about 3.4 angstroms, about 3.3 angstroms, about 3.2 angstroms, about 3.1 angstroms, about 3.0 angstroms, about 2.9 angstroms, about 2.8 angstroms, about 2.7 angstroms, about 2.6 angstroms, about 2.5 angstroms, or about 2.4 angstroms, inclusive; about 2.4 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, about 3.9 angstroms, about 3.8 angstroms, about 3.7 angstroms, about 3.6 angstroms, about 3.5 angstroms, about 3.4 angstroms, about 3.3 angstroms, about 3.2 angstroms, about 3.1 angstroms, about 3.0 angstroms, about 2.9 angstroms, about 2.8 angstroms, about 2.7 angstroms, about 2.6 angstroms, or about 2.5 angstroms, inclusive; about 2.5 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, about 3.9 angstroms, about 3.8 angstroms, about 3.7 angstroms, about 3.6 angstroms, about 3.5 angstroms, about 3.4 angstroms, about 3.3 angstroms, about 3.2 angstroms, about 3.1 angstroms, about 3.0 angstroms, about 2.9 angstroms, about 2.8 angstroms, about 2.7 angstroms, or about 2.6 angstroms, inclusive; about 2.6 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, about 3.9 angstroms, about 3.8 angstroms, about 3.7 angstroms, about 3.6 angstroms, about 3.5 angstroms, about 3.4 angstroms, about 3.3 angstroms, about 3.2 angstroms, about 3.1 angstroms, about 3.0 angstroms, about 2.9 angstroms, about 2.8 angstroms, or about 2.7 angstroms, inclusive; about 2.7 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, about 3.9 angstroms, about 3.8 angstroms, about 3.7 angstroms, about 3.6 angstroms, about 3.5 angstroms, about 3.4 angstroms, about 3.3 angstroms, about 3.2 angstroms, about 3.1 angstroms, about 3.0 angstroms, about 2.9 angstroms, or about 2.8 angstroms, inclusive; about 2.8 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, about 3.9 angstroms, about 3.8 angstroms, about 3.7 angstroms, about 3.6 angstroms, about 3.5 angstroms, about 3.4 angstroms, about 3.3 angstroms, about 3.2 angstroms, about 3.1 angstroms, about 3.0 angstroms, or about 2.9 angstroms, inclusive; about 2.9 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, about 3.9 angstroms, about 3.8 angstroms, about 3.7 angstroms, about 3.6 angstroms, about 3.5 angstroms, about 3.4 angstroms, about 3.3 angstroms, about 3.2 angstroms, about 3.1 angstroms, or about 3.0 angstroms, inclusive; about 3.0 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, about 3.9 angstroms, about 3.8 angstroms, about 3.7 angstroms, about 3.6 angstroms, about 3.5 angstroms, about 3.4 angstroms, about 3.3 angstroms, about 3.2 angstroms, or about 3.1 angstroms, inclusive; about 3.1 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, about 3.9 angstroms, about 3.8 angstroms, about 3.7 angstroms, about 3.6 angstroms, about 3.5 angstroms, about 3.4 angstroms, about 3.3 angstroms, or about 3.2 angstroms, inclusive; about 3.2 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, about 3.9 angstroms, about 3.8 angstroms, about 3.7 angstroms, about 3.6 angstroms, about 3.5 angstroms, about 3.4 angstroms, or about 3.3 angstroms, inclusive; about 3.3 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, about 3.9 angstroms, about 3.8 angstroms, about 3.7 angstroms, about 3.6 angstroms, about 3.5 angstroms, or about 3.4 angstroms, inclusive; about 3.4 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, about 3.9 angstroms, about 3.8 angstroms, about 3.7 angstroms, about 3.6 angstroms, or about 3.5 angstroms, inclusive; about 3.5 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, about 3.9 angstroms, about 3.8 angstroms, about 3.7 angstroms, or about 3.6 angstroms, inclusive; about 3.6 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, about 3.9 angstroms, about 3.8 angstroms, or about 3.7 angstroms, inclusive; about 3.7 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, about 3.9 angstroms, or about 3.8 angstroms, inclusive; about 3.8 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, about 4.0 angstroms, or about 3.9 angstroms, inclusive; about 3.9 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, about 4.1 angstroms, or about 4.0 angstroms, inclusive; about 4.0 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, about 4.2 angstroms, or about 4.1 angstroms, inclusive; about 4.1 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, about 4.3 angstroms, or about 4.2 angstroms, inclusive; about 4.2 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, about 4.4 angstroms, or about 4.3 angstroms, inclusive; about 4.3 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, about 4.5 angstroms, or about 4.4 angstroms, inclusive; about 4.4 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, about 4.6 angstroms, or about 4.5 angstroms, inclusive; about 4.5 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, about 4.7 angstroms, or about 4.6 angstroms, inclusive; about 4.6 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, about 4.8 angstroms, or about 4.7 angstroms, inclusive; about 4.7 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, about 4.9 angstroms, or about 4.8 angstroms, inclusive; about 4.8 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, about 5.0 angstroms, or about 4.9 angstroms, inclusive; about 4.9 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, about 5.1 angstroms, or about 5.0 angstroms, inclusive; about 5.0 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, about 5.2 angstroms, or about 5.1 angstroms, inclusive; about 5.1 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, about 5.3 angstroms, or about 5.2 angstroms, inclusive; about 5.2 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, about 5.4 angstroms, or about 5.3 angstroms, inclusive; about 5.3 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, about 5.5 angstroms, or about 5.4 angstroms, inclusive; about 5.4 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, about 5.6 angstroms, or about 5.5 angstroms, inclusive; about 5.5 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, about 5.7 angstroms, or about 5.6 angstroms, inclusive; about 5.6 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, about 5.8 angstroms, or about 5.7 angstroms, inclusive; about 5.7 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, about 5.9 angstroms, or about 5.8 angstroms, inclusive; about 5.8 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, about 6.0 angstroms, or about 5.9 angstroms, inclusive; about 5.9 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, about 6.1 angstroms, or about 6.0 angstroms, inclusive; about 6.0 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, about 6.2 angstroms, or about 6.1 angstroms, inclusive; about 6.1 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, about 6.3 angstroms, or about 6.2 angstroms, inclusive; about 6.2 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, about 6.4 angstroms, or about 6.3 angstroms, inclusive; about 6.3 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, about 6.5 angstroms, or about 6.4 angstroms, inclusive; about 6.4 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, about 6.6 angstroms, or about 6.5 angstroms, inclusive; about 6.5 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, about 6.7 angstroms, or about 6.6 angstroms, inclusive; about 6.6 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, about 6.8 angstroms, or about 6.7 angstroms, inclusive; about 6.7 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, about 6.9 angstroms, or about 6.8 angstroms, inclusive; about 6.8 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, about 7.0 angstroms, or about 6.9 angstroms, inclusive; about 6.9 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, about 7.1 angstroms, or about 7.0 angstroms, inclusive; about 7.0 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, about 7.2 angstroms, or about 7.1 angstroms, inclusive; about 7.1 angstroms to about 8.0 angstroms, about 7.9 angstroms, about 7.8 angstroms, about 7.7 angstroms, about 7.6 angstroms, about 7.5 angstroms, about 7.4 angstroms, about 7.3 angstroms, or about 7.2 angstroms, inclusive; about 7.2 angstroms to about 8.0 angstroms, about 7.9 angstroms, about 7.8 angstroms, about 7.7 angstroms, about 7.6 angstroms, about 7.5 angstroms, about 7.4 angstroms, or about 7.3 angstroms, inclusive; about 7.3 angstroms to about 8.0 angstroms, about 7.9 angstroms, about 7.8 angstroms, about 7.7 angstroms, about 7.6 angstroms, about 7.5 angstroms, or about 7.4 angstroms, inclusive; about 7.4 angstroms to about 8.0 angstroms, about 7.9 angstroms, about 7.8 angstroms, about 7.7 angstroms, about 7.6 angstroms, or about 7.5 angstroms, inclusive; about 7.5 angstroms to about 8.0 angstroms, about 7.9 angstroms, about 7.8 angstroms, about 7.7 angstroms, or about 7.6 angstroms, inclusive; about 7.6 angstroms to about 8.0 angstroms, about 7.9 angstroms, about 7.8 angstroms, or about 7.7 angstroms, inclusive; about 7.7 angstroms to about 8.0 angstroms, about 7.9 angstroms, or about 7.8 angstroms, inclusive; about 7.8 angstroms to about 8.0 angstroms or about 7.9 angstroms, inclusive; about 7.9 angstroms to about 8.0 angstroms, inclusive.

In some examples, the zeolite contained in any of the compositions provided herein can have a pore size of at least 2.0 angstroms, at least 2.1 angstroms, at least 2.2 angstroms, at least 2.3 angstroms, at least 2.4 angstroms, at least 2.5 angstroms, at least 2.6 angstroms, at least 2.7 angstroms, at least 2.8 angstroms, at least 2.9 angstroms, at least 3.0 angstroms, at least 3.1 angstroms, at least 3.2 angstroms, at least 3.3 angstroms, at least 3.4 angstroms, at least 3.5 angstroms, at least 3.6 angstroms, at least 3.7 angstroms, at least 3.8 angstroms, at least 3.9 angstroms, at least 4.0 angstroms, at least 4.1 angstroms, at least 4.2 angstroms, at least 4.3 angstroms, at least 4.4 angstroms, at least 4.5 angstroms, at least 4.6 angstroms, at least 4.7 angstroms, at least 4.8 angstroms, at least 4.9 angstroms, at least 5.0 angstroms, at least 5.1 angstroms, at least 5.2 angstroms, at least 5.3 angstroms, at least 5.4 angstroms, at least 5.5 angstroms, at least 5.6 angstroms, at least 5.7 angstroms, at least 5.8 angstroms, at least 5.9 angstroms, at least 6.0 angstroms, at least 6.1 angstroms, at least 6.2 angstroms, at least 6.3 angstroms, at least 6.4 angstroms, at least 6.5 angstroms, at least 6.6 angstroms, at least 6.7 angstroms, at least 6.8 angstroms, at least 6.9 angstroms, at least 7.0 angstroms, at least 7.1 angstroms, at least 7.2 angstroms, at least 7.3 angstroms, at least 7.4 angstroms, at least 7.5 angstroms, at least 7.6 angstroms, at least 7.7 angstroms, at least 7.8 angstroms, at least 7.9 angstroms, or at least 8.0 angstroms.

In some examples, the zeolite contained in any of the compositions provided herein can have a pore size of no more than 8.0 angstroms, no more than 7.9 angstroms, no more than 7.8 angstroms, no more than 7.7 angstroms, no more than 7.6 angstroms, no more than 7.5 angstroms, not more than 7.4 angstroms, no more than 7.3 angstroms, no more than 7.2 angstroms, no more than 7.1 angstroms, no more than 7.0 angstroms, no more than 6.9 angstroms, no more than 6.8 angstroms, no more than 6.7 angstroms, no more than 6.6 angstroms, no more than 6.5 angstroms, no more than 6.4 angstroms, no more than 6.3 angstroms, no more than 6.2 angstroms, no more than 6.1 angstroms, no more than 6.0 angstroms, no more than 5.9 angstroms, no more than 5.8 angstroms, no more than 5.7 angstroms, no more than 5.6 angstroms, no more than 5.5 angstroms, no more than 5.4 angstroms, no more than 5.3 angstroms, no more than 5.2 angstroms, no more than 5.1 angstroms, no more than 5.0 angstroms, no more than 4.9 angstroms, no more than 4.8 angstroms, no more than 4.7 angstroms, no more than 4.6 angstroms, no more than 4.5 angstroms, no more than 4.4 angstroms, no more than 4.3 angstroms, no more than 4.2 angstroms, no more than 4.1 angstroms, no more than 4.0 angstroms, no more than 3.9 angstroms, no more than 3.8 angstroms, no more than 3.7 angstroms, no more than 3.6 angstroms, no more than 3.5 angstroms, no more than 3.4 angstroms, no more than 3.3 angstroms, no more than 3.2 angstroms, no more than 3.1 angstroms, or no more than 3.0 angstroms. Methods for determining the structure and pore size of a zeolite are known in the art. For example, x-ray crystallography can be used to determine the structure and pore size of a zeolite. Other methods for determining the structure and pore size of a zeolite are known in the art.

Any of the compositions provided herein can include one or more zeolites that are, e.g., negatively charged. In some examples, any of the compositions provided herein can include one or more zeolites that are, e.g., positively charged. The charge of the zeolite can, e.g., depend on the pH. For example, in some examples, the zeolite can be negatively charged at a pH of about 1.0 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, or about 1.2, inclusive; about 1.1 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, or about 1.3, inclusive; about 1.2 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, or about 1.4, inclusive; about 1.3 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, or about 1.5, inclusive; about 1.4 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, or about 1.6, inclusive; about 1.5 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, or about 1.7, inclusive; about 1.6 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, or about 1.8, inclusive; about 1.7 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, or about 1.9, inclusive; about 1.8 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, or about 2.0, inclusive; about 1.9 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, or about 2.1, inclusive; about 2.0 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, or about 2.2, inclusive; about 2.1 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, or about 2.3, inclusive; about 2.2 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, or about 2.4, inclusive; about 2.3 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, or about 2.5, inclusive; about 2.5 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, or about 2.7, inclusive; about 2.7 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, or about 2.9, inclusive; about 2.8 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, or about 3.0, inclusive; about 2.9 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, or about 3.1, inclusive; about 3.0 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, or about 3.2, inclusive; about 3.1 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, or about 3.3, inclusive; about 3.2 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, or about 3.4, inclusive; about 3.3 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, or about 3.5, inclusive; about 3.4 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, or about 3.6, inclusive; about 3.6 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, or about 3.8, inclusive; about 3.7 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, or about 3.9, inclusive; about 3.8 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, or about 4.0, inclusive; about 3.9 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, or about 4.1, inclusive; about 4.0 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, or about 4.2, inclusive; about 4.1 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, or about 4.3, inclusive; about 4.2 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, or about 4.4, inclusive; about 4.3 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, or about 4.5, inclusive; about 4.4 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, or about 4.6, inclusive; about 4.5 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, or about 4.7, inclusive; about 4.6 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, or about 4.8, inclusive; about 4.7 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, or about 4.9, inclusive; about 4.8 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, or about 5.0, inclusive; about 4.9 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, or about 5.1, inclusive; about 5.0 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, or about 5.2, inclusive; about 5.1 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, or about 5.3, inclusive; about 5.2 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, inclusive; about 5.3 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, or about 5.5, inclusive; about 5.4 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, or about 5.6, inclusive; about 5.5 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, or about 5.7, inclusive; about 5.6 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, or about 5.8, inclusive; about 5.7 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, or about 5.9, inclusive; about 5.8 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, or about 6.0, inclusive; about 5.9 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, or about 6.1, inclusive; about 6.0 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, or about 6.2, inclusive; about 6.1 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, or about 6.3, inclusive; about 6.2 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, or about 6.4, inclusive; about 6.3 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, or about 6.5, inclusive; about 6.4 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, or about 6.6, inclusive; about 6.5 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, or about 6.7, inclusive; about 6.6 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, or about 6.8, inclusive; about 6.7 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, or about 6.9, inclusive; about 6.8 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, or about 7.0, inclusive; about 6.9 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, or about 7.1, inclusive; about 7.0 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, or about 7.2, inclusive; about 7.1 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, or about 7.3, inclusive; about 7.2 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, or about 7.4, inclusive; about 7.3 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, inclusive; about 7.4 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, or about 7.6, inclusive; about 7.5 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, or about 7.7, inclusive; about 7.8 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, or about 8.0, inclusive; about 7.9 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, or about 8.1, inclusive; about 8.1 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, or about 8.3, inclusive; about 8.2 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, or about 8.4, inclusive; about 8.3 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, or about 8.5, inclusive; about 8.4 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, or about 8.6, inclusive; about 8.5 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, or about 8.7, inclusive; about 8.6 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, or about 8.8, inclusive; about 8.7 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, or about 8.9, inclusive; about 8.8 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, or about 9.0, inclusive; about 8.9 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, or about 9.1, inclusive; about 9.0 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, or about 9.2, inclusive; about 9.1 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, or about 9.3, inclusive; about 9.2 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, or about 9.4, inclusive; about 9.3 to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, or about 9.5, inclusive; about 9.4 to about 11.0, about 10.9, about 10.8, about 10.7, about 10.6, about 10.5, about 10.4, about 10.3, about 10.2, about 10.1, about 10.0, about 9.9, about 9.8, about 9.7, or about 9.6, inclusive; about 9.5 to about 11.0, about 10.9, about 10.8, about 10.7, about 10.6, about 10.5, about 10.4, about 10.3, about 10.2, about 10.1, about 10.0, about 9.9, about 9.8, or about 9.7, inclusive; about 9.6 to about 11.0, about 10.9, about 10.8, about 10.7, about 10.6, about 10.5, about 10.4, about 10.3, about 10.2, about 10.1, about 10.0, about 9.9, or about 9.8, inclusive; about 9.7 to about 11.0, about 10.9, about 10.8, about 10.7, about 10.6, about 10.5, about 10.4, about 10.3, about 10.2, about 10.1, about 10.0, or about 9.9, inclusive; about 9.8 to about 11.0, about 10.9, about 10.8, about 10.7, about 10.6, about 10.5, about 10.4, about 10.3, about 10.2, about 10.1, or about 10.0, inclusive; about 9.9 to about 11.0, about 10.9, about 10.8, about 10.7, about 10.6, about 10.5, about 10.4, about 10.3, about 10.2, or about 10.1, inclusive; about 10.0 to about 11.0, about 10.9, about 10.8, about 10.7, about 10.6, about 10.5, about 10.4, about 10.3, or about 10.2, inclusive; about 10.1 to about 11.0, about 10.9, about 10.8, about 10.7, about 10.6, about 10.5, about 10.4, or about 10.3, inclusive; about 10.2 to about 11.0, about 10.9, about 10.8, about 10.7, about 10.6, about 10.5, or about 10.4, inclusive; about 10.3 to about 11.0, about 10.9, about 10.8, about 10.7, about 10.6, or about 10.5, inclusive; about 10.4 to about 11.0, about 10.9, about 10.8, about 10.7, or about 10.6, inclusive; about 10.5 to about 11.0, about 10.9, about 10.8, or about 10.7, inclusive; about 10.6 to about 11.0, about 10.9, or about 10.8, inclusive; about 10.7 to about 11.0 or about 10.9, inclusive; or about 10.8 to about 11.0, inclusive. Methods for determining the charge of a zeolite (e.g., a negatively charged zeolite) are known in the art.

In some examples, any of the compositions provided herein can optionally include a zeolite suspension or a zeolite slurry including one or more solutions. Suitable solutions can include, e.g., an acidic solution, a neutral solution, a basic solution, an inorganic acid, an organic acid, an acid anhydride, or an acidic salt, or combinations thereof. Exemplary solutions can include, but are not limited to, a sodium aluminate solution, a sodium silicate solution, a sodium water-glass solution, a sodium aluminate solution, sulfuric acid, sulfurous acid, hydrochloric acid, hydrobromic acid, hypochlorous acid, chloric acid, boric acid, nitric acid, phosphoric acid, phosphorous acid, metaphosphoric acid, acetic acid, propionic acid, oxalic acid, citric acid, tartaric acid, succinic acid, maleic acid, malic acid, gluconic acid, itaconic acid, thioglycolic acid, ethylenediamine-tetraacetic acid, nitrilotriacetic acid, diglycolic acid, sulfoitaconic acid, trimellitic acid, pyromellitic acid, acetic anhydride, succinic anhydride, water, or a sodium hydroxide solution, or combinations thereof. In some embodiments of any of the compositions provided herein, about 0.1% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, about 10.0% w/w, about 9.0% w/w, about 8.0% w/w, about 7.0% w/w, about 6.0% w/w, about 5.0% w/w, about 4.0% w/w, about 3.0% w/w, about 2.0% w/w w/w, about 1.0% w/w, about 0.9% w/w, about 0.8% w/w, about 0.7% w/w, about 0.6% w/w, about 0.5% w/w, about 0.4% w/w, about 0.3% w/w, or about 0.2% w/w, inclusive; about 0.2% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, about 10.0% w/w, about 9.0% w/w, about 8.0% w/w, about 7.0% w/w, about 6.0% w/w, about 5.0% w/w, about 4.0% w/w, about 3.0% w/w, about 2.0% w/w, about 1.0% w/w, about 0.9% w/w, about 0.8% w/w, about 0.7% w/w, about 0.6% w/w, about 0.5% w/w, about 0.4% w/w, or about 0.3% w/w, inclusive; about 0.3% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, about 10.0% w/w, about 9.0% w/w, about 8.0% w/w, about 7.0% w/w, about 6.0% w/w, about 5.0% w/w, about 4.0% w/w, about 3.0% w/w, about 2.0% w/w, about 1.0% w/w, about 0.9% w/w, about 0.8% w/w, about 0.7% w/w, about 0.6% w/w, about 0.5% w/w, or about 0.4% w/w, inclusive; about 0.4% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, about 10.0% w/w, about 9.0% w/w, about 8.0% w/w, about 7.0% w/w, about 6.0% w/w, about 5.0% w/w, about 4.0% w/w, about 3.0% w/w, about 2.0% w/w, about 1.0% w/w, about 0.9% w/w, about 0.8% w/w, about 0.7% w/w, about 0.6% w/w, or about 0.5% w/w, inclusive; about 0.5% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, about 10.0% w/w, about 9.0% w/w, about 8.0% w/w, about 7.0% w/w, about 6.0% w/w, about 5.0% w/w, about 4.0% w/w, about 3.0% w/w, about 2.0% w/w w, about 1.0% w/w, about 0.9% w/w, about 0.8% w/w, about 0.7% w/w, or about 0.6% w/w, inclusive; about 0.5% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, about 10.0% w/w, about 9.0% w/w, about 8.0% w/w, about 7.0% w/w, about 6.0% w/w, about 5.0% w/w, about 4.0% w/w, about 3.0% w/w, about 2.0% w/w, about 1.0% w/w, about 0.9% w/w, about 0.8% w/w, about 0.7% w/w, or about 0.6% w/w, inclusive; about 0.6% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, about 10.0% w/w, about 9.0% w/w, about 8.0% w/w, about 7.0% w/w, about 6.0% w/w, about 5.0% w/w, about 4.0% w/w, about 3.0% w/w, about 2.0% w/w w/w, about 1.0% w/w, about 0.9% w/w, about 0.8% w/w, or about 0.7% w/w, inclusive; about 0.7% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, about 10.0% w/w, about 9.0% w/w, about 8.0% w/w, about 7.0% w/w, about 6.0% w/w, about 5.0% w/w, about 4.0% w/w, about 3.0% w/w, about 2.0% w/w 25% w/w, 22% w/w, about 20% w/w, about 18% w/w, about 17% w/w, about 16% w/w, about 15% w/w, about 14% w/w, about 13% w/w, about 12% w/w, about 11% w/w, about 10% w/w, about 9% w/w, about 8% w/w, about 7% w/w, about 6% w/w, about 5% w/w, about 4% w/w, about 3% w/w, about 2% w/w, about 1.0% w/w, about 0.9% w/w, or about 0.8% w/w, inclusive; about 0.8% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, about 10.0% w/w, about 9.0% w/w, about 8.0% w/w, about 7.0% w/w, about 6.0% w/w, about 5.0% w/w, about 4.0% w/w, about 3.0% w/w, about 2.0% w/w, about 1.0% w/w, or about 0.9% w/w, inclusive; about 0.9% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, about 10.0% w/w, about 9.0% w/w, about 8.0% w/w, about 7.0% w/w, about 6.0% w/w, about 5.0% w/w, about 4.0% w/w, about 3.0% w/w, about 2.0% w/w, or about 1.0% w/w, inclusive; about 1.0% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, about 10.0% w/w, about 9.0% w/w, about 8.0% w/w, about 7.0% w/w, about 6.0% w/w, about 5.0% w/w, about 4.0% w/w, about 3.0% w/w, or about 2.0% w/w, inclusive; about 2.0% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, about 10.0% w/w, about 9.0% w/w, about 8.0% w/w, about 7.0% w/w, about 6.0% w/w, about 5.0% w/w, about 4.0% w/w, or about 3.0% w/w, inclusive; about 3.0% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, about 10.0% w/w, about 9.0% w/w, about 8.0% w/w, about 7.0% w/w, about 6.0% w/w, about 5.0% w/w, or about 4.0% w/w, inclusive; about 4.0% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, about 10.0% w/w, about 9.0% w/w, about 8.0% w/w, about 7.0% w/w, about 6.0% w/w, or about 5.0% w/w, inclusive; about 5.0% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, about 10.0% w/w, about 9.0% w/w, about 8.0% w/w, about 7.0% w/w, or about 6.0% w/w, inclusive; about 6.0% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, about 10.0% w/w, about 9.0% w/w, about 8.0% w/w, or about 7.0% w/w, inclusive; about 7.0% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, about 10.0% w/w, about 9.0% w/w, or about 8.0% w/w, inclusive; about 8.0% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, about 10.0% w/w, or about 9.0% w/w, inclusive; about 9.0% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, about 11.0% w/w, or about 10.0% w/w, inclusive; about 10.0% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, about 12.0% w/w, or about 11.0% w/w, inclusive; about 11.0% w/w to about 25.0% w/w, about 22.0% w/w, about 20.0% w/w, about 18.0% w/w, about 17.0% w/w, about 16.0% w/w, about 15.0% w/w, about 14.0% w/w, about 13.0% w/w, or about 12.0% w/w, inclusive; about 12.0% w/w to about 25.0% w/w, about 22.0% w/w, about 20.0% w/w, about 18.0% w/w, about 17.0% w/w, about 16.0% w/w, about 15.0% w/w, about 14.0% w/w, or about 13.0% w/w, inclusive; about 13.0% w/w to about 25.0% w/w, about 22.0% w/w, about 20.0% w/w, about 18.0% w/w, about 17.0% w/w, about 16.0% w/w, about 15.0% w/w, or about 14.0% w/w, inclusive; about 14.0% w/w to about 25.0% w/w, about 22.0% w/w, about 20.0% w/w, about 18.0% w/w, about 17.0% w/w, about 16.0% w/w, or about 15.0% w/w, inclusive; about 15.0% w/w to about 25.0% w/w, about 22.0% w/w, about 20.0% w/w, about 18.0% w/w, about 17.0% w/w, or about 16.0% w/w, inclusive; about 16.0% w/w to about 25.0% w/w, about 22.0% w/w, about 20.0% w/w, about 18.0% w/w, or about 17.0% w/w, inclusive; about 17.0% w/w to about 25.0% w/w, about 22.0% w/w, about 20.0% w/w, or about 18.0% w/w, inclusive; about 18.0% w/w to about 25.0% w/w, about 22.0% w/w, or about 20.0% w/w, inclusive; about 20.0% w/w to about 25.0% w/w or about 22.0% w/w, inclusive; or about 22.0% w/w to about 25.0% w/w, inclusive; of the composition (e.g., a zeolite suspension or a zeolite slurry) is represented by a zeolite (e.g., any of the zeolites described herein).

The one or more solutions, or a zeolite suspension or a zeolite slurry including the one or more solutions provided herein, can have any of the pH values described herein for negatively charging a zeolite. In some cases, the pH value of a zeolite suspension or zeolite slurry at the time of completion of addition of the one or more solutions provided herein is in the range of from about 9.5 to about 12, e.g., about 11.3 to about 12.7. In some examples, the one or more solutions or mixtures thereof, or a zeolite suspension or a zeolite slurry including the one or more solutions provided herein or mixtures thereof, can have a pH of about 9.0 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, or about 9.1, inclusive; about 9.1 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, or about 9.2, inclusive; about 9.2 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, or about 9.3, inclusive; about 9.3 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, or about 9.4, inclusive; about 9.4 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, or about 9.5, inclusive; about 9.5 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, about 10.9, about 10.8, about 10.7, about 10.6, about 10.5, about 10.4, about 10.3, about 10.2, about 10.1, about 10.0, about 9.9, about 9.8, about 9.7, or about 9.6, inclusive; about 9.6 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, about 10.9, about 10.8, about 10.7, about 10.6, about 10.5, about 10.4, about 10.3, about 10.2, about 10.1, about 10.0, about 9.9, about 9.8, or about 9.7, inclusive; about 9.7 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, about 10.9, about 10.8, about 10.7, about 10.6, about 10.5, about 10.4, about 10.3, about 10.2, about 10.1, about 10.0, about 9.9, or about 9.8, inclusive; about 9.8 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, about 10.9, about 10.8, about 10.7, about 10.6, about 10.5, about 10.4, about 10.3, about 10.2, about 10.1, about 10.0, or about 9.9, inclusive; about 9.9 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, about 10.9, about 10.8, about 10.7, about 10.6, about 10.5, about 10.4, about 10.3, about 10.2, about 10.1, or about 10.0, inclusive; about 10.0 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, about 10.9, about 10.8, about 10.7, about 10.6, about 10.5, about 10.4, about 10.3, about 10.2, or about 10.1, inclusive; about 10.1 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, about 10.9, about 10.8, about 10.7, about 10.6, about 10.5, about 10.4, about 10.3, or about 10.2, inclusive; about 10.2 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, about 10.9, about 10.8, about 10.7, about 10.6, about 10.5, about 10.4, or about 10.3, inclusive; about 10.3 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, about 10.9, about 10.8, about 10.7, about 10.6, about 10.5, or about 10.4, inclusive; about 10.4 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, about 10.9, about 10.8, about 10.7, about 10.6, or about 10.5, inclusive; about 10.5 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, about 10.9, about 10.8, about 10.7, or about 10.6, inclusive; about 10.6 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, about 10.9, about 10.8, or about 10.7, inclusive; about 10.7 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, about 10.9, or about 10.8, inclusive; about 10.8 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, about 11.0, or about 10.9, inclusive; about 10.9 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, about 11.1, or about 11.0, inclusive; about 11.0 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, about 11.2, or about 11.1, inclusive; about 11.1 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, about 11.3, or about 11.2, inclusive; about 11.2 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, about 11.4, or about 11.3, inclusive; about 11.3 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, about 11.5, or about 11.4, inclusive; about 11.4 to about 13.0, about 12.9, about 12.8, about 12.7, about 12.6, about 12.5, about 12.4, about 12.3, about 12.2, about 12.1, about 12.0, about 11.9, about 11.8, about 11.7, about 11.6, or about 11.5, inclusive; about 11.5 to about 13.0, about 12.9, about 12.8, about 12.7, about 12.6, about 12.5, about 12.4, about 12.3, about 12.2, about 12.1, about 12.0, about 11.9, about 11.8, about 11.7, or about 11.6, inclusive; about 11.6 to about 13.0, about 12.9, about 12.8, about 12.7, about 12.6, about 12.5, about 12.4, about 12.3, about 12.2, about 12.1, about 12.0, about 11.9, about 11.8, or about 11.7, inclusive; about 11.7 to about 13.0, about 12.9, about 12.8, about 12.7, about 12.6, about 12.5, about 12.4, about 12.3, about 12.2, about 12.1, about 12.0, about 11.9, or about 11.8, inclusive; about 11.8 to about 13.0, about 12.9, about 12.8, about 12.7, about 12.6, about 12.5, about 12.4, about 12.3, about 12.2, about 12.1, about 12.0, or about 11.9, inclusive; about 11.9 to about 13.0, about 12.9, about 12.8, about 12.7, about 12.6, about 12.5, about 12.4, about 12.3, about 12.2, about 12.1, or about 12.0, inclusive; about 12.0 to about 13.0, about 12.9, about 12.8, about 12.7, about 12.6, about 12.5, about 12.4, about 12.3, about 12.2, or about 12.1, inclusive; about 12.1 to about 13.0, about 12.9, about 12.8, about 12.7, about 12.6, about 12.5, about 12.4, about 12.3, or about 12.2, inclusive; about 12.2 to about 13.0, about 12.9, about 12.8, about 12.7, about 12.6, about 12.5, about 12.4, or about 12.3, inclusive; about 12.3 to about 13.0, about 12.9, about 12.8, about 12.7, about 12.6, about 12.5, or about 12.4, inclusive; about 12.4 to about 13.0, about 12.9, about 12.8, about 12.7, about 12.6, or about 12.5, inclusive; about 12.5 to about 13.0, about 12.9, about 12.8, about 12.7, or about 12.6, inclusive; about 12.6 to about 13.0, about 12.9, about 12.8, or about 12.7, inclusive; about 12.7 to about 13.0, about 12.9, or about 12.8, inclusive; about 12.8 to about 13.0 or about 12.9, inclusive; or about 12.9 to about 13.0, inclusive.

The one or more solutions provided herein, or a zeolite suspension or a zeolite slurry including the solutions provided herein, can optionally include a water-soluble or water-dispersible organic polymeric dispersant in an amount of about 0.1% w/w to about 2.0% w/w, about 1.9% w/w, about 1.8% w/w, about 1.7% w/w, about 1.6% w/w, about 1.5% w/w, about 1.4% w/w, about 1.3% w/w, about 1.2% w/w, about 1.1% w/w, about 1.0% w/w, about 0.9% w/w, about 0.8% w/w, about 0.7% w/w, about 0.6% w/w, about 0.5% w/w, about 0.4% w/w, about 0.3% w/w, or about 0.2% w/w, inclusive; about 0.2% w/w to about 2.0% w/w, about 1.9% w/w, about 1.8% w/w, about 1.7% w/w, about 1.6% w/w, about 1.5% w/w, about 1.4% w/w, about 1.3% w/w, about 1.2% w/w, about 1.1% w/w, about 1.0% w/w, about 0.9% w/w, about 0.8% w/w, about 0.7% w/w, about 0.6% w/w, about 0.5% w/w, about 0.4% w/w, or about 0.3% w/w, inclusive; about 0.3% w/w to about 2.0% w/w, about 1.9% w/w, about 1.8% w/w, about 1.7% w/w, about 1.6% w/w, about 1.5% w/w, about 1.4% w/w, about 1.3% w/w, about 1.2% w/w, about 1.1% w/w, about 1.0% w/w, about 0.9% w/w, about 0.8% w/w, about 0.7% w/w, about 0.6% w/w, about 0.5% w/w, or about 0.4% w/w, inclusive; about 0.5% w/w to about 2.0% w/w, about 1.9% w/w, about 1.8% w/w, about 1.7% w/w, about 1.6% w/w, about 1.5% w/w, about 1.4% w/w, about 1.3% w/w, about 1.2% w/w, about 1.1% w/w, about 1.0% w/w, about 0.9% w/w, about 0.8% w/w, about 0.7% w/w, or about 0.6% w/w, inclusive; about 0.6% w/w to about 2.0% w/w, about 1.9% w/w, about 1.8% w/w, about 1.7% w/w, about 1.6% w/w, about 1.5% w/w, about 1.4% w/w, about 1.3% w/w, about 1.2% w/w, about 1.1% w/w, about 1.0% w/w, about 0.9% w/w, about 0.8% w/w, or about 0.7% w/w, inclusive; about 0.7% w/w to about 2.0% w/w, about 1.9% w/w, about 1.8% w/w, about 1.7% w/w, about 1.6% w/w, about 1.5% w/w, about 1.4% w/w, about 1.3% w/w, about 1.2% w/w, about 1.1% w/w, about 1.0% w/w, about 0.9% w/w, or about 0.8% w/w, inclusive; about 0.8% w/w to about 2.0% w/w, about 1.9% w/w, about 1.8% w/w, about 1.7% w/w, about 1.6% w/w, about 1.5% w/w, about 1.4% w/w, about 1.3% w/w, about 1.2% w/w, about 1.1% w/w, about 1.0% w/w, or about 0.9% w/w, inclusive; about 0.8% w/w to about 2.0% w/w, about 1.9% w/w, about 1.8% w/w, about 1.7% w/w, about 1.6% w/w, about 1.5% w/w, about 1.4% w/w, about 1.3% w/w, about 1.2% w/w, about 1.1% w/w, about 1.0% w/w, or about 0.9% w/w, inclusive; about 0.9% w/w to about 2.0% w/w, about 1.9% w/w, about 1.8% w/w, about 1.7% w/w, about 1.6% w/w, about 1.5% w/w, about 1.4% w/w, about 1.3% w/w, about 1.2% w/w, about 1.1% w/w, or about 1.0% w/w, inclusive; about 1.0% w/w to about 2.0% w/w, about 1.9% w/w, about 1.8% w/w, about 1.7% w/w, about 1.6% w/w, about 1.5% w/w, about 1.4% w/w, about 1.3% w/w, about 1.2% w/w, or about 1.1% w/w, inclusive; about 1.1% w/w to about 2.0% w/w, about 1.9% w/w, about 1.8% w/w, about 1.7% w/w, about 1.6% w/w, about 1.5% w/w, about 1.4% w/w, about 1.3% w/w, or about 1.2% w/w, inclusive; about 1.2% w/w to about 2.0% w/w, about 1.9% w/w, about 1.8% w/w, about 1.7% w/w, about 1.6% w/w, about 1.5% w/w, about 1.4% w/w, or about 1.3% w/w, inclusive; about 1.3% w/w to about 2.0% w/w, about 1.9% w/w, about 1.8% w/w, about 1.7% w/w, about 1.6% w/w, about 1.5% w/w, or about 1.4% w/w, inclusive; about 1.4% w/w to about 2.0% w/w, about 1.9% w/w, about 1.8% w/w, about 1.7% w/w, about 1.6% w/w, or about 1.5% w/w, inclusive; about 1.5% w/w to about 2.0% w/w, about 1.9% w/w, about 1.8% w/w, about 1.7% w/w, or about 1.6% w/w, inclusive; about 1.6% w/w to about 2.0% w/w, about 1.9% w/w, about 1.8% w/w, or about 1.7% w/w, inclusive; about 1.7% w/w to about 2.0% w/w, about 1.9% w/w, or about 1.8% w/w, inclusive; about 1.8% w/w to about 2.0% w/w or about 1.9% w/w, inclusive; or about 1.9% w/w to about 2.0% w/w, inclusive.

Suitable water-soluble or water-dispersible organic polymeric dispersant can be, e.g., water-soluble polymers having at least one of carboxyl, hydroxyl and ether groups, such as, e.g., carboxymethyl cellulose (CMC), methyl cellulose, ethyl cellulose, hydroxyethyl cellulose, various starches, cyanoethylated starch, carboxymethylated starch, sodium alginate, tragacanth gum, gum arabic, glue, casein, gelatin, polyvinyl alcohol, partially hydrolyzed polyvinyl acetate, partially acetalized polyvinyl alcohol, polyvinylmethyl ether, a vinylmethyl ether/maleic acid copolymer, polyvinyl pyrrolidone, polyacrylamide, or a water-soluble acrylic resin, or a combination thereof. In some cases, the zeolite slurry or suspension provided herein and include organic dispersants containing polymers having both the carboxyl and hydroxyl groups, such as, e.g., carboxymethyl cellulose or carboxymethylated starch. A composition provided herein can include a zeolite as an active ingredient. In some examples, a composition can include multiple active ingredients in which a zeolite is one of the active ingredients. For example, a composition provided herein can further include one or more additional active ingredients. For example in some embodiments, a composition can further include (in addition to a zeolite) one or more additional active agents selected from the group of valerenic acid, carnosic acid, and vitamin E.

In some examples, the zeolite is the only active ingredient present in the composition provided herein. Without being bound by any particular theory, a zeolite may be useful in treating TMAU (e.g., primary trimethylaminuria and/or secondary trimethylaminuria) in a subject. In some examples, a zeolite may be useful in decreasing a level of trimethylamine, decreasing a level of trimethylamine precursor, increasing a trimethylamine N-oxide ratio, and/or decreasing a trimethylamine ratio in the subject.

Zeolites can be generated using methods known in the art (e.g., as described in U.S. Pat. No. 3,094,383). In some examples, a zeolite can be purchased from a commercial vendor. For example, zeolites can be obtained from Zeolite Products (Arnhem, Netherlands).

In some examples, a composition provided herein can contain an effective amount (e.g., a therapeutically effective amount) of one or more zeolite. A “therapeutically effective amount” as used herein includes a dosage sufficient to result in one or more of: decreasing a level of trimethylamine, decreasing a level of a trimethylamine precursor such as trimethylamine N-oxide (TMAO) or choline, increasing a trimethylamine N-oxide ratio, and decreasing a trimethylamine ratio in a subject (e.g., as compared to the same parameter in the subject prior to administration of any of the compositions described herein or in a subject or a population of subjects having the same TMAU syndrome but receiving a different treatment or a placebo).

In some embodiments of any of the compositions provided herein, about 0.5% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 9.5% w/w, about 9.0% w/w, about 8.5% w/w, about 8.0% w/w, about 7.5% w/w, about 7.0% w/w, about 6.5% w/w, about 6.0% w/w, about 5.5% w/w, about 5.0% w/w, about 4.5% w/w, about 4.0% w/w, about 3.5% w/w, about 3.0% w/w, about 2.5% w/w, about 2.0% w/w, about 1.5% w/w, or about 1.0% w/w, inclusive; about 1.0% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 9.5% w/w, about 9.0% w/w, about 8.5% w/w, about 8.0% w/w, about 7.5% w/w, about 7.0% w/w, about 6.5% w/w, about 6.0% w/w, about 5.5% w/w, about 5.0% w/w, about 4.5% w/w, about 4.0% w/w, about 3.5% w/w, about 3.0% w/w, about 2.5% w/w, about 2.0% w/w, or about 1.5% w/w, inclusive; about 1.5% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 9.5% w/w, about 9.0% w/w, about 8.5% w/w, about 8.0% w/w, about 7.5% w/w, about 7.0% w/w, about 6.5% w/w, about 6.0% w/w, about 5.5% w/w, about 5.0% w/w, about 4.5% w/w, about 4.0% w/w, about 3.5% w/w, about 3.0% w/w, about 2.5% w/w, or about 2.0% w/w (inclusive); about 2.0% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 9.5% w/w, about 9.0% w/w, about 8.5% w/w, about 8.0% w/w, about 7.5% w/w, about 7.0% w/w, about 6.5% w/w, about 6.0% w/w, about 5.5% w/w, about 5.0% w/w, about 4.5% w/w, about 4.0% w/w, about 3.5% w/w, about 3.0% w/w, or about 2.5% w/w (inclusive); about 2.5% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 9.5% w/w, about 9.0% w/w, about 8.5% w/w, about 8.0% w/w, about 7.5% w/w, about 7.0% w/w, about 6.5% w/w, about 6.0% w/w, about 5.5% w/w, about 5.0% w/w, about 4.5% w/w, about 4.0% w/w, about 3.5% w/w, or about 3.0% w/w (inclusive); about 3.0% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 9.5% w/w, about 9.0% w/w, about 8.5% w/w, about 8.0% w/w, about 7.5% w/w, about 7.0% w/w, about 6.5% w/w, about 6.0% w/w, about 5.5% w/w, about 5.0% w/w, about 4.5% w/w, about 4.0% w/w, or about 3.5% w/w, inclusive; about 3.5% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 9.5% w/w, about 9.0% w/w, about 8.5% w/w, about 8.0% w/w, about 7.5% w/w, about 7.0% w/w, about 6.5% w/w, about 6.0% w/w, about 5.5% w/w, about 5.0% w/w, about 4.5% w/w, or about 4.0% w/w (inclusive); about 4.0% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 9.5% w/w, about 9.0% w/w, about 8.5% w/w, about 8.0% w/w, about 7.5% w/w, about 7.0% w/w, about 6.5% w/w, about 6.0% w/w, about 5.5% w/w, about 5.0% w/w, or about 4.5% w/w, inclusive; about 4.5% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 9.5% w/w, about 9.0% w/w, about 8.5% w/w, about 8.0% w/w, about 7.5% w/w, about 7.0% w/w, about 6.5% w/w, about 6.0% w/w, about 5.5% w/w, or about 5.0% w/w, inclusive; about 5.0% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 9.5% w/w, about 9.0% w/w, about 8.5% w/w, about 8.0% w/w, about 7.5% w/w, about 7.0% w/w, about 6.5% w/w, about 6.0% w/w, or about 5.5% w/w, inclusive; about 5.5% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 9.5% w/w, about 9.0% w/w, about 8.5% w/w, about 8.0% w/w, about 7.5% w/w, about 7.0% w/w, about 6.5% w/w, or about 6.0% w/w, inclusive; about 6.0% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 9.5% w/w, about 9.0% w/w, about 8.5% w/w, about 8.0% w/w, about 7.5% w/w, about 7.0% w/w, or about 6.5% w/w (inclusive); about 6.5% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 9.5% w/w, about 9.0% w/w, about 8.5% w/w, about 8.0% w/w, about 7.5% w/w, or about 7.0% w/w (inclusive); about 7.0% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 9.5% w/w, about 9.0% w/w, about 8.5% w/w, about 8.0% w/w, or about 7.5% w/w, inclusive; about 7.5% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 9.5% w/w, about 9.0% w/w, about 8.5% w/w, or about 8.0% w/w, inclusive; about 8.0% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 9.5% w/w, about 9.0% w/w, or about 8.5% w/w, inclusive; about 8.5% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 9.5% w/w, or about 9.0% w/w, inclusive; about 9.0% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, or about 9.5% w/w, inclusive; about 9.5% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, or about 10% w/w, inclusive; about 10% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, or about 12% w/w, inclusive; about 12% to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, or about 14% w/w, inclusive; about 14% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, or about 16% w/w, inclusive; about 16% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, or about 18% w/w, inclusive; about 18% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, or about 20% w/w, inclusive; about 20% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, or about 22% w/w, inclusive; about 22% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, or about 24% w/w, inclusive; about 24% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, or about 26% w/w, inclusive; about 26% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, or about 28% w/w, inclusive; about 28% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, or about 30% w/w, inclusive; about 30% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, or about 32% w/w, inclusive; about 32% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, or about 34% w/w, inclusive; about 34% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, or about 36% w/w, inclusive; about 36% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, about 40% w/w, or about 38% w/w, inclusive; about 38% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, about 42% w/w, or about 40% w/w, inclusive; about 40% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, about 44% w/w, or about 42% w/w, inclusive; about 42% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, about 46% w/w, or about 44% w/w, inclusive; about 44% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, about 48% w/w, or about 46% w/w, inclusive; about 46% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, about 50% w/w, or about 48% w/w, inclusive; about 48% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, about 52% w/w, or about 50% w/w, inclusive; about 50% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, about 54% w/w, or about 52% w/w, inclusive; about 52% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, about 56% w/w, or about 54% w/w, inclusive; about 54% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, about 58% w/w, or about 56% w/w, inclusive; about 56% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, about 60% w/w, or about 58% w/w, inclusive; about 58% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, about 62% w/w, or about 60% w/w, inclusive; about 60% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, about 64% w/w, or about 62% w/w, inclusive; about 62% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, about 66% w/w, or about 64% w/w, inclusive; about 64% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, about 68% w/w, or about 66% w/w, inclusive; about 66% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, about 70% w/w, or about 68% w/w, inclusive; about 68% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, about 72% w/w, or about 70% w/w, inclusive; about 70% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, about 74% w/w, or about 72% w/w, inclusive; about 72% w/w to about 95% w/w, about 94% w/w, about 92% w/w, about 90% w/w, about 88% w/w, about 86% w/w, about 84% w/w, about 82% w/w, about 80% w/w, about 78% w/w, about 76% w/w, or about 74% w/w, inclusive; about 74% w/w to about 95% w/w, about 94% w/w, about 92% w/w, about 90% w/w, about 88% w/w, about 86% w/w, about 84% w/w, about 82% w/w, about 80% w/w, about 78% w/w, or about 76% w/w, inclusive; about 76% w/w to about 95% w/w, about 94% w/w, about 92% w/w, about 90% w/w, about 88% w/w, about 86% w/w, about 84% w/w, about 82% w/w, about 80% w/w, or about 78% w/w, inclusive; about 78% w/w to about 95% w/w, about 94% w/w, about 92% w/w, about 90% w/w, about 88% w/w, about 86% w/w, about 84% w/w, about 82% w/w, or about 80% w/w, inclusive; about 80% w/w to about 95% w/w, about 94% w/w, about 92% w/w, about 90% w/w, about 88% w/w, about 86% w/w, about 84% w/w, or about 82% w/w, inclusive; about 82% w/w to about 95% w/w, about 94% w/w, about 92% w/w, about 90% w/w, about 88% w/w, about 86% w/w, or about 84% w/w, inclusive; about 84% w/w to about 95% w/w, about 94% w/w, about 92% w/w, about 90% w/w, about 88% w/w, or about 86% w/w, inclusive; about 86% w/w to about 95% w/w, about 94% w/w, about 92% w/w, about 90% w/w, or about 88% w/w (inclusive); about 88% w/w to about 95% w/w, about 94% w/w, about 92% w/w, or about 90% w/w, inclusive; about 90% w/w to about 95% w/w, about 94% w/w, or about 92% w/w, inclusive; about 92% w/w to about 95% w/w or about 94% w/w, inclusive; or about 94% w/w to about 95% w/w, inclusive, of the composition is a zeolite or the sum total of two or more zeolites.

In some examples of any of the compositions provided herein, at least about 10% w/w, at least about 12% w/w, at least about 14% w/w, at least about 15% w/w, at least about 16% w/w, at least about 18% w/w, at least about 20% w/w, at least about 22% w/w, at least about 24% w/w, at least about 25% w/w, at least about 26% w/w, at least about 28% w/w, at least about 30% w/w, at least about 32% w/w, at least about 34% w/w, at least about 36% w/w, at least about 38% w/w, at least about 40% w/w, at least about 42% w/w, at least about 44% w/w, at least about 45% w/w, at least about 46% w/w, at least about 48% w/w, at least about 50% w/w, at least about 52% w/w, at least about 54% w/w, at least about 55% w/w, at least about 56% w/w, at least about 58% w/w, at least about 60% w/w, at least about 62% w/w, at least about 64% w/w, at least about 65% w/w, at least about 66% w/w, at least about 68% w/w, at least about 70% w/w, at least about 72% w/w, at least about 74% w/w, at least about 75% w/w, at least about 76% w/w, at least about 78% w/w, at least about 80% w/w, at least about 82% w/w, at least about 80% w/w, at least about 82% w/w, at least about 84% w/w, at least about 85% w/w, at least about 86% w/w, at least about 88% w/w, at least about 90% w/w, at least about 92% w/w, at least about 94% w/w, at least about 95% w/w, or at least about 96% w/w, of the composition is a zeolite or the sum total of two or more zeolites.

In some examples of any of the compositions provided herein, about 0.01% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, about 3.4% w/w, about 3.2% w/w, about 3.0% w/w, about 2.8% w/w, about 2.6% w/w, about 2.4% w/w, about 2.2% w/w, about 2.0% w/w, about 1.8% w/w, about 1.6% w/w, about 1.4% w/w, about 1.2% w/w, about 1.0% w/w, about 0.8% w/w, about 0.6% w/w, about 0.5% w/w, about 0.4% w/w, about 0.2% w/w, or about 0.1% w/w, inclusive; about 0.1% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, about 3.4% w/w, about 3.2% w/w, about 3.0% w/w, about 2.8% w/w, about 2.6% w/w, about 2.4% w/w, about 2.2% w/w, about 2.0% w/w, about 1.8% w/w, about 1.6% w/w, about 1.4% w/w, about 1.2% w/w, about 1.0% w/w, about 0.8% w/w, about 0.6% w/w, about 0.5% w/w, about 0.4% w/w, or about 0.2% w/w, inclusive; about 0.2% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, about 3.4% w/w, about 3.2% w/w, about 3.0% w/w, about 2.8% w/w, about 2.6% w/w, about 2.4% w/w, about 2.2% w/w, about 2.0% w/w, about 1.8% w/w, about 1.6% w/w, about 1.4% w/w, about 1.2% w/w, about 1.0% w/w, about 0.8% w/w, about 0.6% w/w, about 0.5% w/w, or about 0.4% w/w, inclusive; about 0.4% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, about 3.4% w/w, about 3.2% w/w, about 3.0% w/w, about 2.8% w/w, about 2.6% w/w, about 2.4% w/w, about 2.2% w/w, about 2.0% w/w, about 1.8% w/w, about 1.6% w/w, about 1.4% w/w, about 1.2% w/w, about 1.0% w/w, about 0.8% w/w, about 0.6% w/w, or about 0.5% w/w, inclusive; about 0.5% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, about 3.4% w/w, about 3.2% w/w, about 3.0% w/w, about 2.8% w/w, about 2.6% w/w, about 2.4% w/w, about 2.2% w/w, about 2.0% w/w, about 1.8% w/w, about 1.6% w/w, about 1.4% w/w, about 1.2% w/w, about 1.0% w/w, about 0.8% w/w, or about 0.6% w/w, inclusive; about 0.6% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, about 3.4% w/w, about 3.2% w/w, about 3.0% w/w, about 2.8% w/w, about 2.6% w/w, about 2.4% w/w, about 2.2% w/w, about 2.0% w/w, about 1.8% w/w, about 1.6% w/w, about 1.4% w/w, about 1.2% w/w, about 1.0% w/w, or about 0.8% w/w, inclusive; about 0.8% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, about 3.4% w/w, about 3.2% w/w, about 3.0% w/w, about 2.8% w/w, about 2.6% w/w, about 2.4% w/w, about 2.2% w/w, about 2.0% w/w, about 1.8% w/w, about 1.6% w/w, about 1.4% w/w, about 1.2% w/w, or about 1.0% w/w, inclusive; about 1.0% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, about 3.4% w/w, about 3.2% w/w, about 3.0% w/w, about 2.8% w/w, about 2.6% w/w, about 2.4% w/w, about 2.2% w/w, about 2.0% w/w, about 1.8% w/w, about 1.6% w/w, about 1.4% w/w, or about 1.2% w/w, inclusive; about 1.2% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, about 3.4% w/w, about 3.2% w/w, about 3.0% w/w, about 2.8% w/w, about 2.6% w/w, about 2.4% w/w, about 2.2% w/w, about 2.0% w/w, about 1.8% w/w, about 1.6% w/w, or about 1.4% w/w, inclusive; about 1.4% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, about 3.4% w/w, about 3.2% w/w, about 3.0% w/w, about 2.8% w/w, about 2.6% w/w, about 2.4% w/w, about 2.2% w/w, about 2.0% w/w, about 1.8% w/w, or about 1.6% w/w, inclusive; about 1.6% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, about 3.4% w/w, about 3.2% w/w, about 3.0% w/w, about 2.8% w/w, about 2.6% w/w, about 2.4% w/w, about 2.2% w/w, about 2.0% w/w, or about 1.8% w/w, inclusive; about 1.8% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, about 3.4% w/w, about 3.2% w/w, about 3.0% w/w, about 2.8% w/w, about 2.6% w/w, about 2.4% w/w, about 2.2% w/w, or about 2.0% w/w, inclusive; about 2.0% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, about 3.4% w/w, about 3.2% w/w, about 3.0% w/w, about 2.8% w/w, about 2.6% w/w, about 2.4% w/w, or about 2.2% w/w, inclusive; about 2.2% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, about 3.4% w/w, about 3.2% w/w, about 3.0% w/w, about 2.8% w/w, about 2.6% w/w, or about 2.4% w/w, inclusive; about 2.4% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, about 3.4% w/w, about 3.2% w/w, about 3.0% w/w, about 2.8% w/w, or about 2.6% w/w, inclusive; about 2.6% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, about 3.4% w/w, about 3.2% w/w, about 3.0% w/w, or about 2.8% w/w, inclusive; about 2.8% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, about 3.4% w/w, about 3.2% w/w, or about 3.0% w/w, inclusive; about 3.0% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, about 3.4% w/w, or about 3.2% w/w, inclusive; about 3.2% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, about 3.6% w/w, or about 3.4% w/w, inclusive; about 3.4% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, about 3.8% w/w, or about 3.6% w/w, inclusive; about 3.6% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, or about 3.8% w/w, inclusive; about 3.8% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, about 4.2% w/w, about 4.0% w/w, inclusive; about 4.0% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, about 4.4% w/w, or about 4.2% w/w, inclusive; about 4.2% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, about 4.6% w/w, or about 4.4% w/w, inclusive; about 4.4% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, about 4.8% w/w, or about 4.6% w/w, inclusive; about 4.6% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, about 5.0% w/w, or about 4.8% w/w, inclusive; about 4.8% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, about 5.2% w/w, or about 5.0% w/w, inclusive; about 5.0% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, about 5.4% w/w, or about 5.2% w/w, inclusive; about 5.2% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, about 5.6% w/w, or about 5.4% w/w, inclusive; about 5.4% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, about 5.8% w/w, or about 5.6% w/w, inclusive; about 5.6% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, about 6.0% w/w, or about 5.8% w/w, inclusive; about 5.8% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, about 6.2% w/w, or about 6.0% w/w, inclusive; about 6.0% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, about 6.4% w/w, or about 6.2% w/w, inclusive; about 6.2% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, about 6.6% w/w, or about 6.4% w/w, inclusive; about 6.4% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, about 6.8% w/w, or about 6.6% w/w, inclusive; about 6.6% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, about 7.0% w/w, or about 6.8% w/w, inclusive; about 6.8% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, about 7.2% w/w, or about 7.0% w/w, inclusive; about 7.0% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, about 7.4% w/w, or about 7.2% w/w, inclusive; about 7.2% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, about 7.6% w/w, or about 7.4% w/w, inclusive; about 7.4% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, about 7.8% w/w, or about 7.6% w/w, inclusive; about 7.6% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, about 8.0% w/w, or about 7.8% w/w, inclusive; about 7.8% to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, about 8.2% w/w, or about 8.0% w/w, inclusive; about 8.0% w/w to about 10.0% w/w, about 9.8% w/w, about 9.6% w/w, about 9.4% w/w, about 9.2% w/w, about 9.0% w/w, about 8.8% w/w, about 8.6% w/w, about 8.4% w/w, or about 8.2% w/w, inclusive; about 8.2% w/w to about 10.0% w/w, about 9.8% w/w, about 9.6% w/w, about 9.4% w/w, about 9.2% w/w, about 9.0% w/w, about 8.8% w/w, about 8.6% w/w, or about 8.4% w/w, inclusive; about 8.4% w/w to about 10.0% w/w, about 9.8% w/w, about 9.6% w/w, about 9.4% w/w, about 9.2% w/w, about 9.0% w/w, about 8.8% w/w, or about 8.6% w/w, inclusive; about 8.6% w/w to about 10.0% w/w, about 9.8% w/w, about 9.6% w/w, about 9.4% w/w, about 9.2% w/w, about 9.0% w/w, or about 8.8% w/w, inclusive; about 8.8% w/w to about 10.0% w/w, about 9.8% w/w, about 9.6% w/w, about 9.4% w/w, about 9.2% w/w, or about 9.0% w/w, inclusive; about 9.0% w/w to about 10.0% w/w, about 9.8% w/w, about 9.6% w/w, about 9.4% w/w, or about 9.2% w/w, inclusive; about 9.2% w/w to about 10.0% w/w, about 9.8% w/w, about 9.6% w/w, or about 9.4% w/w, inclusive; about 9.4% w/w to about 10.0% w/w, about 9.8% w/w, or about 9.6% w/w, inclusive; about 9.6% w/w to about 10.0% w/w or about 9.8% w/w, inclusive; or about 9.8% w/w to about 10.0% w/w, inclusive, of the composition provided herein is a zeolite or the sum total of two or more zeolites.

In some examples of any of the compositions provided herein, at least 10% w/w, at least about 12% w/w, at least about 14% w/w, at least about 15% w/w, at least about 16% w/w, at least about 18% w/w, at least about 20% w/w, at least about 22% w/w, at least about 24% w/w, at least about 25% w/w, at least about 26% w/w, at least about 28% w/w, at least about 30% w/w, at least about 32% w/w, at least about 34% w/w, at least about 35% w/w, at least about 36% w/w, at least about 38% w/w, at least about 40% w/w, at least about 42% w/w, at least about 44% w/w, at least about 45% w/w, at least about 46% w/w, at least about 48% w/w, at least about 50% w/w, at least about 52% w/w, at least about 54% w/w, at least about 55% w/w, at least 56% w/w, at least about 58% w/w, at least about 60% w/w, at least about 62% w/w, at least about 64% w/w, at least about 65% w/w, at least about 66% w/w, at least about 68% w/w, at least about 70% w/w, at least about 72% w/w, at least about 74% w/w, at least about 76% w/w, at least about 78% w/w, at least about 80% w/w, at least about 82% w/w, at least about 84% w/w, at least about 85% w/w, at least about 86% w/w, at least about 88% w/w, or at least about 90% w/w of a composition provided herein is a zeolite or the sum total of two or more zeolites. In some examples of any of the compositions provided herein, no more than 10% w/w, no more than 12% w/w, no more than 14% w/w, no more than 15% w/w, no more than 16% w/w, no more than 18% w/w, no more than 20% w/w, no more than 22% w/w, no more than 24% w/w, no more than 25% w/w, no more than 26% w/w, no more than 28% w/w, no more than 30% w/w, no more than 32% w/w, no more than 34% w/w, no more than 35% w/w, no more than 36% w/w, no more than 38% w/w, no more than 40% w/w, no more than 42% w/w, no more than 44% w/w, no more than 45% w/w, no more than 46% w/w, no more than 48% w/w, no more than 50% w/w, no more than 52% w/w, no more than 54% w/w, no more than 55% w/w, no more than 56% w/w, no more than 58% w/w, no more than 60% w/w, no more than 62% w/w, no more than 64% w/w, no more than 65% w/w, no more than 66% w/w, no more than 68% w/w, no more than 70% w/w, no more than 72% w/w, no more than 74% w/w, no more than 75% w/w, no more than 76% w/w, no more than 78% w/w, no more than 80% w/w, no more than 82% w/w, no more than 84% w/w, no more than 85% w/w, no more than 86% w/w, no more than 88% w/w, or no more than 90% w/w of a composition provided herein is a zeolite or the sum total of two or more zeolites.

In some examples, a single dose of any of the compositions provided herein can include from about 1 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 150 mg, about 100 mg, about 50 mg, about 20 mg, or about 10 mg, inclusive; about 10 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 150 mg, about 100 mg, about 50 mg, or about 20 mg, inclusive; about 20 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 150 mg, about 100 mg, or about 50 mg, inclusive; about 50 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 150 mg, or about 100 mg, inclusive; about 100 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, or about 150 mg, inclusive; about 150 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, or about 200 mg, inclusive; about 200 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, or about 250 mg, inclusive; about 250 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, or about 300 mg, inclusive; about 300 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, or about 350 mg, inclusive; about 350 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, or about 400 mg, inclusive; about 400 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, or about 450 mg, inclusive; about 450 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, or about 500 mg, inclusive; about 500 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, or about 550 mg, inclusive; about 550 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, or about 600 mg, inclusive; about 600 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, or about 650 mg, inclusive; about 650 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, or about 700 mg, inclusive; about 700 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, or about 750 mg, inclusive; about 750 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, or about 800 mg, inclusive; about 800 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, or about 850 mg, inclusive; about 850 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, or about 900 mg, inclusive; about 900 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, or about 950 mg, inclusive; about 950 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, or about 1,000 mg, inclusive; about 1,000 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, or about 1,100 mg, inclusive; about 1,100 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, or about 1,200 mg, inclusive; about 1,200 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, or about 1,300 mg, inclusive; about 1,300 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, or about 1,400 mg, inclusive; about 1,400 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, or about 1,500 mg, inclusive; about 1,500 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, or about 1,600 mg, inclusive; about 1,600 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, or about 1,700 mg, inclusive; about 1,700 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, or about 1,800 mg, inclusive; about 1,800 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, or about 1,900 mg, inclusive; about 1,900 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, or about 2,000 mg, inclusive; about 2,000 mg to about 3,000 mg, about 2,900 mg, about 2,800 mg, about 2,700 mg, about 2,600 mg, about 2,500 mg, about 2,400 mg, about 2,300 mg, about 2,200 mg, or about 2,100 mg, inclusive; about 2,100 mg to about 3,000 mg, about 2,900 mg, about 2,800 mg, about 2,700 mg, about 2,600 mg, about 2,500 mg, about 2,400 mg, about 2,300 mg, or about 2,200 mg, inclusive; about 2,200 mg to about 3,000 mg, about 2,900 mg, about 2,800 mg, about 2,700 mg, about 2,600 mg, about 2,500 mg, about 2,400 mg, or about 2,300 mg, inclusive; about 2,300 mg to about 3,000 mg, about 2,900 mg, about 2,800 mg, about 2,700 mg, about 2,600 mg, about 2,500 mg, or about 2,400 mg, inclusive; about 2,400 mg to about 3,000 mg, about 2,900 mg, about 2,800 mg, about 2,700 mg, about 2,600 mg, or about 2,500 mg, inclusive; about 2,500 mg to about 3,000 mg, about 2,900 mg, about 2,800 mg, about 2,700 mg, or about 2,600 mg, inclusive; about 2,600 mg to about 3,000 mg, about 2,900 mg, about 2,800 mg, or about 2,700 mg, inclusive; about 2,700 mg to about 3,000 mg, about 2,900 mg, or about 2,800 mg, inclusive; about 2,800 mg to about 3,000 mg or about 2,900 mg, inclusive; or about 2,900 mg to about 3,000 mg, inclusive, of a zeolite or of each zeolite in a composition including two or more zeolites.

In some examples, a composition provided herein can be formulated to contain an amount of a zeolite such that a daily dose of about 0.1 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 150 mg, about 100 mg, about 50 mg, about 20 mg, about 10 mg, about 5 mg, or about 1 mg, inclusive; about 1 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 150 mg, about 100 mg, about 50 mg, about 20 mg, about 10 mg, or about 5 mg, inclusive; about 5 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 150 mg, about 100 mg, about 50 mg, about 20 mg, or about 10 mg, inclusive; about 10 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 150 mg, about 100 mg, about 50 mg, or about 20 mg, inclusive; about 20 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 150 mg, about 100 mg, or about 50 mg, inclusive; about 50 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 150 mg, or about 100 mg, inclusive; about 100 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, or about 150 mg, inclusive; about 150 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, or about 200 mg, inclusive; about 200 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, or about 250 mg, inclusive; about 250 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, or about 300 mg, inclusive; about 300 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, about 400 mg, or about 350 mg, inclusive; about 350 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, about 450 mg, or about 400 mg, inclusive; about 400 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, about 500 mg, or about 450 mg, inclusive; about 450 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, about 550 mg, or about 500 mg, inclusive; about 500 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, about 600 mg, or about 550 mg, inclusive; about 550 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, about 650 mg, or about 600 mg, inclusive; about 600 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, about 700 mg, or about 650 mg, inclusive; about 650 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, about 750 mg, or about 700 mg, inclusive; about 700 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, about 800 mg, or about 750 mg, inclusive; about 750 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, about 850 mg, or about 800 mg, inclusive; about 800 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, about 900 mg, or about 850 mg, inclusive; about 850 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, about 950 mg, or about 900 mg, inclusive; about 900 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, about 1,000 mg, or about 950 mg, inclusive; about 950 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, about 1,100 mg, or about 1,000 mg, inclusive; about 1,000 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, about 1,200 mg, or about 1,100 mg, inclusive; about 1,100 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, about 1,300 mg, or about 1,200 mg, inclusive; about 1,200 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, about 1,400 mg, or about 1,300 mg, inclusive; about 1,300 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, about 1,600 mg, about 1,500 mg, or about 1,400 mg, inclusive; about 1,400 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, about 1,700 mg, or about 1,600 mg, inclusive; about 1,600 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, about 1,800 mg, or about 1,700 mg, inclusive; about 1,700 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, about 1,900 mg, or about 1,800 mg, inclusive; about 1,800 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, about 2,000 mg, or about 1,900 mg, inclusive; about 1,900 mg to about 3,000 mg, about 2,900 mg, about 2,800 mg, about 2,700 mg, about 2,600 mg, about 2,500 mg, about 2,400 mg, about 2,300 mg, about 2,200 mg, about 2,100 mg, or about 2,000 mg, inclusive; about 2,000 mg to about 3,000 mg, about 2,900 mg, about 2,800 mg, about 2,700 mg, about 2,600 mg, about 2,500 mg, about 2,400 mg, about 2,300 mg, about 2,200 mg, or about 2,100 mg, inclusive; about 2,100 mg to about 3,000 mg, about 2,900 mg, about 2,800 mg, about 2,700 mg, about 2,600 mg, about 2,500 mg, about 2,400 mg, about 2,300 mg, or about 2,200 mg, inclusive; about 2,200 mg to about 3,000 mg, about 2,900 mg, about 2,800 mg, about 2,700 mg, about 2,600 mg, about 2,500 mg, about 2,400 mg, or about 2,300 mg, inclusive; about 2,300 mg to about 3,000 mg, about 2,900 mg, about 2,800 mg, about 2,700 mg, about 2,600 mg, about 2,500 mg, or about 2,400 mg, inclusive; about 2,400 mg to about 3,000 mg, about 2,900 mg, about 2,800 mg, about 2,700 mg, about 2,600 mg, or about 2,500 mg, inclusive; about 2,500 mg to about 3,000 mg, about 2,900 mg, about 2,800 mg, about 2,700 mg, or about 2,600 mg, inclusive; about 2,600 mg to about 3,000 mg, about 2,900 mg, about 2,800 mg, or about 2,700 mg, inclusive; about 2,700 mg to about 3,000 mg, about 2,900 mg, or about 2,800 mg, inclusive; about 2,800 mg to about 3,000 mg or about 2,900 mg, inclusive; or about 2,900 mg to about 3,000 mg, inclusive, of a zeolite or of each zeolite in a composition including two or more zeolites can be administered to a subject (e.g., any of the subjects described herein), e.g., in a 24-hour period.

In some examples, a unit dose can include about or at least 1 mg, about or at least 10 mg, about or at least 20 mg, about or at least 50 mg, about or at least 100 mg, about or at least 150 mg, about or at least 200 mg, about or at least 250 mg, about or at least 300 mg, about or at least 350 mg, about or at least 400 mg, about or at least 450 mg, about or at least 500 mg, about or at least 550 mg, about or at least 600 mg, about or at least 650 mg, about or at least 700 mg, about or at least 750 mg, about or at least 800 mg, about or at least 850 mg, about or at least 900 mg, about or at least 950 mg, about or at least 1,000 mg, about or at least 1,100 mg, about or at least 1,200 mg, about or at least 1,300 mg, about or at least 1,400 mg, about or at least 1,500 mg, about or at least 1,600 mg, about or at least 1,700 mg, about or at least 1,800 mg, about or at least 1,900 mg, about or at least 2,000 mg, about or at least 2,100 mg, about or at least 2,200 mg, about or at least 2,300 mg, about or at least 2,400 mg, about or at least 2,500 mg, about or at least 2,600 mg, about or at least 2,700 mg, about or at least 2,800 mg, about or at least 2,900 mg, or about or at least 3,000 mg of a zeolite or each zeolite in compositions that include two or more zeolites. In some examples, a dose can include less than about 1 mg, less than about 10 mg, less than about 20 mg, less than about 50 mg, less than about 100 mg, less than about 150 mg, less than about 200 mg, less than about 250 mg, less than about 300 mg, less than about 350 mg, less than about 400 mg, less than about 450 mg, less than about 500 mg, less than about 550 mg, less than about 600 mg, less than about 650 mg, less than about 700 mg, less than about 750 mg, less than about 800 mg, less than about 850 mg, less than about 900 mg, less than about 950 mg, less than about 1,000 mg, less than about 1,100 mg, less than about 1,200 mg, less than about 1,300 mg, less than about 1,400 mg, less than about 1,500 mg, less than about 1,600 mg, less than about 1,700 mg, less than about 1,800 mg, less than about 1,900 mg, less than about 2,000 mg, less than about 2,100 mg, less than about 2,200 mg, less than about 2,300 mg, less than about 2,400 mg, less than about 2,500 mg, less than about 2,600 mg, less than about 2,700 mg, less than about 2,800 mg, less than about 2,900 mg, or less than about 3,000 mg of a zeolite or each zeolite in compositions that include two or more zeolites.

A composition described herein can include a zeolite (e.g., one or more of any of the zeolites described herein) and one or more additional active agents selected from the group of: riboflavin, a colonic acidifier, an ammonia reducer, an ammonia sequestering agent, an intestinal deodorizer, and a probiotic.

Any of the compositions provided herein can further include riboflavin (e.g., a therapeutically effective amount of riboflavin), which also known as Vitamin B2, vitamin G, and lactoflavin. Riboflavin may be used to reduce trimethylamine excretion-based body odor. Riboflavin may be used to increase residual activity of a liver flavoprotein known as flavin containing mono-oxygenase (FMO3) in a subject with primary TMAU. In some examples, riboflavin may provide reduced trimethylamine excretion in subjects ingesting the compositions provided herein. Riboflavin may be obtained from a fermentation process of any number of bacteria, or through a combination of synthetic and biotechnology techniques described in the art (see, e.g., U.S. Pat. Nos. 7,427,619, 6,150,364, and 5,589,355). The biotechnology techniques for producing riboflavin may use various microorganisms, including filamentous fungi such as Ashbya gossypii, Candida famata and Candida flaveri, and bacteria Corynebacterium ammoniagenes and Bacillus subtilis.

Any of the compositions provided herein can include a therapeutically effective amount of riboflavin. In some examples of any of the compositions provided herein, about 0.1% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, about 1% w/w, or about 0.5% w/w, inclusive; about 0.2% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, about 1% w/w, or about 0.5% w/w, inclusive; about 0.5% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, or about 1% w/w, inclusive; about 1% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, or about 2% w/w, inclusive; about 2% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, or about 3% w/w, inclusive; about 3% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, or about 4% w/w, inclusive; about 4% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, or about 5% w/w inclusive; about 5% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, or about 6% w/w, inclusive; about 6% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, or about 8% w/w, inclusive; about 8% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, or about 10% w/w, inclusive; about 10% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, or about 12% w/w, inclusive; about 12% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, or about 14% w/w, inclusive; about 14% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, or about 16% w/w, inclusive; about 16% to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, or about 18% w/w, inclusive; about 18% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, or about 20% w/w, inclusive; about 20% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, or about 22% w/w, inclusive; about 22% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, or about 24% w/w, inclusive; about 24% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, or about 26% w/w, inclusive; about 26% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, or about 28% w/w, inclusive; about 28% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, or about 30% w/w, inclusive; about 30% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, or about 32% w/w, inclusive; about 32% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, inclusive; about 34% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, or about 36% w/w, inclusive; about 36% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, or about 38% w/w, inclusive; about 38% w/w to about 60% w/w, about 58% w/w, about 56% w/w, about 54% w/w, about 52% w/w, about 50% w/w, about 48% w/w, about 46% w/w, about 44% w/w, about 42% w/w, or about 40% w/w, inclusive; about 40% w/w to about 60% w/w, about 58% w/w, about 56% w/w, about 54% w/w, about 52% w/w, about 50% w/w, about 48% w/w, about 46% w/w, about 44% w/w, or about 42% w/w, inclusive; about 42% w/w to about 60% w/w, about 58% w/w, about 56% w/w, about 54% w/w, about 52% w/w, about 50% w/w, about 48% w/w, about 46% w/w, or about 44% w/w, inclusive; about 44% w/w to about 60% w/w, about 58% w/w, about 56% w/w, about 54% w/w, about 52% w/w, about 50% w/w, about 48% w/w, or about 46% w/w, inclusive; about 46% w/w to about 60% w/w, about 58% w/w, about 56% w/w, about 54% w/w, about 52% w/w, about 50% w/w, or about 48% w/w, inclusive; about 48% w/w to about 60% w/w, about 58% w/w, about 56% w/w, about 54% w/w, about 52% w/w, or about 50% w/w, inclusive; about 50% w/w to about 60% w/w, about 58% w/w, about 56% w/w, about 54% w/w, or about 52% w/w, inclusive; about 52% w/w to about 60% w/w, about 58% w/w, about 56% w/w, or about 54% w/w, inclusive; about 54% w/w to about 60% w/w, about 58% w/w, or about 56% w/w, inclusive; about 56% w/w to about 60% w/w, or about 58% w/w, inclusive; or about 58% w/w to about 60% w/w, inclusive, of the composition is riboflavin.

In some embodiments, no more than 0.5% w/w, no more than 1% w/w, no more than 2% w/w, no more than 4% w/w, no more than 5% w/w, no more than 6% w/w, no more than 8% w/w, no more than 10% w/w, no more than 12% w/w, no more than 14% w/w, no more than 15% w/w, no more than 16% w/w, no more than 18% w/w, no more than 20% w/w, no more than 22% w/w, no more than 24% w/w, no more than 25% w/w, no more than 26% w/w, no more than 28% w/w, no more than 30% w/w, no more than 32% w/w, no more than 34% w/w, no more than 35% w/w, no more than 36% w/w, no more than 38% w/w, no more than 40% w/w, no more than 42% w/w, no more than 44% w/w, no more than 45% w/w, no more than 46% w/w, no more than 48% w/w, no more than 50% w/w, no more than 52% w/w, no more than 54% w/w, no more than 55% w/w, no more than 56% w/w, no more than 58% w/w, or no more than 60% w/w, of any of the compositions described herein is riboflavin.

In some examples, a single dose of any of the compositions provided herein can include from about 0.1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, about 1 mg, or about 0.5 mg, inclusive; about 0.5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg, inclusive; about 1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, or about 5 mg, inclusive; about 5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, or about 10 mg, inclusive; about 10 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, or about 20 mg, inclusive; about 20 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, or about 40 mg, inclusive; about 40 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, or about 60 mg, inclusive; about 60 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, or about 80 mg, inclusive; about 80 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, or about 100 mg, inclusive; about 100 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, or about 150 mg, inclusive; about 150 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, or about 200 mg, inclusive; about 200 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, or about 250 mg, inclusive; about 250 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, or about 300 mg, inclusive; about 300 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, or about 350 mg, inclusive; about 350 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, or about 400 mg, inclusive; about 400 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, or about 450 mg, inclusive; about 450 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, or about 500 mg, inclusive; about 500 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, or about 600 mg, inclusive; about 600 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, or about 700 mg, inclusive; about 700 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, or about 800 mg, inclusive; about 800 mg to about 1,800 mg, about 1,700 mg, about 1,600 mg, about 1,500 mg, about 1,400 mg, about 1,300 mg, about 1,200 mg, about 1,100 mg, about 1,000 mg, or about 900 mg, inclusive; about 900 mg to about 1,800 mg, about 1,700 mg, about 1,600 mg, about 1,500 mg, about 1,400 mg, about 1,300 mg, about 1,200 mg, about 1,100 mg, or about 1,000 mg, inclusive; about 1,000 mg to about 1,800 mg, about 1,700 mg, about 1,600 mg, about 1,500 mg, about 1,400 mg, about 1,300 mg, about 1,200 mg, or about 1,100 mg, inclusive; about 1,100 mg to about 1,800 mg, about 1,700 mg, about 1,600 mg, about 1,500 mg, about 1,400 mg, about 1,300 mg, about 1,200 mg, inclusive; about 1,200 mg to about 1,800 mg, about 1,700 mg, about 1,600 mg, about 1,500 mg, about 1,400 mg, or about 1,300 mg, inclusive; about 1,300 mg to about 1,800 mg, about 1,700 mg, about 1,600 mg, about 1,500 mg, or about 1,400 mg, inclusive; about 1,400 mg to about 1,800 mg, about 1,700 mg, about 1,600 mg, or about 1,500 mg, inclusive; about 1,500 mg to about 1,800 mg, about 1,700 mg, or about 1,600 mg, inclusive; about 1,600 mg to about 1,800 mg, or about 1,700 mg, inclusive; or about 1,700 mg to about 1,800 mg, inclusive, of riboflavin.

In some examples, a composition provided herein can be formulated to contain an amount of riboflavin such that a daily dose of about 0.1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, about 1 mg, or about 0.5 mg, inclusive; about 0.5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg, inclusive; about 1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, or about 5 mg, inclusive; about 5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, or about 10 mg, inclusive; about 10 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, or about 20 mg, inclusive; about 20 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, or about 40 mg, inclusive; about 40 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, or about 60 mg, inclusive; about 60 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, or about 80 mg, inclusive; about 80 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, or about 100 mg, inclusive; about 100 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, or about 150 mg, inclusive; about 150 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, or about 200 mg, inclusive; about 200 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, or about 250 mg, inclusive; about 250 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, or about 300 mg, inclusive; about 300 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, or about 350 mg, inclusive; about 350 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, or about 400 mg, inclusive; about 400 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, or about 450 mg, inclusive; about 450 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, or about 500 mg, inclusive; about 500 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, or about 600 mg, inclusive; about 600 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, or about 700 mg, inclusive; about 700 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, or about 800 mg, inclusive; about 800 mg to about 1,800 mg, about 1,700 mg, about 1,600 mg, about 1,500 mg, about 1,400 mg, about 1,300 mg, about 1,200 mg, about 1,100 mg, about 1,000 mg, or about 900 mg, inclusive; about 900 mg to about 1,800 mg, about 1,700 mg, about 1,600 mg, about 1,500 mg, about 1,400 mg, about 1,300 mg, about 1,200 mg, about 1,100 mg, or about 1,000 mg, inclusive; about 1,000 mg to about 1,800 mg, about 1,700 mg, about 1,600 mg, about 1,500 mg, about 1,400 mg, about 1,300 mg, about 1,200 mg, or about 1,100 mg, inclusive; about 1,100 mg to about 1,800 mg, about 1,700 mg, about 1,600 mg, about 1,500 mg, about 1,400 mg, about 1,300 mg, about 1,200 mg, inclusive; about 1,200 mg to about 1,800 mg, about 1,700 mg, about 1,600 mg, about 1,500 mg, about 1,400 mg, or about 1,300 mg, inclusive; about 1,300 mg to about 1,800 mg, about 1,700 mg, about 1,600 mg, about 1,500 mg, or about 1,400 mg, inclusive; about 1,400 mg to about 1,800 mg, about 1,700 mg, about 1,600 mg, or about 1,500 mg, inclusive; about 1,500 mg to about 1,800 mg, about 1,700 mg, or about 1,600 mg, inclusive; about 1,600 mg to about 1,800 mg, or about 1,700 mg, inclusive; or about 1,700 mg to about 1,800 mg, inclusive, of riboflavin is administered to the subject in need thereof (e.g., any of the subjects described herein). Any of the compositions provided herein can further include a colonic acidifier (e.g., a therapeutically effective amount of a colonic acidifier). Colonic acidifiers may be used to promote laxation in a subject (e.g., any of the subjects described herein). In some examples, any of the composition provided herein may include a colonic acidifier such as, e.g., lactulose. Lactulose is a synthetic disaccharide (4-O-β-D-galactopyranosil-D-fructofuranose). Lactulose can be produced, in small scale, by heat-treating milk. It may also be obtained from the conversion of lactose using an alkaline isomerization process. Lactulose may be obtained or manufactured as described in U.S. Pat. Nos. 3,546,206 and 5,071,530. Lactulose may also be used to decrease an amount or level of ammonia in a subject (e.g., the blood of a subject).

A composition provided herein can contain any appropriate amount of lactulose. In some examples of any of the compositions provided herein, about 0.1% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, about 1% w/w, or about 0.5% w/w, inclusive; about 0.2% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, about 1% w/w, or about 0.5% w/w, inclusive; about 0.5% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, or about 1% w/w, inclusive; about 1% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, or about 2% w/w, inclusive; about 2% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, or about 3% w/w, inclusive; about 3% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, or about 4% w/w, inclusive; about 4% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, or about 5% w/w inclusive; about 5% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, or about 6% w/w, inclusive; about 6% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, or about 8% w/w, inclusive; about 8% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, or about 10% w/w, inclusive; about 10% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, or about 12% w/w, inclusive; about 12% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, or about 14% w/w, inclusive; about 14% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, or about 16% w/w, inclusive; about 16% to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, or about 18% w/w, inclusive; about 18% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, or about 20% w/w, inclusive; about 20% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, or about 22% w/w, inclusive; about 22% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, or about 24% w/w, inclusive; about 24% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, or about 26% w/w, inclusive; about 26% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, or about 28% w/w, inclusive; about 28% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, or about 30% w/w, inclusive; about 30% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, or about 32% w/w, inclusive; about 32% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, inclusive; about 34% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, or about 36% w/w, inclusive; about 36% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, or about 38% w/w, inclusive; about 38% w/w to about 60% w/w, about 58% w/w, about 56% w/w, about 54% w/w, about 52% w/w, about 50% w/w, about 48% w/w, about 46% w/w, about 44% w/w, about 42% w/w, or about 40% w/w, inclusive; about 40% w/w to about 60% w/w, about 58% w/w, about 56% w/w, about 54% w/w, about 52% w/w, about 50% w/w, about 48% w/w, about 46% w/w, about 44% w/w, or about 42% w/w, inclusive; about 42% w/w to about 60% w/w, about 58% w/w, about 56% w/w, about 54% w/w, about 52% w/w, about 50% w/w, about 48% w/w, about 46% w/w, or about 44% w/w, inclusive; about 44% w/w to about 60% w/w, about 58% w/w, about 56% w/w, about 54% w/w, about 52% w/w, about 50% w/w, about 48% w/w, or about 46% w/w, inclusive; about 46% w/w to about 60% w/w, about 58% w/w, about 56% w/w, about 54% w/w, about 52% w/w, about 50% w/w, or about 48% w/w, inclusive; about 48% w/w to about 60% w/w, about 58% w/w, about 56% w/w, about 54% w/w, about 52% w/w, or about 50% w/w, inclusive; about 50% w/w to about 60% w/w, about 58% w/w, about 56% w/w, about 54% w/w, or about 52% w/w, inclusive; about 52% w/w to about 60% w/w, about 58% w/w, about 56% w/w, or about 54% w/w, inclusive; about 54% w/w to about 60% w/w, about 58% w/w, or about 56% w/w, inclusive; about 56% w/w to about 60% w/w, or about 58% w/w, inclusive; or about 58% w/w to about 60% w/w, inclusive, of the composition is lactulose.

In some embodiments, no more than 0.5% w/w, no more than 1% w/w, no more than 2% w/w, no more than 4% w/w, no more than 5% w/w, no more than 6% w/w, no more than 8% w/w, no more than 10% w/w, no more than 12% w/w, no more than 14% w/w, no more than 15% w/w, no more than 16% w/w, no more than 18% w/w, no more than 20% w/w, no more than 22% w/w, no more than 24% w/w, no more than 25% w/w, no more than 26% w/w, no more than 28% w/w, no more than 30% w/w, no more than 32% w/w, no more than 34% w/w, no more than 35% w/w, no more than 36% w/w, no more than 38% w/w, no more than 40% w/w, no more than 42% w/w, no more than 44% w/w, no more than 45% w/w, no more than 46% w/w, no more than 48% w/w, no more than 50% w/w, no more than 52% w/w, no more than 54% w/w, no more than 55% w/w, no more than 56% w/w, no more than 58% w/w, or no more than 60% w/w of any of the compositions described herein is lactulose.

In some examples, a single dose of any of the compositions provided herein can include from about 0.1 g to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, about 1 mg, or about 0.5 mg, inclusive; about 0.5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg, inclusive; about 1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, or about 5 mg, inclusive; about 5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, or about 10 mg, inclusive; about 10 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, or about 20 mg, inclusive; about 20 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, or about 40 mg, inclusive; about 40 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, or about 60 mg, inclusive; about 60 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, or about 80 mg, inclusive; about 80 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, or about 100 mg, inclusive; about 100 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, or about 150 mg, inclusive; about 150 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, or about 200 mg, inclusive; about 200 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, or about 250 mg, inclusive; about 250 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, or about 300 mg, inclusive; about 300 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, or about 350 mg, inclusive; about 350 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, or about 400 mg, inclusive; about 400 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, or about 450 mg, inclusive; about 450 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, or about 500 mg, inclusive; about 500 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, or about 600 mg, inclusive; about 600 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, or about 700 mg, inclusive; about 700 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, or about 800 mg, inclusive; about 800 mg to about 1,800 mg, about 1,700 mg, about 1,600 mg, about 1,500 mg, about 1,400 mg, about 1,300 mg, about 1,200 mg, about 1,100 mg, about 1,000 mg, or about 900 mg, inclusive; about 900 mg to about 1,800 mg, about 1,700 mg, about 1,600 mg, about 1,500 mg, about 1,400 mg, about 1,300 mg, about 1,200 mg, about 1,100 mg, or about 1,000 mg, inclusive; about 1,000 mg to about 1,800 mg, about 1,700 mg, about 1,600 mg, about 1,500 mg, about 1,400 mg, about 1,300 mg, about 1,200 mg, or about 1,100 mg, inclusive; about 1,100 mg to about 1,800 mg, about 1,700 mg, about 1,600 mg, about 1,500 mg, about 1,400 mg, about 1,300 mg, about 1,200 mg, inclusive; about 1,200 mg to about 1,800 mg, about 1,700 mg, about 1,600 mg, about 1,500 mg, about 1,400 mg, or about 1,300 mg, inclusive; about 1,300 mg to about 1,800 mg, about 1,700 mg, about 1,600 mg, about 1,500 mg, or about 1,400 mg, inclusive; about 1,400 mg to about 1,800 mg, about 1,700 mg, about 1,600 mg, or about 1,500 mg, inclusive; about 1,500 mg to about 1,800 mg, about 1,700 mg, or about 1,600 mg, inclusive; about 1,600 mg to about 1,800 mg, or about 1,700 mg, inclusive; or about 1,700 mg to about 1,800 mg, inclusive, of lactulose.

In some examples, a composition provided herein can be formulated to contain an amount of lactulose such that a daily dose of about 0.1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, about 1 mg, or about 0.5 mg, inclusive; about 0.5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg, inclusive; about 1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, or about 5 mg, inclusive; about 5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, or about 10 mg, inclusive; about 10 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, or about 20 mg, inclusive; about 20 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, or about 40 mg, inclusive; about 40 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, or about 60 mg, inclusive; about 60 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, or about 80 mg, inclusive; about 80 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, or about 100 mg, inclusive; about 100 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, or about 150 mg, inclusive; about 150 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, or about 200 mg, inclusive; about 200 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, or about 250 mg, inclusive; about 250 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, or about 300 mg, inclusive; about 300 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, or about 350 mg, inclusive; about 350 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, or about 400 mg, inclusive; about 400 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, or about 450 mg, inclusive; about 450 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, or about 500 mg, inclusive; about 500 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, or about 600 mg, inclusive; about 600 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, or about 700 mg, inclusive; about 700 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, or about 800 mg, inclusive; about 800 mg to about 1,800 mg, about 1,700 mg, about 1,600 mg, about 1,500 mg, about 1,400 mg, about 1,300 mg, about 1,200 mg, about 1,100 mg, about 1,000 mg, or about 900 mg, inclusive; about 900 mg to about 1,800 mg, about 1,700 mg, about 1,600 mg, about 1,500 mg, about 1,400 mg, about 1,300 mg, about 1,200 mg, about 1,100 mg, or about 1,000 mg, inclusive; about 1,000 mg to about 1,800 mg, about 1,700 mg, about 1,600 mg, about 1,500 mg, about 1,400 mg, about 1,300 mg, about 1,200 mg, or about 1,100 mg, inclusive; about 1,100 mg to about 1,800 mg, about 1,700 mg, about 1,600 mg, about 1,500 mg, about 1,400 mg, about 1,300 mg, about 1,200 mg, inclusive; about 1,200 mg to about 1,800 mg, about 1,700 mg, about 1,600 mg, about 1,500 mg, about 1,400 mg, or about 1,300 mg, inclusive; about 1,300 mg to about 1,800 mg, about 1,700 mg, about 1,600 mg, about 1,500 mg, or about 1,400 mg, inclusive; about 1,400 mg to about 1,800 mg, about 1,700 mg, about 1,600 mg, or about 1,500 mg, inclusive; about 1,500 mg to about 1,800 mg, about 1,700 mg, or about 1,600 mg, inclusive; about 1,600 mg to about 1,800 mg, or about 1,700 mg, inclusive; or about 1,700 mg to about 1,800 mg, inclusive, of lactulose is administered to the subject in need thereof (e.g., any of the subjects described herein).

In some examples, a composition provided herein can further include an ammonia reducer. In some cases, the ammonia reducer can include one or more antibiotics or combinations of antibiotics in a treatment against bacterial bowel infections. Suitable antibiotics may include, e.g., neomycin, metronidazole, rifaximin, or a combination thereof.

In some examples, any of the compositions provided herein can include one or more of neomycin, metronidazole, and rifaximin. Any of the compositions provided herein can include any appropriate amount of one or more of neomycin, metronidazole, and rifaximin. For example, about 0.1% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, about 1% w/w, or about 0.5% w/w, inclusive; about 0.2% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, about 1% w/w, or about 0.5% w/w, inclusive; about 0.5% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, or about 1% w/w, inclusive; about 1% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, or about 2% w/w, inclusive; about 2% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, or about 3% w/w, inclusive; about 3% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, or about 4% w/w, inclusive; about 4% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, or about 5% w/w inclusive; about 5% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, or about 6% w/w, inclusive; about 6% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, or about 8% w/w, inclusive; about 8% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, or about 10% w/w, inclusive; about 10% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, or about 12% w/w, inclusive; about 12% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, or about 14% w/w, inclusive; about 14% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, or about 16% w/w, inclusive; about 16% to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, or about 18% w/w, inclusive; about 18% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, or about 20% w/w, inclusive; about 20% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, or about 22% w/w, inclusive; about 22% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, or about 24% w/w, inclusive; about 24% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, or about 26% w/w, inclusive; about 26% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, or about 28% w/w, inclusive; about 28% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, or about 30% w/w, inclusive; about 30% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, or about 32% w/w, inclusive; about 32% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, inclusive; about 34% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, or about 36% w/w, inclusive; about 36% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, or about 38% w/w, inclusive; about 38% w/w to about 60% w/w, about 58% w/w, about 56% w/w, about 54% w/w, about 52% w/w, about 50% w/w, about 48% w/w, about 46% w/w, about 44% w/w, about 42% w/w, or about 40% w/w, inclusive; about 40% w/w to about 60% w/w, about 58% w/w, about 56% w/w, about 54% w/w, about 52% w/w, about 50% w/w, about 48% w/w, about 46% w/w, about 44% w/w, or about 42% w/w, inclusive; about 42% w/w to about 60% w/w, about 58% w/w, about 56% w/w, about 54% w/w, about 52% w/w, about 50% w/w, about 48% w/w, about 46% w/w, or about 44% w/w, inclusive; about 44% w/w to about 60% w/w, about 58% w/w, about 56% w/w, about 54% w/w, about 52% w/w, about 50% w/w, about 48% w/w, or about 46% w/w, inclusive; about 46% w/w to about 60% w/w, about 58% w/w, about 56% w/w, about 54% w/w, about 52% w/w, about 50% w/w, or about 48% w/w, inclusive; about 48% w/w to about 60% w/w, about 58% w/w, about 56% w/w, about 54% w/w, about 52% w/w, or about 50% w/w, inclusive; about 50% w/w to about 60% w/w, about 58% w/w, about 56% w/w, about 54% w/w, or about 52% w/w, inclusive; about 52% w/w to about 60% w/w, about 58% w/w, about 56% w/w, or about 54% w/w, inclusive; about 54% w/w to about 60% w/w, about 58% w/w, or about 56% w/w, inclusive; about 56% w/w to about 60% w/w, or about 58% w/w, inclusive; or about 58% w/w to about 60% w/w, inclusive, of any of the compositions provided herein is neomycin, metronidazole, or rifaximin, or the sum of two or more of neomycin, metronidazole, and rifaximin.

In some examples, no more than 1% w/w, no more than about 2% w/w, no more than 4% w/w, no more than 5% w/w, no more than 6% w/w, no more than 8% w/w, no more than 10% w/w, no more than 12% w/w, no more than 14% w/w, no more than 15% w/w, no more than 16% w/w, no more than 18% w/w, no more than 20% w/w, no more than about 22% w/w, no more than 24% w/w, no more than 25% w/w, no more than 26% w/w, no more than 28% w/w, no more than 30% w/w, no more than 32% w/w, no more than 34% w/w, no more than 35% w/w, no more than 36% w/w, no more than 38% w/w, no more than 40% w/w, no more than 42% w/w, no more than 44% w/w, no more than 45% w/w, no more than 46% w/w, no more than 48% w/w, no more than 50% w/w, no more than 52% w/w, no more than 54% w/w, no more than 56% w/w, no more than 58% w/w, or no more than 60% w/w, of any of the compositions provided herein is neomycin, metronidazole, or rifaximin, or the sum of two or more of neomycin, metronidazole, and rifaximin.

In some examples, a single dose of any of the compositions provided herein can include from about 0.1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, about 1 mg, or about 0.5 mg, inclusive; about 0.5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg, inclusive; about 1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, or about 5 mg, inclusive; about 5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, or about 10 mg, inclusive; about 10 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, or about 20 mg, inclusive; about 20 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, or about 40 mg, inclusive; about 40 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, or about 60 mg, inclusive; about 60 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, or about 80 mg, inclusive; about 80 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, or about 100 mg, inclusive; about 100 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, or about 150 mg, inclusive; about 150 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, or about 200 mg, inclusive; about 200 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, or about 250 mg, inclusive; about 250 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, or about 300 mg, inclusive; about 300 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, or about 350 mg, inclusive; about 350 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, or about 400 mg, inclusive; about 400 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, or about 450 mg, inclusive; about 450 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, or about 500 mg, inclusive; about 500 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, or about 600 mg, inclusive; about 600 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, or about 700 mg, inclusive; about 700 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, or about 800 mg, inclusive; about 800 mg to about 1,800 mg, about 1,700 mg, about 1,600 mg, about 1,500 mg, about 1,400 mg, about 1,300 mg, about 1,200 mg, about 1,100 mg, about 1,000 mg, or about 900 mg, inclusive; about 900 mg to about 1,800 mg, about 1,700 mg, about 1,600 mg, about 1,500 mg, about 1,400 mg, about 1,300 mg, about 1,200 mg, about 1,100 mg, or about 1,000 mg, inclusive; about 1,000 mg to about 1,800 mg, about 1,700 mg, about 1,600 mg, about 1,500 mg, about 1,400 mg, about 1,300 mg, about 1,200 mg, or about 1,100 mg, inclusive; about 1,100 mg to about 1,800 mg, about 1,700 mg, about 1,600 mg, about 1,500 mg, about 1,400 mg, about 1,300 mg, about 1,200 mg, inclusive; about 1,200 mg to about 1,800 mg, about 1,700 mg, about 1,600 mg, about 1,500 mg, about 1,400 mg, or about 1,300 mg, inclusive; about 1,300 mg to about 1,800 mg, about 1,700 mg, about 1,600 mg, about 1,500 mg, or about 1,400 mg, inclusive; about 1,400 mg to about 1,800 mg, about 1,700 mg, about 1,600 mg, or about 1,500 mg, inclusive; about 1,500 mg to about 1,800 mg, about 1,700 mg, or about 1,600 mg, inclusive; about 1,600 mg to about 1,800 mg, or about 1,700 mg, inclusive; or about 1,700 mg to about 1,800 mg, inclusive, of neomycin, metronidazole, or rifaximin, or a mixture of two or more of neomycin, metronidazole, and rifaximin.

In some examples, any of the compositions provided herein can include an amount (e.g., a therapeutically effective amount) of an ammonia reducer, e.g., one or more of neomycin, metronidazole, and rifaximin, such that a daily dose of about 0.1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, about 1 mg, or about 0.5 mg, inclusive; about 0.5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg, inclusive; about 1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, or about 5 mg, inclusive; about 5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, or about 10 mg, inclusive; about 10 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, or about 20 mg, inclusive; about 20 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, or about 40 mg, inclusive; about 40 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, or about 60 mg, inclusive; about 60 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, or about 80 mg, inclusive; about 80 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, or about 100 mg, inclusive; about 100 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, or about 150 mg, inclusive; about 150 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, or about 200 mg, inclusive; about 200 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, or about 250 mg, inclusive; about 250 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, or about 300 mg, inclusive; about 300 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, or about 350 mg, inclusive; about 350 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, or about 400 mg, inclusive; about 400 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, or about 450 mg, inclusive; about 450 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, or about 500 mg, inclusive; about 500 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, or about 600 mg, inclusive; about 600 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, or about 700 mg, inclusive; about 700 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, or about 800 mg, inclusive; about 800 mg to about 1,800 mg, about 1,700 mg, about 1,600 mg, about 1,500 mg, about 1,400 mg, about 1,300 mg, about 1,200 mg, about 1,100 mg, about 1,000 mg, or about 900 mg, inclusive; about 900 mg to about 1,800 mg, about 1,700 mg, about 1,600 mg, about 1,500 mg, about 1,400 mg, about 1,300 mg, about 1,200 mg, about 1,100 mg, or about 1,000 mg, inclusive; about 1,000 mg to about 1,800 mg, about 1,700 mg, about 1,600 mg, about 1,500 mg, about 1,400 mg, about 1,300 mg, about 1,200 mg, or about 1,100 mg, inclusive; about 1,100 mg to about 1,800 mg, about 1,700 mg, about 1,600 mg, about 1,500 mg, about 1,400 mg, about 1,300 mg, about 1,200 mg, inclusive; about 1,200 mg to about 1,800 mg, about 1,700 mg, about 1,600 mg, about 1,500 mg, about 1,400 mg, or about 1,300 mg, inclusive; about 1,300 mg to about 1,800 mg, about 1,700 mg, about 1,600 mg, about 1,500 mg, or about 1,400 mg, inclusive; about 1,400 mg to about 1,800 mg, about 1,700 mg, about 1,600 mg, or about 1,500 mg, inclusive; about 1,500 mg to about 1,800 mg, about 1,700 mg, or about 1,600 mg, inclusive; about 1,600 mg to about 1,800 mg, or about 1,700 mg, inclusive; or about 1,700 mg to about 1,800 mg, inclusive, of an ammonia reducer, or the sum of two or more ammonia reducers, is administered to the subject in need thereof (e.g., any of the subjects described herein).

Any of the compositions provided herein can further include an ammonia sequestering agent. In some cases, the composition can include one or more ammonia sequestering agents. Exemplary ammonia sequestering agents include L-Ornithine L-aspartate, L-ornithine-α-ketoglutarate, or L-ornithine-phenylacetate, or a combination thereof.

In some examples, a composition can contain any appropriate amount (e.g., a therapeutically effective amount) of an ammonia sequestering agent. A composition provided herein can contain any appropriate amount of one or more of L-Ornithine L-aspartate, L-ornithine-α-ketoglutarate, and L-ornithine-phenylacetate. For example, about 0.1% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, about 1% w/w, or about 0.5% w/w, inclusive; about 0.2% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, about 1% w/w, or about 0.5% w/w, inclusive; about 0.5% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, or about 1% w/w, inclusive; about 1% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, or about 2% w/w, inclusive; about 2% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, or about 3% w/w, inclusive; about 3% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, or about 4% w/w, inclusive; about 4% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, or about 5% w/w inclusive; about 5% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, or about 6% w/w, inclusive; about 6% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, or about 8% w/w, inclusive; about 8% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, or about 10% w/w, inclusive; about 10% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, or about 12% w/w, inclusive; about 12% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, or about 14% w/w, inclusive; about 14% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, or about 16% w/w, inclusive; about 16% to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, or about 18% w/w, inclusive; about 18% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, or about 20% w/w, inclusive; about 20% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, or about 22% w/w, inclusive; about 22% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, or about 24% w/w, inclusive; about 24% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, or about 26% w/w, inclusive; about 26% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, or about 28% w/w, inclusive; about 28% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, or about 30% w/w, inclusive; about 30% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, or about 32% w/w, inclusive; about 32% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, inclusive; about 34% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, or about 36% w/w, inclusive; about 36% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, or about 38% w/w, inclusive; about 38% w/w to about 60% w/w, about 58% w/w, about 56% w/w, about 54% w/w, about 52% w/w, about 50% w/w, about 48% w/w, about 46% w/w, about 44% w/w, about 42% w/w, or about 40% w/w, inclusive; about 40% w/w to about 60% w/w, about 58% w/w, about 56% w/w, about 54% w/w, about 52% w/w, about 50% w/w, about 48% w/w, about 46% w/w, about 44% w/w, or about 42% w/w, inclusive; about 42% w/w to about 60% w/w, about 58% w/w, about 56% w/w, about 54% w/w, about 52% w/w, about 50% w/w, about 48% w/w, about 46% w/w, or about 44% w/w, inclusive; about 44% w/w to about 60% w/w, about 58% w/w, about 56% w/w, about 54% w/w, about 52% w/w, about 50% w/w, about 48% w/w, or about 46% w/w, inclusive; about 46% w/w to about 60% w/w, about 58% w/w, about 56% w/w, about 54% w/w, about 52% w/w, about 50% w/w, or about 48% w/w, inclusive; about 48% w/w to about 60% w/w, about 58% w/w, about 56% w/w, about 54% w/w, about 52% w/w, or about 50% w/w, inclusive; about 50% w/w to about 60% w/w, about 58% w/w, about 56% w/w, about 54% w/w, or about 52% w/w, inclusive; about 52% w/w to about 60% w/w, about 58% w/w, about 56% w/w, or about 54% w/w, inclusive; about 54% w/w to about 60% w/w, about 58% w/w, or about 56% w/w, inclusive; about 56% w/w to about 60% w/w, or about 58% w/w, inclusive; or about 58% w/w to about 60% w/w, inclusive, of any of the compositions provided herein is L-Ornithine L-aspartate, L-ornithine-α-ketoglutarate, or L-ornithine-phenylacetate, or the sum of two or more of L-ornithine L-aspartate, L-ornithine-α-ketoglutarate, or L-ornithine-phenylacetate.

In some examples, no more than 1% w/w, no more than about 2% w/w, no more than 4% w/w, no more than 5% w/w, no more than 6% w/w, no more than 8% w/w, no more than 10% w/w, no more than 12% w/w, no more than 14% w/w, no more than 15% w/w, no more than 16% w/w, no more than 18% w/w, no more than 20% w/w, no more than about 22% w/w, no more than 24% w/w, no more than 25% w/w, no more than 26% w/w, no more than 28% w/w, no more than 30% w/w, no more than 32% w/w, no more than 34% w/w, no more than 35% w/w, no more than 36% w/w, no more than 38% w/w, no more than 40% w/w, no more than 42% w/w, no more than 44% w/w, no more than 45% w/w, no more than 46% w/w, no more than 48% w/w, no more than 50% w/w, no more than 52% w/w, no more than 54% w/w, no more than 56% w/w, no more than 58% w/w, or no more than 60% w/w, of any of the compositions provided herein is L-ornithine L-aspartate, L-ornithine-α-ketoglutarate, or L-ornithine-phenylacetate, or the sum of two or more of L-ornithine L-aspartate, L-ornithine-α-ketoglutarate, or L-ornithine-phenylacetate.

In some examples, a single dose of any of the compositions provided herein can include from about 0.1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, about 1 mg, or about 0.5 mg, inclusive; about 0.5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg, inclusive; about 1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, or about 5 mg, inclusive; about 5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, or about 10 mg, inclusive; about 10 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, or about 20 mg, inclusive; about 20 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, or about 40 mg, inclusive; about 40 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, or about 60 mg, inclusive; about 60 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, or about 80 mg, inclusive; about 80 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, or about 100 mg, inclusive; about 100 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, or about 150 mg, inclusive; about 150 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, or about 200 mg, inclusive; about 200 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, or about 250 mg, inclusive; about 250 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, or about 300 mg, inclusive; about 300 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, or about 350 mg, inclusive; about 350 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, or about 400 mg, inclusive; about 400 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, or about 450 mg, inclusive; about 450 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, or about 500 mg, inclusive; about 500 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, or about 600 mg, inclusive; about 600 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, or about 700 mg, inclusive; about 700 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, or about 800 mg, inclusive; about 800 mg to about 1,800 mg, about 1,700 mg, about 1,600 mg, about 1,500 mg, about 1,400 mg, about 1,300 mg, about 1,200 mg, about 1,100 mg, about 1,000 mg, or about 900 mg, inclusive; about 900 mg to about 1,800 mg, about 1,700 mg, about 1,600 mg, about 1,500 mg, about 1,400 mg, about 1,300 mg, about 1,200 mg, about 1,100 mg, or about 1,000 mg, inclusive; about 1,000 mg to about 1,800 mg, about 1,700 mg, about 1,600 mg, about 1,500 mg, about 1,400 mg, about 1,300 mg, about 1,200 mg, or about 1,100 mg, inclusive; about 1,100 mg to about 1,800 mg, about 1,700 mg, about 1,600 mg, about 1,500 mg, about 1,400 mg, about 1,300 mg, about 1,200 mg, inclusive; about 1,200 mg to about 1,800 mg, about 1,700 mg, about 1,600 mg, about 1,500 mg, about 1,400 mg, or about 1,300 mg, inclusive; about 1,300 mg to about 1,800 mg, about 1,700 mg, about 1,600 mg, about 1,500 mg, or about 1,400 mg, inclusive; about 1,400 mg to about 1,800 mg, about 1,700 mg, about 1,600 mg, or about 1,500 mg, inclusive; about 1,500 mg to about 1,800 mg, about 1,700 mg, or about 1,600 mg, inclusive; about 1,600 mg to about 1,800 mg, or about 1,700 mg, inclusive; or about 1,700 mg to about 1,800 mg, inclusive, of L-Ornithine L-aspartate, L-ornithine-α-ketoglutarate, or L-ornithine-phenylacetate, or the sum of two or more of L-ornithine L-aspartate, L-ornithine-α-ketoglutarate, and L-ornithine-phenylacetate.

In some examples, a composition provided herein can be formulated to contain an amount of one or more of L-Ornithine L-aspartate, L-ornithine-α-ketoglutarate, and L-ornithine-phenylacetate, such that a daily dose of about 0.1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, about 1 mg, or about 0.5 mg, inclusive; about 0.5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg, inclusive; about 1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, or about 5 mg, inclusive; about 5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, or about 10 mg, inclusive; about 10 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, or about 20 mg, inclusive; about 20 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, or about 40 mg, inclusive; about 40 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, or about 60 mg, inclusive; about 60 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, or about 80 mg, inclusive; about 80 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, or about 100 mg, inclusive; about 100 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, or about 150 mg, inclusive; about 150 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, or about 200 mg, inclusive; about 200 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, or about 250 mg, inclusive; about 250 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, or about 300 mg, inclusive; about 300 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, or about 350 mg, inclusive; about 350 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, or about 400 mg, inclusive; about 400 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, or about 450 mg, inclusive; about 450 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, or about 500 mg, inclusive; about 500 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, or about 600 mg, inclusive; about 600 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, or about 700 mg, inclusive; about 700 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, or about 800 mg, inclusive; about 800 mg to about 1,800 mg, about 1,700 mg, about 1,600 mg, about 1,500 mg, about 1,400 mg, about 1,300 mg, about 1,200 mg, about 1,100 mg, about 1,000 mg, or about 900 mg, inclusive; about 900 mg to about 1,800 mg, about 1,700 mg, about 1,600 mg, about 1,500 mg, about 1,400 mg, about 1,300 mg, about 1,200 mg, about 1,100 mg, or about 1,000 mg, inclusive; about 1,000 mg to about 1,800 mg, about 1,700 mg, about 1,600 mg, about 1,500 mg, about 1,400 mg, about 1,300 mg, about 1,200 mg, or about 1,100 mg, inclusive; about 1,100 mg to about 1,800 mg, about 1,700 mg, about 1,600 mg, about 1,500 mg, about 1,400 mg, about 1,300 mg, about 1,200 mg, inclusive; about 1,200 mg to about 1,800 mg, about 1,700 mg, about 1,600 mg, about 1,500 mg, about 1,400 mg, or about 1,300 mg, inclusive; about 1,300 mg to about 1,800 mg, about 1,700 mg, about 1,600 mg, about 1,500 mg, or about 1,400 mg, inclusive; about 1,400 mg to about 1,800 mg, about 1,700 mg, about 1,600 mg, or about 1,500 mg, inclusive; about 1,500 mg to about 1,800 mg, about 1,700 mg, or about 1,600 mg, inclusive; about 1,600 mg to about 1,800 mg, or about 1,700 mg, inclusive; or about 1,700 mg to about 1,800 mg, inclusive, of L-Ornithine L-aspartate, L-ornithine-α-ketoglutarate, or L-ornithine-phenylacetate, or the sum of two or more of L-ornithine L-aspartate, L-ornithine-α-ketoglutarate, and L-ornithine-phenylacetate is administered to a subject (e.g., any of the subjects described herein).

Any of the compositions provided herein can further include an intestinal deodorizer. In some cases, the composition can include one or more intestinal deodorizers. Suitable intestinal deodorizers may include one or both of charcoal and copper chlorophyllin.

In some examples, any of the compositions provided herein can include one or more of intestinal deodorizers. Any of the compositions provided herein can include any appropriate amount of an intestinal deodorizer such as, e.g., one or both of charcoal and copper chlorophyllin. For example, about 0.1% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, about 1% w/w, or about 0.5% w/w, inclusive; about 0.2% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, about 1% w/w, or about 0.5% w/w, inclusive; about 0.5% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, or about 1% w/w, inclusive; about 1% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, or about 2% w/w, inclusive; about 2% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, or about 3% w/w, inclusive; about 3% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, or about 4% w/w, inclusive; about 4% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, or about 5% w/w inclusive; about 5% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, or about 6% w/w, inclusive; about 6% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, or about 8% w/w, inclusive; about 8% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, or about 10% w/w, inclusive; about 10% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, or about 12% w/w, inclusive; about 12% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, or about 14% w/w, inclusive; about 14% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, or about 16% w/w, inclusive; about 16% to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, or about 18% w/w, inclusive; about 18% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, or about 20% w/w, inclusive; about 20% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, or about 22% w/w, inclusive; about 22% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, or about 24% w/w, inclusive; about 24% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, or about 26% w/w, inclusive; about 26% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, or about 28% w/w, inclusive; about 28% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, or about 30% w/w, inclusive; about 30% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, or about 32% w/w, inclusive; about 32% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, inclusive; about 34% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, or about 36% w/w, inclusive; about 36% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, or about 38% w/w, inclusive; about 38% w/w to about 60% w/w, about 58% w/w, about 56% w/w, about 54% w/w, about 52% w/w, about 50% w/w, about 48% w/w, about 46% w/w, about 44% w/w, about 42% w/w, or about 40% w/w, inclusive; about 40% w/w to about 60% w/w, about 58% w/w, about 56% w/w, about 54% w/w, about 52% w/w, about 50% w/w, about 48% w/w, about 46% w/w, about 44% w/w, or about 42% w/w, inclusive; about 42% w/w to about 60% w/w, about 58% w/w, about 56% w/w, about 54% w/w, about 52% w/w, about 50% w/w, about 48% w/w, about 46% w/w, or about 44% w/w, inclusive; about 44% w/w to about 60% w/w, about 58% w/w, about 56% w/w, about 54% w/w, about 52% w/w, about 50% w/w, about 48% w/w, or about 46% w/w, inclusive; about 46% w/w to about 60% w/w, about 58% w/w, about 56% w/w, about 54% w/w, about 52% w/w, about 50% w/w, or about 48% w/w, inclusive; about 48% w/w to about 60% w/w, about 58% w/w, about 56% w/w, about 54% w/w, about 52% w/w, or about 50% w/w, inclusive; about 50% w/w to about 60% w/w, about 58% w/w, about 56% w/w, about 54% w/w, or about 52% w/w, inclusive; about 52% w/w to about 60% w/w, about 58% w/w, about 56% w/w, or about 54% w/w, inclusive; about 54% w/w to about 60% w/w, about 58% w/w, or about 56% w/w, inclusive; about 56% w/w to about 60% w/w, or about 58% w/w, inclusive; or about 58% w/w to about 60% w/w, inclusive, of any of the compositions provided herein is an intestinal deodorizer, e.g., charcoal or copper chlorophyllin, or the sum of charcoal and copper chlorophyllin.

In some examples, no more than 1% w/w, no more than about 2% w/w, no more than 4% w/w, no more than 5% w/w, no more than 6% w/w, no more than 8% w/w, no more than 10% w/w, no more than 12% w/w, no more than 14% w/w, no more than 15% w/w, no more than 16% w/w, no more than 18% w/w, no more than 20% w/w, no more than about 22% w/w, no more than 24% w/w, no more than 25% w/w, no more than 26% w/w, no more than 28% w/w, no more than 30% w/w, no more than 32% w/w, no more than 34% w/w, no more than 35% w/w, no more than 36% w/w, no more than 38% w/w, no more than 40% w/w, no more than 42% w/w, no more than 44% w/w, no more than 45% w/w, no more than 46% w/w, no more than 48% w/w, no more than 50% w/w, no more than 52% w/w, no more than 54% w/w, no more than 56% w/w, no more than 58% w/w, or no more than 60% w/w, of any of the compositions provided herein is an intestinal deodorizer, e.g., charcoal or copper chlorophyllin, or the sum of charcoal and copper chlorophyllin.

In some examples, a single dose of any of the compositions provided herein can include from about 0.1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, about 1 mg, or about 0.5 mg, inclusive; about 0.5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg, inclusive; about 1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, or about 5 mg, inclusive; about 5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, or about 10 mg, inclusive; about 10 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, or about 20 mg, inclusive; about 20 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, or about 40 mg, inclusive; about 40 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, or about 60 mg, inclusive; about 60 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, or about 80 mg, inclusive; about 80 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, or about 100 mg, inclusive; about 100 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, or about 150 mg, inclusive; about 150 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, or about 200 mg, inclusive; about 200 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, or about 250 mg, inclusive; about 250 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, or about 300 mg, inclusive; about 300 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, or about 350 mg, inclusive; about 350 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, or about 400 mg, inclusive; about 400 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, or about 450 mg, inclusive; about 450 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, or about 500 mg, inclusive; about 500 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, or about 600 mg, inclusive; about 600 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, or about 700 mg, inclusive; about 700 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, or about 800 mg, inclusive; about 800 mg to about 1,800 mg, about 1,700 mg, about 1,600 mg, about 1,500 mg, about 1,400 mg, about 1,300 mg, about 1,200 mg, about 1,100 mg, about 1,000 mg, or about 900 mg, inclusive; about 900 mg to about 1,800 mg, about 1,700 mg, about 1,600 mg, about 1,500 mg, about 1,400 mg, about 1,300 mg, about 1,200 mg, about 1,100 mg, or about 1,000 mg, inclusive; about 1,000 mg to about 1,800 mg, about 1,700 mg, about 1,600 mg, about 1,500 mg, about 1,400 mg, about 1,300 mg, about 1,200 mg, or about 1,100 mg, inclusive; about 1,100 mg to about 1,800 mg, about 1,700 mg, about 1,600 mg, about 1,500 mg, about 1,400 mg, about 1,300 mg, about 1,200 mg, inclusive; about 1,200 mg to about 1,800 mg, about 1,700 mg, about 1,600 mg, about 1,500 mg, about 1,400 mg, or about 1,300 mg, inclusive; about 1,300 mg to about 1,800 mg, about 1,700 mg, about 1,600 mg, about 1,500 mg, or about 1,400 mg, inclusive; about 1,400 mg to about 1,800 mg, about 1,700 mg, about 1,600 mg, or about 1,500 mg, inclusive; about 1,500 mg to about 1,800 mg, about 1,700 mg, or about 1,600 mg, inclusive; about 1,600 mg to about 1,800 mg, or about 1,700 mg, inclusive; or about 1,700 mg to about 1,800 mg, inclusive, of an intestinal deodorizer, e.g., charcoal or copper chlorophyllin, or the sum of charcoal and copper chlorophyllin.

In some examples, a composition provided herein can be formulated to include an amount of an intestinal deodorizer, e.g., one or both of charcoal and copper chlorophyllin, such that a daily dose of about 0.1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, about 1 mg, or about 0.5 mg, inclusive; about 0.5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg, inclusive; about 1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, or about 5 mg, inclusive; about 5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, or about 10 mg, inclusive; about 10 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, or about 20 mg, inclusive; about 20 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, or about 40 mg, inclusive; about 40 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, or about 60 mg, inclusive; about 60 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, or about 80 mg, inclusive; about 80 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, or about 100 mg, inclusive; about 100 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, or about 150 mg, inclusive; about 150 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, or about 200 mg, inclusive; about 200 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, or about 250 mg, inclusive; about 250 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, or about 300 mg, inclusive; about 300 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, or about 350 mg, inclusive; about 350 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, or about 400 mg, inclusive; about 400 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, or about 450 mg, inclusive; about 450 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, or about 500 mg, inclusive; about 500 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, or about 600 mg, inclusive; about 600 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, or about 700 mg, inclusive; about 700 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, or about 800 mg, inclusive; about 800 mg to about 1,800 mg, about 1,700 mg, about 1,600 mg, about 1,500 mg, about 1,400 mg, about 1,300 mg, about 1,200 mg, about 1,100 mg, about 1,000 mg, or about 900 mg, inclusive; about 900 mg to about 1,800 mg, about 1,700 mg, about 1,600 mg, about 1,500 mg, about 1,400 mg, about 1,300 mg, about 1,200 mg, about 1,100 mg, or about 1,000 mg, inclusive; about 1,000 mg to about 1,800 mg, about 1,700 mg, about 1,600 mg, about 1,500 mg, about 1,400 mg, about 1,300 mg, about 1,200 mg, or about 1,100 mg, inclusive; about 1,100 mg to about 1,800 mg, about 1,700 mg, about 1,600 mg, about 1,500 mg, about 1,400 mg, about 1,300 mg, about 1,200 mg, inclusive; about 1,200 mg to about 1,800 mg, about 1,700 mg, about 1,600 mg, about 1,500 mg, about 1,400 mg, or about 1,300 mg, inclusive; about 1,300 mg to about 1,800 mg, about 1,700 mg, about 1,600 mg, about 1,500 mg, or about 1,400 mg, inclusive; about 1,400 mg to about 1,800 mg, about 1,700 mg, about 1,600 mg, or about 1,500 mg, inclusive; about 1,500 mg to about 1,800 mg, about 1,700 mg, or about 1,600 mg, inclusive; about 1,600 mg to about 1,800 mg, or about 1,700 mg, inclusive; or about 1,700 mg to about 1,800 mg, inclusive, of charcoal or copper chlorophyllin, or the sum of charcoal and copper chlorophyllin is administered to a subject (e.g., any of the subjects described herein).

Any of the compositions provided herein can further include one or more probiotics. Examples of suitable probiotics can include Lactobacillus species, Bifidobacterium species, Enterococcus species, or Streptococcus thermophiles, or a combination thereof. Lactobacillus species can include, but are not limited to, L. acidophilus, L. paracasei, L. fermentum, L. rhamnosus, L. johnsonii, L. plantarum, L. reuteri, L. salivarius, L. brevis, L. bulgaricus, L. helveticus, L. grasseri, L. casei, or L. lactis, or a combination thereof. Examples of suitable Bifidobacterium species can include, but are not limited to, B. bifidum, B. breve, B. infantis, B. longum, or B. lactis, or a combination thereof. Examples of suitable Enterococcus species can include, but are not limited to, E. faceum or E. faecalis, or a combination thereof.

In some examples, any of the compositions provided herein can include any appropriate amount (e.g., a therapeutically effective amount) of a probiotic. A composition provided herein can include any appropriate amount of one or more of Lactobacillus species, Bifidobacterium species, Enterococcus species, and Streptococcus thermophiles. For example, about 0.1% w/w to about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, about 1% w/w, or about 0.5% w/w, inclusive; about 0.2% w/w to about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, about 1% w/w, or about 0.5% w/w, inclusive; about 0.5% w/w to about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, or about 1% w/w, inclusive; about 1% w/w to about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, or about 2% w/w, inclusive; about 2% w/w to about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, or about 3% w/w, inclusive; about 3% w/w to about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, or about 4% w/w, inclusive; about 4% w/w to about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, or about 5% w/w, inclusive; about 5% w/w to about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, or about 6% w/w, inclusive; about 6% w/w to about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, or about 8% w/w, inclusive; about 8% w/w to about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, or about 10% w/w, inclusive; about 10% w/w to about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, or about 12% w/w, inclusive; about 12% w/w to about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, or about 14% w/w, inclusive; about 14% w/w to about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, or about 16% w/w, inclusive; about 16% to about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, or about 18% w/w, inclusive; about 18% w/w to about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, or about 20% w/w, inclusive; about 20% w/w to about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, or about 22% w/w, inclusive; about 22% w/w to about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, or about 24% w/w, inclusive; about 24% w/w to about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, or about 26% w/w, inclusive; about 26% w/w to about 50% w/w, about 48% w/w, about 46% w/w, about 44% w/w, about 42% w/w, about 40% w/w, about 38% w/w, about 36% w/w, about 34% w/w, about 32% w/w, about 30% w/w, or about 28% w/w, inclusive; about 28% w/w to about 50% w/w, about 48% w/w, about 46% w/w, about 44% w/w, about 42% w/w, about 40% w/w, about 38% w/w, about 36% w/w, about 34% w/w, about 32% w/w, or about 30% w/w, inclusive; about 30% w/w to about 50% w/w, about 48% w/w, about 46% w/w, about 44% w/w, about 42% w/w, about 40% w/w, about 38% w/w, about 36% w/w, about 34% w/w, or about 32% w/w, inclusive; about 32% w/w to about 50% w/w, about 48% w/w, about 46% w/w, about 44% w/w, about 42% w/w, about 40% w/w, about 38% w/w, about 36% w/w, about 34% w/w, inclusive; about 34% w/w to about 50% w/w, about 48% w/w, about 46% w/w, about 44% w/w, about 42% w/w, about 40% w/w, about 38% w/w, or about 36% w/w, inclusive; about 36% w/w to about 50% w/w, about 48% w/w, about 46% w/w, about 44% w/w, about 42% w/w, about 40% w/w, or about 38% w/w, inclusive; about 38% w/w to about 50% w/w, about 48% w/w, about 46% w/w, about 44% w/w, about 42% w/w, or about 40% w/w, inclusive; about 40% w/w to about 50% w/w, about 48% w/w, about 46% w/w, about 44% w/w, or about 42% w/w, inclusive; about 42% w/w to about 50% w/w, about 48% w/w, about 46% w/w, or about 44% w/w, inclusive; about 44% w/w to about 50% w/w, about 48% w/w, or about 46% w/w, inclusive; about 46% w/w to about 50% w/w or about 48% w/w, inclusive; or about 48% w/w to about 50% w/w, of any of the compositions provided herein is any of one or more of Lactobacillus species, Bifidobacterium species, Enterococcus species, and Streptococcus thermophiles.

In some examples, no more than 1% w/w, no more than about 2% w/w, no more than 4% w/w, no more than 5% w/w, no more than 6% w/w, no more than 8% w/w, no more than 10% w/w, no more than 12% w/w, no more than 14% w/w, no more than 15% w/w, no more than 16% w/w, no more than 18% w/w, no more than 20% w/w, no more than about 22% w/w, no more than 24% w/w, no more than 25% w/w, no more than 26% w/w, no more than 28% w/w, no more than 30% w/w, no more than 32% w/w, no more than 34% w/w, no more than 35% w/w, no more than 36% w/w, no more than 38% w/w, no more than 40% w/w, no more than 42% w/w, no more than 44% w/w, no more than 45% w/w, no more than 46% w/w, no more than 48% w/w, or no more than 50% w/w, of any of the compositions provided herein is a Lactobacillus species, a Bifidobacterium species, a Enterococcus species, or a Streptococcus thermophile, or the sum of two or more of a Lactobacillus species, a Bifidobacterium species, a Enterococcus species, and a Streptococcus thermophile.

In some examples, a single dose of any of the compositions provided herein can include from about 0.1 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, about 1 mg, or about 0.5 mg, inclusive; about 0.5 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg, inclusive; about 1 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, or about 5 mg, inclusive; about 5 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, or about 10 mg, inclusive; about 10 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, or about 20 mg, inclusive; about 20 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, or about 40 mg, inclusive; about 40 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, or about 60 mg, inclusive; about 60 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, or about 80 mg, inclusive; about 80 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, or about 100 mg, inclusive; about 100 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, or about 150 mg, inclusive; about 150 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, or about 200 mg, inclusive; about 200 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, or about 250 mg, inclusive; about 250 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, or about 300 mg, inclusive; about 300 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, or about 350 mg, inclusive; about 350 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, or about 400 mg, inclusive; about 400 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, or about 450 mg, inclusive; about 450 mg to about 1,500 mg, about 1,400 mg, about 1,300 mg, about 1,200 mg, about 1,100 mg, about 1,000 mg, about 900 mg, about 800 mg, about 700 mg, about 600 mg, or about 500 mg, inclusive; about 500 mg to about 1,500 mg, about 1,400 mg, about 1,300 mg, about 1,200 mg, about 1,100 mg, about 1,000 mg, about 900 mg, about 800 mg, about 700 mg, or about 600 mg, inclusive; about 600 mg to about 1,500 mg, about 1,400 mg, about 1,300 mg, about 1,200 mg, about 1,100 mg, about 1,000 mg, about 900 mg, about 800 mg, or about 700 mg, inclusive; about 700 mg to about 1,500 mg, about 1,400 mg, about 1,300 mg, about 1,200 mg, about 1,100 mg, about 1,000 mg, about 900 mg, or about 800 mg, inclusive; about 800 mg to about 1,500 mg, about 1,400 mg, about 1,300 mg, about 1,200 mg, about 1,100 mg, about 1,000 mg, or about 900 mg, inclusive; about 900 mg to about 1,500 mg, about 1,400 mg, about 1,300 mg, about 1,200 mg, about 1,100 mg, or about 1,000 mg, inclusive; about 1,000 mg to about 1,500 mg, about 1,400 mg, about 1,300 mg, about 1,200 mg, or about 1,100 mg, inclusive; about 1,100 mg to about 1,500 mg, about 1,400 mg, about 1,300 mg, or about 1,200 mg, inclusive; about 1,200 mg to about 1,500 mg, about 1,400 mg, or about 1,300 mg, inclusive; about 1,300 mg to about 1,500 mg or about 1,400 mg, inclusive; or about 1,400 mg to about 1,500 mg, inclusive, of any of one or more of Lactobacillus species, Bifidobacterium species, Enterococcus species, and Streptococcus thermophiles.

In some examples, a composition provided herein can be formulated to include an amount of one or more probiotic such that a daily dose of about 0.1 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, about 1 mg, or about 0.5 mg, inclusive; about 0.5 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg, inclusive; about 1 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, or about 5 mg, inclusive; about 5 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, or about 10 mg, inclusive; about 10 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, or about 20 mg, inclusive; about 20 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, or about 40 mg, inclusive; about 40 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, or about 60 mg, inclusive; about 60 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, or about 80 mg, inclusive; about 80 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, or about 100 mg, inclusive; about 100 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, or about 150 mg, inclusive; about 150 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, or about 200 mg, inclusive; about 200 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, or about 250 mg, inclusive; about 250 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, or about 300 mg, inclusive; about 300 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, or about 350 mg, inclusive; about 350 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, or about 400 mg, inclusive; about 400 mg to about 1,500 mg, about 1,400 mg, about 1,300 mg, about 1,200 mg, about 1,100 mg, about 1,000 mg, about 900 mg, about 800 mg, about 700 mg, about 600 mg, about 500 mg, or about 450 mg, inclusive; about 450 mg to about 1,500 mg, about 1,400 mg, about 1,300 mg, about 1,200 mg, about 1,100 mg, about 1,000 mg, about 900 mg, about 800 mg, about 700 mg, about 600 mg, or about 500 mg, inclusive; about 500 mg to about 1,500 mg, about 1,400 mg, about 1,300 mg, about 1,200 mg, about 1,100 mg, about 1,000 mg, about 900 mg, about 800 mg, about 700 mg, or about 600 mg, inclusive; about 600 mg to about 1,500 mg, about 1,400 mg, about 1,300 mg, about 1,200 mg, about 1,100 mg, about 1,000 mg, about 900 mg, about 800 mg, or about 700 mg, inclusive; about 700 mg to about 1,500 mg, about 1,400 mg, about 1,300 mg, about 1,200 mg, about 1,100 mg, about 1,000 mg, about 900 mg, or about 800 mg, inclusive; about 800 mg to about 1,500 mg, about 1,400 mg, about 1,300 mg, about 1,200 mg, about 1,100 mg, about 1,000 mg, or about 900 mg, inclusive; about 900 mg to about 1,500 mg, about 1,400 mg, about 1,300 mg, about 1,200 mg, about 1,100 mg, or about 1,000 mg, inclusive; about 1,000 mg to about 1,500 mg, about 1,400 mg, about 1,300 mg, about 1,200 mg, or about 1,100 mg, inclusive; about 1,100 mg to about 1,500 mg, about 1,400 mg, about 1,300 mg, or about 1,200 mg, inclusive; about 1,200 mg to about 1,500 mg, about 1,400 mg, or about 1,300 mg, inclusive; about 1,300 mg to about 1,500 mg or about 1,400 mg, inclusive; or about 1,400 mg to about 1,500 mg, inclusive, of a Lactobacillus species, a Bifidobacterium species, a Enterococcus species, or a Streptococcus thermophile, or the sum of two or more of a Lactobacillus species, a Bifidobacterium species, a Enterococcus species, and a Streptococcus thermophile, can be administered to a subject (e.g., any of the subjects described herein).

Any of the compositions provided herein can further include a carnosic acid (e.g., a therapeutically effective amount of a carnosic acid). Carnosic acid may be used as a preservative or antioxidant in any one of the compositions provided herein. In some examples, carnosic acid may provide an antimicrobial benefit to subjects ingesting the compositions provided herein. Carnosic acid may be obtained from Rosmarinus officinalis, Salvia officinalis, or a combination thereof. Carnosic acid may also be obtained as described elsewhere (e.g., U.S. Pat. No. 5,256,700). In some examples, carnosic acid can be purchased from a commercial vendor. For example, carnosic acid can be purchased from Cayman Chemical (Ann Arbor, Mich.).

A composition provided herein can contain any appropriate amount of carnosic acid. In some examples, about 0.1% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, about 1% w/w, or about 0.5% w/w, inclusive; about 0.2% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, about 1% w/w, or about 0.5% w/w, inclusive; about 0.5% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, or about 1% w/w, inclusive; about 1% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, or about 2% w/w, inclusive; about 2% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, or about 3% w/w, inclusive; about 3% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, or about 4% w/w, inclusive; about 4% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, or about 5% w/w inclusive; about 5% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, or about 6% w/w, inclusive; about 6% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, or about 8% w/w, inclusive; about 8% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, or about 10% w/w, inclusive; about 10% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, or about 12% w/w, inclusive; about 12% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, or about 14% w/w, inclusive; about 14% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, or about 16% w/w, inclusive; about 16% to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, or about 18% w/w, inclusive; about 18% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, or about 20% w/w, inclusive; about 20% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, or about 22% w/w, inclusive; about 22% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, or about 24% w/w, inclusive; about 24% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, or about 26% w/w, inclusive; about 26% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, or about 28% w/w, inclusive; about 28% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, or about 30% w/w, inclusive; about 30% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, or about 32% w/w, inclusive; about 32% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, inclusive; about 34% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, or about 36% w/w, inclusive; about 36% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, or about 38% w/w, inclusive; about 38% w/w to about 60% w/w, about 58% w/w, about 56% w/w, about 54% w/w, about 52% w/w, about 50% w/w, about 48% w/w, about 46% w/w, about 44% w/w, about 42% w/w, or about 40% w/w, inclusive; about 40% w/w to about 60% w/w, about 58% w/w, about 56% w/w, about 54% w/w, about 52% w/w, about 50% w/w, about 48% w/w, about 46% w/w, about 44% w/w, or about 42% w/w, inclusive; about 42% w/w to about 60% w/w, about 58% w/w, about 56% w/w, about 54% w/w, about 52% w/w, about 50% w/w, about 48% w/w, about 46% w/w, or about 44% w/w, inclusive; about 44% w/w to about 60% w/w, about 58% w/w, about 56% w/w, about 54% w/w, about 52% w/w, about 50% w/w, about 48% w/w, or about 46% w/w, inclusive; about 46% w/w to about 60% w/w, about 58% w/w, about 56% w/w, about 54% w/w, about 52% w/w, about 50% w/w, or about 48% w/w, inclusive; about 48% w/w to about 60% w/w, about 58% w/w, about 56% w/w, about 54% w/w, about 52% w/w, or about 50% w/w, inclusive; about 50% w/w to about 60% w/w, about 58% w/w, about 56% w/w, about 54% w/w, or about 52% w/w, inclusive; about 52% w/w to about 60% w/w, about 58% w/w, about 56% w/w, or about 54% w/w, inclusive; about 54% w/w to about 60% w/w, about 58% w/w, or about 56% w/w, inclusive; about 56% w/w to about 60% w/w, or about 58% w/w, inclusive; or about 58% w/w to about 60% w/w, inclusive, of the composition provided herein is a carnosic acid.

In some embodiments, no more than 0.5% w/w, no more than 1% w/w, no more than 2% w/w, no more than 4% w/w, no more than 5% w/w, no more than 6% w/w, no more than 8% w/w, no more than 10% w/w, no more than 12% w/w, no more than 14% w/w, no more than 15% w/w, no more than 16% w/w, no more than 18% w/w, no more than 20% w/w, no more than 22% w/w, no more than 24% w/w, no more than 25% w/w, no more than 26% w/w, no more than 28% w/w, no more than 30% w/w, no more than 32% w/w, no more than 34% w/w, no more than 35% w/w, no more than 36% w/w, no more than 38% w/w, no more than 40% w/w, no more than 42% w/w, no more than 44% w/w, no more than 45% w/w, no more than 46% w/w, no more than 48% w/w, no more than 50% w/w, no more than 52% w/w, no more than 54% w/w, no more than 55% w/w, no more than 56% w/w, no more than 58% w/w, or no more than 60% w/w of any of the compositions described herein is a carnosic acid.

In some examples, a single dose of any of the compositions provided herein can include from about 0.1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, about 1 mg, or about 0.5 mg, inclusive; about 0.5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg, inclusive; about 1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, or about 5 mg, inclusive; about 5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, or about 10 mg, inclusive; about 10 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, or about 20 mg, inclusive; about 20 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, or about 40 mg, inclusive; about 40 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, or about 60 mg, inclusive; about 60 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, or about 80 mg, inclusive; about 80 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, or about 100 mg, inclusive; about 100 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, or about 150 mg, inclusive; about 150 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, or about 200 mg, inclusive; about 200 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, or about 250 mg, inclusive; about 250 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, or about 300 mg, inclusive; about 300 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, or about 350 mg, inclusive; about 350 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, or about 400 mg, inclusive; about 400 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, or about 450 mg, inclusive; about 450 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, or about 500 mg, inclusive; about 500 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, or about 600 mg, inclusive; about 600 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, or about 700 mg, inclusive; about 700 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, or about 800 mg, inclusive; about 800 mg to about 1,800 mg, about 1,700 mg, about 1,600 mg, about 1,500 mg, about 1,400 mg, about 1,300 mg, about 1,200 mg, about 1,100 mg, about 1,000 mg, or about 900 mg, inclusive; about 900 mg to about 1,800 mg, about 1,700 mg, about 1,600 mg, about 1,500 mg, about 1,400 mg, about 1,300 mg, about 1,200 mg, about 1,100 mg, or about 1,000 mg, inclusive; about 1,000 mg to about 1,800 mg, about 1,700 mg, about 1,600 mg, about 1,500 mg, about 1,400 mg, about 1,300 mg, about 1,200 mg, or about 1,100 mg, inclusive; about 1,100 mg to about 1,800 mg, about 1,700 mg, about 1,600 mg, about 1,500 mg, about 1,400 mg, about 1,300 mg, about 1,200 mg, inclusive; about 1,200 mg to about 1,800 mg, about 1,700 mg, about 1,600 mg, about 1,500 mg, about 1,400 mg, or about 1,300 mg, inclusive; about 1,300 mg to about 1,800 mg, about 1,700 mg, about 1,600 mg, about 1,500 mg, or about 1,400 mg, inclusive; about 1,400 mg to about 1,800 mg, about 1,700 mg, about 1,600 mg, or about 1,500 mg, inclusive; about 1,500 mg to about 1,800 mg, about 1,700 mg, or about 1,600 mg, inclusive; about 1,600 mg to about 1,800 mg, or about 1,700 mg, inclusive; or about 1,700 mg to about 1,800 mg, inclusive, of a carnosic acid.

In some examples, a composition provided herein can be formulated to contain an amount of a carnosic acid such that a daily dose of about 0.1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, about 1 mg, or about 0.5 mg, inclusive; about 0.5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg, inclusive; about 1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, or about 5 mg, inclusive; about 5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, or about 10 mg, inclusive; about 10 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, or about 20 mg, inclusive; about 20 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, or about 40 mg, inclusive; about 40 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, or about 60 mg, inclusive; about 60 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, or about 80 mg, inclusive; about 80 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, or about 100 mg, inclusive; about 100 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, or about 150 mg, inclusive; about 150 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, or about 200 mg, inclusive; about 200 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, or about 250 mg, inclusive; about 250 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, or about 300 mg, inclusive; about 300 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, or about 350 mg, inclusive; about 350 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, or about 400 mg, inclusive; about 400 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, or about 450 mg, inclusive; about 450 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, or about 500 mg, inclusive; about 500 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, or about 600 mg, inclusive; about 600 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, or about 700 mg, inclusive; about 700 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, or about 800 mg, inclusive; about 800 mg to about 1,800 mg, about 1,700 mg, about 1,600 mg, about 1,500 mg, about 1,400 mg, about 1,300 mg, about 1,200 mg, about 1,100 mg, about 1,000 mg, or about 900 mg, inclusive; about 900 mg to about 1,800 mg, about 1,700 mg, about 1,600 mg, about 1,500 mg, about 1,400 mg, about 1,300 mg, about 1,200 mg, about 1,100 mg, or about 1,000 mg, inclusive; about 1,000 mg to about 1,800 mg, about 1,700 mg, about 1,600 mg, about 1,500 mg, about 1,400 mg, about 1,300 mg, about 1,200 mg, or about 1,100 mg, inclusive; about 1,100 mg to about 1,800 mg, about 1,700 mg, about 1,600 mg, about 1,500 mg, about 1,400 mg, about 1,300 mg, about 1,200 mg, inclusive; about 1,200 mg to about 1,800 mg, about 1,700 mg, about 1,600 mg, about 1,500 mg, about 1,400 mg, or about 1,300 mg, inclusive; about 1,300 mg to about 1,800 mg, about 1,700 mg, about 1,600 mg, about 1,500 mg, or about 1,400 mg, inclusive; about 1,400 mg to about 1,800 mg, about 1,700 mg, about 1,600 mg, or about 1,500 mg, inclusive; about 1,500 mg to about 1,800 mg, about 1,700 mg, or about 1,600 mg, inclusive; about 1,600 mg to about 1,800 mg, or about 1,700 mg, inclusive; or about 1,700 mg to about 1,800 mg, inclusive, of a carnosic acid is administered to a subject (e.g., any of the subjects described herein).

As described herein, a composition provided herein can include an effective amount of valerenic acid or a valerenic extract. Valerneic acid is a sesquiterpenoid constituent of the essential oil of the Valerian plant. Valerneic acid or a valerneic extract may also be obtained as described elsewhere (e.g., U.S. Pat. No. 6,383,526, filed Jul. 21, 2000). In some examples, valerneic acid or a valerneic extract can be obtained commercially. For example, valerneic acid or a valerneic extract can be obtained from Dow Chemical (Texas City, Tex.). Valerenic acid, or a valernic acid extract may provide an anti-inflammatory benefit to consumers ingesting the compositions provided herein.

In some examples, a composition can include valerneic acid or a valerneic extract. A composition provided herein can include any appropriate amount of valerneic acid or a valerneic extract. For example, about 0.1% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, about 1% w/w, or about 0.5% w/w, inclusive; about 0.2% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, about 1% w/w, or about 0.5% w/w, inclusive; about 0.5% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, about 2% w/w, or about 1% w/w, inclusive; about 1% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, about 3% w/w, or about 2% w/w, inclusive; about 2% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, about 4% w/w, or about 3% w/w, inclusive; about 3% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, about 5% w/w, or about 4% w/w, inclusive; about 4% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, about 6% w/w, or about 5% w/w inclusive; about 5% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, about 8% w/w, or about 6% w/w, inclusive; about 6% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, about 10% w/w, or about 8% w/w, inclusive; about 8% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, about 12% w/w, or about 10% w/w, inclusive; about 10% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, about 14% w/w, or about 12% w/w, inclusive; about 12% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, about 16% w/w, or about 14% w/w, inclusive; about 14% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, about 18% w/w, or about 16% w/w, inclusive; about 16% to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, about 20% w/w, or about 18% w/w, inclusive; about 18% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, about 22% w/w, or about 20% w/w, inclusive; about 20% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, about 24% w/w, or about 22% w/w, inclusive; about 22% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, about 26% w/w, or about 24% w/w, inclusive; about 24% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, about 28% w/w, or about 26% w/w, inclusive; about 26% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, about 30% w/w, or about 28% w/w, inclusive; about 28% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, about 32% w/w, or about 30% w/w, inclusive; about 30% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, or about 32% w/w, inclusive; about 32% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, about 36% w/w, about 34% w/w, inclusive; about 34% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, about 38% w/w, or about 36% w/w, inclusive; about 36% w/w to about 60% w/w, about 58% w/w, about 56% w/w, about 54% w/w, about 52% w/w, about 50% w/w, about 48% w/w, about 46% w/w, about 44% w/w, about 42% w/w, about 40% w/w, or about 38% w/w, inclusive; about 38% w/w to about 60% w/w, about 58% w/w, about 56% w/w, about 54% w/w, about 52% w/w, about 50% w/w, about 48% w/w, about 46% w/w, about 44% w/w, about 42% w/w, or about 40% w/w, inclusive; about 40% w/w to about 60% w/w, about 58% w/w, about 56% w/w, about 54% w/w, about 52% w/w, about 50% w/w, about 48% w/w, about 46% w/w, about 44% w/w, or about 42% w/w, inclusive; about 42% w/w to about 60% w/w, about 58% w/w, about 56% w/w, about 54% w/w, about 52% w/w, about 50% w/w, about 48% w/w, about 46% w/w, or about 44% w/w, inclusive; about 44% w/w to about 60% w/w, about 58% w/w, about 56% w/w, about 54% w/w, about 52% w/w, about 50% w/w, about 48% w/w, or about 46% w/w, inclusive; about 46% w/w to about 60% w/w, about 58% w/w, about 56% w/w, about 54% w/w, about 52% w/w, about 50% w/w, or about 48% w/w, inclusive; about 48% w/w to about 60% w/w, about 58% w/w, about 56% w/w, about 54% w/w, about 52% w/w, or about 50% w/w, inclusive; about 50% w/w to about 60% w/w, about 58% w/w, about 56% w/w, about 54% w/w, or about 52% w/w, inclusive; about 52% w/w to about 60% w/w, about 58% w/w, about 56% w/w, or about 54% w/w, inclusive; about 54% w/w to about 60% w/w, about 58% w/w, or about 56% w/w, inclusive; about 56% w/w to about 60% w/w, or about 58% w/w, inclusive; or about 58% w/w to about 60% w/w, inclusive, of any of the compositions provided herein is valerneic acid or a valerneic extract.

In some examples, no more than 1% w/w, no more than about 2% w/w, no more than 4% w/w, no more than 5% w/w, no more than 6% w/w, no more than 8% w/w, no more than 10% w/w, no more than 12% w/w, no more than 14% w/w, no more than 15% w/w, no more than 16% w/w, no more than 18% w/w, no more than 20% w/w, no more than about 22% w/w, no more than 24% w/w, no more than 25% w/w, no more than 26% w/w, no more than 28% w/w, no more than 30% w/w, no more than 32% w/w, no more than 34% w/w, no more than 35% w/w, no more than 36% w/w, no more than 38% w/w, no more than 40% w/w, no more than 42% w/w, no more than 44% w/w, no more than 45% w/w, no more than 46% w/w, no more than 48% w/w, no more than 50% w/w, no more than 52% w/w, no more than 54% w/w, no more than 56% w/w, no more than 58% w/w, or no more than 60% w/w, of any of the compositions provided herein is valerneic acid or a valerneic extract.

In some examples, a single dose of any of the compositions provided herein can include from about 0.1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, about 1 mg, or about 0.5 mg, inclusive; about 0.5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg, inclusive; about 1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, or about 5 mg, inclusive; about 5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, or about 10 mg, inclusive; about 10 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, or about 20 mg, inclusive; about 20 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, or about 40 mg, inclusive; about 40 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, or about 60 mg, inclusive; about 60 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, or about 80 mg, inclusive; about 80 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, or about 100 mg, inclusive; about 100 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, or about 150 mg, inclusive; about 150 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, or about 200 mg, inclusive; about 200 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, or about 250 mg, inclusive; about 250 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, or about 300 mg, inclusive; about 300 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, or about 350 mg, inclusive; about 350 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, or about 400 mg, inclusive; about 400 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, or about 450 mg, inclusive; about 450 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, or about 500 mg, inclusive; about 500 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, or about 600 mg, inclusive; about 600 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, or about 700 mg, inclusive; about 700 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, or about 800 mg, inclusive; about 800 mg to about 1,800 mg, about 1,700 mg, about 1,600 mg, about 1,500 mg, about 1,400 mg, about 1,300 mg, about 1,200 mg, about 1,100 mg, about 1,000 mg, or about 900 mg, inclusive; about 900 mg to about 1,800 mg, about 1,700 mg, about 1,600 mg, about 1,500 mg, about 1,400 mg, about 1,300 mg, about 1,200 mg, about 1,100 mg, or about 1,000 mg, inclusive; about 1,000 mg to about 1,800 mg, about 1,700 mg, about 1,600 mg, about 1,500 mg, about 1,400 mg, about 1,300 mg, about 1,200 mg, or about 1,100 mg, inclusive; about 1,100 mg to about 1,800 mg, about 1,700 mg, about 1,600 mg, about 1,500 mg, about 1,400 mg, about 1,300 mg, about 1,200 mg, inclusive; about 1,200 mg to about 1,800 mg, about 1,700 mg, about 1,600 mg, about 1,500 mg, about 1,400 mg, or about 1,300 mg, inclusive; about 1,300 mg to about 1,800 mg, about 1,700 mg, about 1,600 mg, about 1,500 mg, or about 1,400 mg, inclusive; about 1,400 mg to about 1,800 mg, about 1,700 mg, about 1,600 mg, or about 1,500 mg, inclusive; about 1,500 mg to about 1,800 mg, about 1,700 mg, or about 1,600 mg, inclusive; about 1,600 mg to about 1,800 mg, or about 1,700 mg, inclusive; or about 1,700 mg to about 1,800 mg, inclusive, of valerneic acid or a valerneic extract.

In some examples, a composition provided herein can be formulated to contain an amount of valerneic acid or a valerneic extract such that a daily dose of about 0.1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, about 1 mg, or about 0.5 mg, inclusive; about 0.5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg, inclusive; about 1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, or about 5 mg, inclusive; about 5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, or about 10 mg, inclusive; about 10 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, or about 20 mg, inclusive; about 20 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, or about 40 mg, inclusive; about 40 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, or about 60 mg, inclusive; about 60 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, or about 80 mg, inclusive; about 80 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, or about 100 mg, inclusive; about 100 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, or about 150 mg, inclusive; about 150 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, or about 200 mg, inclusive; about 200 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, or about 250 mg, inclusive; about 250 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, or about 300 mg, inclusive; about 300 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, or about 350 mg, inclusive; about 350 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, or about 400 mg, inclusive; about 400 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, or about 450 mg, inclusive; about 450 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, or about 500 mg, inclusive; about 500 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, or about 600 mg, inclusive; about 600 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, or about 700 mg, inclusive; about 700 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, or about 800 mg, inclusive; about 800 mg to about 1,800 mg, about 1,700 mg, about 1,600 mg, about 1,500 mg, about 1,400 mg, about 1,300 mg, about 1,200 mg, about 1,100 mg, about 1,000 mg, or about 900 mg, inclusive; about 900 mg to about 1,800 mg, about 1,700 mg, about 1,600 mg, about 1,500 mg, about 1,400 mg, about 1,300 mg, about 1,200 mg, about 1,100 mg, or about 1,000 mg, inclusive; about 1,000 mg to about 1,800 mg, about 1,700 mg, about 1,600 mg, about 1,500 mg, about 1,400 mg, about 1,300 mg, about 1,200 mg, or about 1,100 mg, inclusive; about 1,100 mg to about 1,800 mg, about 1,700 mg, about 1,600 mg, about 1,500 mg, about 1,400 mg, about 1,300 mg, about 1,200 mg, inclusive; about 1,200 mg to about 1,800 mg, about 1,700 mg, about 1,600 mg, about 1,500 mg, about 1,400 mg, or about 1,300 mg, inclusive; about 1,300 mg to about 1,800 mg, about 1,700 mg, about 1,600 mg, about 1,500 mg, or about 1,400 mg, inclusive; about 1,400 mg to about 1,800 mg, about 1,700 mg, about 1,600 mg, or about 1,500 mg, inclusive; about 1,500 mg to about 1,800 mg, about 1,700 mg, or about 1,600 mg, inclusive; about 1,600 mg to about 1,800 mg, or about 1,700 mg, inclusive; or about 1,700 mg to about 1,800 mg, inclusive, of the valerneic acid or a valerneic extract can be administered to a subject (e.g., any of the subjects described herein).

In some examples, a composition provided herein can further include one or more (e.g., two, three, four, five, six, seven, eight, nine, ten, eleven, twelve, or thirteen) of Vitamin E, tocopherol, tocotrienol, Vitamin A, carotene, lutein, astaxanthin, CoQ10, Vitamin C, folate, uric acid, Vitamin B12, and folic acid. In some examples, a single dose of any of the compositions provided herein can include about 0.1 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 IU, about 900 IU, about 850 IU, about 800 IU, about 750 IU, about 700 IU, about 650 IU, about 600 IU, about 550 IU, about 500 IU, about 450 IU, about 400 IU, about 350 IU, about 300 IU, about 250 IU, about 200 IU, about 150 IU, about 100 IU, about 80 IU, about 60 IU, about 40 IU, about 20 IU, about 10 IU, about 5 IU, or about 1 IU, inclusive; about 1 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 IU, about 900 IU, about 850 IU, about 800 IU, about 750 IU, about 700 IU, about 650 IU, about 600 IU, about 550 IU, about 500 IU, about 450 IU, about 400 IU, about 350 IU, about 300 IU, about 250 IU, about 200 IU, about 150 IU, about 100 IU, about 80 IU, about 60 IU, about 40 IU, about 20 IU, about 10 IU, or about 5 IU, inclusive; about 5 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 IU, about 900 IU, about 850 IU, about 800 IU, about 750 IU, about 700 IU, about 650 IU, about 600 IU, about 550 IU, about 500 IU, about 450 IU, about 400 IU, about 350 IU, about 300 IU, about 250 IU, about 200 IU, about 150 IU, about 100 IU, about 80 IU, about 60 IU, about 40 IU, about 20 IU, or about 10 IU, inclusive; about 10 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 IU, about 900 IU, about 850 IU, about 800 IU, about 750 IU, about 700 IU, about 650 IU, about 600 IU, about 550 IU, about 500 IU, about 450 IU, about 400 IU, about 350 IU, about 300 IU, about 250 IU, about 200 IU, about 150 IU, about 100 IU, about 80 IU, about 60 IU, about 40 IU, or about 20 IU, inclusive; about 20 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 IU, about 900 IU, about 850 IU, about 800 IU, about 750 IU, about 700 IU, about 650 IU, about 600 IU, about 550 IU, about 500 IU, about 450 IU, about 400 IU, about 350 IU, about 300 IU, about 250 about 200 IU, about 150 IU, about 100 IU, about 80 IU, about 60 IU, or about 40 IU, inclusive; about 25 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 IU, about 900 IU, about 850 IU, about 800 IU, about 750 IU, about 700 IU, about 650 IU, about 600 IU, about 550 IU, about 500 IU, about 450 IU, about 400 IU, about 350 IU, about 300 IU, about 250 IU, about 200 IU, about 150 IU, about 100 IU, about 80 IU, about 60 IU, or about 40 IU, inclusive; about 40 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 IU, about 900 IU, about 850 IU, about 800 IU, about 750 IU, about 700 IU, about 650 IU, about 600 IU, about 550 IU, about 500 IU, about 450 IU, about 400 IU, about 350 IU, about 300 IU, about 250 IU, about 200 IU, about 150 IU, about 100 IU, about 80 IU, or about 60 IU, inclusive; about 60 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 IU, about 900 IU, about 850 IU, about 800 IU, about 750 IU, about 700 IU, about 650 IU, about 600 IU, about 550 IU, about 500 IU, about 450 IU, about 400 IU, about 350 IU, about 300 IU, about 250 IU, about 200 IU, about 150 IU, about 100 IU, or about 80 IU, inclusive; about 80 IU to about 2,000 IU, about 1,900 IU, about 1,800 IU, about 1,700 IU, about 1,600 IU, about 1,500 IU, about 1,400 IU, about 1,300 IU, about 1,200 about 1,100 IU, about 1,000 IU, about 950 IU, about 900 IU, about 850 IU, about 800 IU, about 750 IU, about 700 IU, about 650 IU, about 600 IU, about 550 IU, about 500 IU, about 450 IU, about 400 IU, about 350 IU, about 300 IU, about 250 IU, about 200 IU, about 150 IU, or about 100 IU, inclusive; about 100 IU to about 2,000 IU, about 1,900 IU, about 1,800 IU, about 1,700 IU, about 1,600 IU, about 1,500 IU, about 1,400 about 1,300 IU, about 1,200 IU, about 1,100 IU, about 1,000 IU, about 950 IU, about 900 IU, about 850 IU, about 800 IU, about 750 IU, about 700 IU, about 650 IU, about 600 IU, about 550 IU, about 500 IU, about 450 IU, about 400 IU, about 350 IU, about 300 IU, about 250 IU, about 200 IU, or about 150 IU, inclusive; about 150 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 IU, about 900 IU, about 850 IU, about 800 IU, about 750 IU, about 700 IU, about 650 IU, about 600 IU, about 550 IU, about 500 IU, about 450 IU, about 400 IU, about 350 IU, about 300 IU, about 250 IU, or about 200 IU, inclusive; about 200 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 IU, about 900 IU, about 850 IU, about 800 IU, about 750 IU, about 700 IU, about 650 IU, about 600 IU, about 550 IU, about 500 IU, about 450 IU, about 400 IU, about 350 IU, about 300 IU, or about 250 IU, inclusive; about 250 IU to about 2,000 IU, about 1,900 IU, about 1,800 IU, about 1,700 IU, about 1,600 IU, about 1,500 IU, about 1,400 about 1,300 IU, about 1,200 IU, about 1,100 IU, about 1,000 IU, about 950 IU, about 900 IU, about 850 IU, about 800 IU, about 750 IU, about 700 IU, about 650 IU, about 600 IU, about 550 IU, about 500 IU, about 450 IU, about 400 IU, about 350 IU, or about 300 IU, inclusive; about 300 IU to about 2,000 IU, about 1,900 IU, about 1,800 IU, about 1,700 IU, about 1,600 IU, about 1,500 IU, about 1,400 IU, about 1,300 IU, about 1,200 about 1,100 IU, about 1,000 IU, about 950 IU, about 900 IU, about 850 IU, about 800 IU, about 750 IU, about 700 IU, about 650 IU, about 600 IU, about 550 IU, about 500 IU, about 450 IU, about 400 IU, or about 350 IU, inclusive; about 350 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 about 900 IU, about 850 IU, about 800 IU, about 750 IU, about 700 IU, about 650 IU, about 600 IU, about 550 IU, about 500 IU, about 450 IU, about 400 IU, inclusive; about 400 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 IU, about 900 IU, about 850 IU, about 800 IU, about 750 about 700 IU, about 650 IU, about 600 IU, about 550 IU, about 500 IU, or about 450 IU, inclusive; about 450 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 IU, about 900 IU, about 850 IU, about 800 IU, about 750 IU, about 700 IU, about 650 IU, about 600 IU, about 550 IU, or about 500 inclusive; about 500 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 IU, about 900 IU, about 850 IU, about 800 IU, about 750 IU, about 700 IU, about 650 IU, about 600 IU, or about 550 IU, inclusive; about 550 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 IU, about 900 IU, about 850 IU, about 800 IU, about 750 IU, about 700 IU, about 650 IU, or about 600 IU, inclusive; about 600 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 IU, about 900 IU, about 850 IU, about 800 IU, about 750 IU, about 700 IU, or about 650 IU, inclusive; about 650 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 IU, about 900 IU, about 850 IU, about 800 IU, about 750 IU, or about 700 IU, inclusive; about 700 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 IU, about 900 IU, about 850 IU, about 800 IU, or about 750 IU, inclusive; about 750 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 about 950 IU, about 900 IU, about 850 IU, or about 800 IU, inclusive; about 800 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, about 950 IU, about 900 IU, or about 850 IU, inclusive; about 850 IU to about 2,000 IU, about 1,900 IU, about 1,800 IU, about 1,700 IU, about 1,600 IU, about 1,500 about 1,400 IU, about 1,300 IU, about 1,200 IU, about 1,100 IU, about 1,000 IU, about 950 IU, or about 900 IU, inclusive; about 900 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, about 1,000 IU, or about 950 IU, inclusive; about 950 IU to about 2,000 IU, about 1,900 IU, about 1,800 IU, about 1,700 IU, about 1,600 IU, about 1,500 IU, about 1,400 IU, about 1,300 IU, about 1,200 IU, about 1,100 IU, or about 1,000 IU, inclusive; about 1,000 IU to about 2,000 IU, about 1,900 IU, about 1,800 IU, about 1,700 IU, about 1,600 IU, about 1,500 IU, about 1,400 IU, about 1,300 IU, about 1,200 IU, or about 1,100 IU, inclusive; about 1,100 IU to about 2,000 IU, about 1,900 IU, about 1,800 IU, about 1,700 IU, about 1,600 IU, about 1,500 IU, about 1,400 IU, about 1,300 IU, or about 1,200 IU, inclusive; about 1,200 IU to about 2,000 IU, about 1,900 IU, about 1,800 IU, about 1,700 IU, about 1,600 IU, about 1,500 IU, about 1,400 IU, or about 1,300 IU, inclusive; about 1,300 IU to about 2,000 IU, about 1,900 IU, about 1,800 IU, about 1,700 IU, about 1,600 IU, about 1,500 IU, or about 1,400 IU, inclusive; about 1,400 IU to about 2,000 IU, about 1,900 IU, about 1,800 IU, about 1,700 IU, about 1,600 IU, or about 1,500 IU, inclusive; about 1,500 IU to about 2,000 IU, about 1,900 IU, about 1,800 IU, about 1,700 IU, or about 1,600 IU, inclusive; about 1,600 IU to about 2,000 IU, about 1,900 IU, about 1,800 IU, or about 1,700 IU, inclusive; about 1,700 IU to about 2,000 IU, about 1,900 IU, or about 1,800 IU, inclusive; about 1,800 IU to about 2,000 IU or about 1,900 IU, inclusive; or about 1,900 IU to about 2,000 IU, inclusive, of any of one or more of Vitamin E, tocopherol, tocotrienol, Vitamin A, carotene, lutein, astaxanthin, CoQ10, Vitamin C, folate, and uric acid.

In some examples, a single dose of any of the compositions provided herein can include about 0.1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, about 1 mg, or about 0.5 mg, inclusive; about 0.5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg, inclusive; about 1 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, or about 5 mg, inclusive; about 5 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, or about 10 mg, inclusive; about 10 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, about 40 mg, or about 20 mg, inclusive; about 20 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, about 60 mg, or about 40 mg, inclusive; about 40 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, about 80 mg, or about 60 mg, inclusive; about 60 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, about 100 mg, or about 80 mg, inclusive; about 80 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, about 150 mg, or about 100 mg, inclusive; about 100 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, about 200 mg, or about 150 mg, inclusive; about 150 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, about 250 mg, or about 200 mg, inclusive; about 200 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, about 300 mg, or about 250 mg, inclusive; about 250 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, about 350 mg, or about 300 mg, inclusive; about 300 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, about 400 mg, or about 350 mg, inclusive; about 350 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, about 450 mg, or about 400 mg, inclusive; about 400 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, about 500 mg, or about 450 mg, inclusive; about 450 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, about 600 mg, or about 500 mg, inclusive; about 500 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, about 700 mg, or about 600 mg, inclusive; about 600 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, about 800 mg, or about 700 mg, inclusive; about 700 mg to about 1,800 mg, about 1,700 mg, about 1,600 mg, about 1,500 mg, about 1,400 mg, about 1,300 mg, about 1,200 mg, about 1,100 mg, about 1,000 mg, about 900 mg, or about 800 mg, inclusive; about 800 mg to about 1,800 mg, about 1,700 mg, about 1,600 mg, about 1,500 mg, about 1,400 mg, about 1,300 mg, about 1,200 mg, about 1,100 mg, about 1,000 mg, or about 900 mg, inclusive; about 900 mg to about 1,800 mg, about 1,700 mg, about 1,600 mg, about 1,500 mg, about 1,400 mg, about 1,300 mg, about 1,200 mg, about 1,100 mg, or about 1,000 mg, inclusive; about 1,000 mg to about 1,800 mg, about 1,700 mg, about 1,600 mg, about 1,500 mg, about 1,400 mg, about 1,300 mg, about 1,200 mg, or about 1,100 mg, inclusive; about 1,100 mg to about 1,800 mg, about 1,700 mg, about 1,600 mg, about 1,500 mg, about 1,400 mg, about 1,300 mg, about 1,200 mg, inclusive; about 1,200 mg to about 1,800 mg, about 1,700 mg, about 1,600 mg, about 1,500 mg, about 1,400 mg, or about 1,300 mg, inclusive; about 1,300 mg to about 1,800 mg, about 1,700 mg, about 1,600 mg, about 1,500 mg, or about 1,400 mg, inclusive; about 1,400 mg to about 1,800 mg, about 1,700 mg, about 1,600 mg, or about 1,500 mg, inclusive; about 1,500 mg to about 1,800 mg, about 1,700 mg, or about 1,600 mg, inclusive; about 1,600 mg to about 1,800 mg, or about 1,700 mg, inclusive; or about 1,700 mg to about 1,800 mg, inclusive, of any of one or more of Vitamin E, tocopherol, tocotrienol, Vitamin A, carotene, lutein, astaxanthin, CoQ10, Vitamin C, folate, and uric acid.

Any of the compositions provided herein can be formulated in an oral dosage form. In some examples, any of the compositions provided herein can be formulated in the form of a liquid, semi-liquid, semi-solid, syrup, slurry, chewable form, solution, dispersion, suspension, tablet, pellet, pill, granule, emulsion, gel, a plurality of beads encapsulated in a capsule, powder, suspension, cream, mist, atomized vapor, aerosol, soft gelatin capsule, hard gelatin capsule, a patch, a gel, a confectionary, a shake, a bar, or a supplemented food. In some examples, any of the compositions provided herein can be formulated for intravenous delivery (e.g., by injection) or inhalation (e.g., by nasal spray).

In some examples, any of the compositions provided herein can be an oral dosage form that is a tablet, a capsule, or a pellet. Some embodiments of any of the compositions provided herein may further include suitable excipients, flavorings, colorants, and/or other ingredients. In some examples, tablets or capsules can be prepared with pharmaceutically acceptable excipients such as binding agents, fillers, lubricants, disintegrants, and/or wetting agents.

In some examples, solid forms of any of the compositions provided herein can be obtained by adding a solid excipient to compositions provided herein, and optionally grinding a resulting mixture and processing the mixture of granules, after adding suitable auxiliaries. Suitable excipients include, e.g., fillers such as sugars (e.g., lactose, sucrose, mannitol, and/or sorbitol) or cellulose preparations (e.g., maize starch, wheat starch, rice starch, potato starch, gelatin, gum tragacanth, methyl cellulose, hydroxypropylmethyl-cellulose, sodium carboxymethylcellulose, polyvinylpyrrolidone (PVP)), or combinations thereof. Any of the compositions described herein can be prepared by uniformly and intimately admixing the active ingredient with liquid carriers or finely divided solid carriers or both, and optionally shaping the product into the desired presentation. For example, a tablet can be prepared by compression or molding, optionally with one or more accessory ingredients. Compressed tablets can be prepared by compressing the active agents disclosed herein in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

In some examples, the compositions provided herein can be in liquid form such as an aqueous solution, suitably flavored syrup, aqueous or oil suspension, and/or flavored emulsion with edible oils (e.g., cottonseed oil, sesame oil, coconut oil, and/or peanut oil). Suitable dispersing or suspending agents for aqueous suspensions can include synthetic natural gums, such as tragacanth, acacia, alginate, dextran, sodium carboxymethyl cellulose, methylcellulose, polyvinylpyrrolidone, and/or gelatin.

Liquid preparations for oral administration can take the form of, for example, solutions, syrups, or suspensions, or as a dry product for reconstitution with water or other suitable vehicles before use. Such liquid preparations can be prepared by using suitable additives such as suspending agents (e.g., sorbitol syrup, methyl cellulose, and/or hydrogenated edible fats); emulsifying agents (e.g., lecithin or acacia); non-aqueous vehicles (e.g., almond oil, oily esters, and/or ethyl alcohol); preservatives (e.g., methyl or propyl p-hydroxybenzoates, and/or sorbic acid); artificial or natural colors; and/or sweeteners.

Compositions provided herein can include anhydrous compositions and dosage forms including an active ingredient, since water may degrade some compounds in the composition. Anhydrous compositions are defined as compositions including less than 5% w/w (e.g., less than 3% w/w, less than 1% w/w, less than 0.1% w/w, or less than 0.01% w/w) of water. Anhydrous compositions and dosage forms of the compositions provided herein can be prepared using anhydrous or low-moisture-containing ingredients and under low moisture or low humidity conditions. An anhydrous composition can be prepared and stored such that its anhydrous nature is maintained. Accordingly, anhydrous compositions can be packaged using materials known to prevent exposure to water, such that they can be included in suitable formulary kits. Examples of suitable packaging include, but are not limited to, hermetically sealed foils, plastic, or the like, unit dose containers, blister packs, and strip packs.

In some examples, compositions provided herein can contain a pharmaceutically acceptable carrier for administration to a mammal, including, without limitation, sterile aqueous, or non-aqueous solutions, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Pharmaceutically acceptable carriers also can include physiologically acceptable aqueous vehicles (e.g., physiological saline) or other carriers appropriate for oral administration.

Any of the active agents disclosed herein can be combined with a suitable carrier. Suitable carriers can include, but are not limited to, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, disintegrating agents, and combinations thereof. More specifically, suitable carriers can include: (1) sugars, e.g., lactose, glucose, and/or sucrose; (2) starches, e.g., corn starch and/or potato starch; (3) cellulose, and its derivatives, e.g., sodium carboxymethyl cellulose, ethyl cellulose, and/or cellulose acetate; (4) powdered tragacanth; (5) malt; (6) gelatin; (7) talc; (8) excipients, e.g., cocoa butter and/or suppository waxes; (9) oils, e.g., peanut oil, cottonseed oil, safflower oil, sesame oil, olive oil, corn oil, and/or soybean oil; (10) glycols, e.g., a propylene glycol; (11) polyols, e.g., glycerin, sorbitol, mannitol, and/or polyethylene glycol; (12) esters, e.g., ethyl oleate and/or ethyl laurate; (13) agar; (14) buffering agents, e.g., magnesium hydroxide and/or aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Binders can be suitable for use in dosage forms in some examples. Suitable binders can include, but are not limited to, corn starch, potato starch, and other starches, gelatin, natural and synthetic gums, e.g., acacia, sodium alginate, alginic acid, other alginates, powdered tragacanth, guar gum, cellulose and its derivatives (e.g., ethyl cellulose, cellulose acetate, carboxymethyl cellulose calcium, and/or sodium carboxymethyl cellulose), polyvinyl pyrrolidone, methyl cellulose, pre-gelatinized starch, hydroxypropyl methyl cellulose, microcrystalline cellulose, and mixtures thereof.

Lubricants which can be used to form compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and/or soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant can optionally be added, in an amount of less than about 1% w/w of the composition. Lubricants can be also be used in conjunction with tissue barriers which may include, but are not limited to, polysaccharides, polyglycans, seprafilm, interceed, and/or hyaluronic acid.

Disintegrants can be included to allow the oral dosage form to disintegrate when exposed to an aqueous environment. Too much of a disintegrant can produce tablets which can disintegrate in the bottle. Too little can be insufficient for disintegration to occur and can thus alter the rate and extent of release of the active ingredient(s) from the dosage form. Thus, a sufficient amount of disintegrant that is neither too little nor too much to detrimentally alter the release of the active agent(s) can be used to form the dosage forms of the compounds disclosed herein. The amount of disintegrant used can vary based upon the type of formulation and mode of administration. In some examples, the amount of disintegrant can range from about 0.5% w/w to about 15% w/w of the composition, or about 1% w/w to about 5% w/w in any of the compositions provided herein. Disintegrants that can be used to form compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, or gums, or any mixture thereof.

Examples of suitable fillers for use in the compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

For aqueous suspensions suitable for oral administration, the active agents provided herein can be combined with various sweetening or flavoring agents, colorants, emulsifying agents, suspending agents, diluents (e.g., water, ethanol, propylene glycol, and glycerin), and any combination thereof.

In some examples, the compositions provided herein can be provided in a soft gelatin capsule or hard gelatin capsule form (which also referred to as a two-piece capsule). Soft and hard capsules may be made with a hydrocolloid that forms a colloidal solution in water, e.g., gelatin, and an optional plasticizer, e.g., glycerol or sorbitol. Gelatin exhibits a unique combination of useful properties, such as water solubility, solution viscosity, thermally-reversible gelation properties and an ability to form strong, clear, and flexible, high-gloss films. Gelatin can be used in the production of hard and soft capsules because gelatin can form clear, flexible, glossy capsule walls. The gelatin capsules can provide a material that will dissolve in the stomach. In some examples, hard gelatin capsules can be made by mixing the active agent with an inert solid diluent, e.g., calcium carbonate, calcium phosphate, and/or kaolin. In some examples, soft gelatin capsules can be made by mixing the active agent with water or an oil medium, e.g., peanut oil, liquid paraffin, and/or olive oil. Capsules can be made using a dip molding process. A dip molding process can comprise the steps of dipping molds pins into a hot solution of gelatin, removing the pins from the gelatin solution, allowing the gelatin solution attached on pins to set by cooling, drying, and stripping formed shells from the pins.

A preferred edible dosage form for the compositions provided herein is a hard capsule. Hard capsules are easy to swallow and may provide a tasteless shell. Some hard capsules may be opened up and sprinkled onto food or into water/juice if a subject has difficulty swallowing.

Preparations for oral administration can be suitably formulated to give controlled-release of one or more active ingredients. In some examples, the compositions provided herein can be in the form of tablets, capsules, or pellets that include a coating (e.g., a polymer or polysaccharide-based coating with or without plasticizers and/or pigments). For example, in some examples, a composition provided herein can include an enteric coating for inhibiting release of one or more active agents from a capsule, tablet, or pellet. For example, in some examples, a composition provided herein can be in the form of a capsule having a coating that dissolves at a suitable pH (e.g., a pH of about 1 to about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, or about 1.2, inclusive; about 1.1 to about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, or about 1.3, inclusive; about 1.2 to about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, or about 1.4, inclusive; about 1.3 to about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, or about 1.5, inclusive; about 1.4 to about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, or about 1.6, inclusive; about 1.5 to about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, or about 1.7, inclusive; about 1.6 to about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, or about 1.8, inclusive; about 1.7 to about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, or about 1.9, inclusive; about 1.8 to about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, or about 2.0, inclusive; about 1.9 to about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, inclusive; about 2.0 to about 3.5, about 3.4, about 3.3, about 3.2, about 3.1, about 3.0, about 2.9, about 2.8, about 2.7, about 2.6, about 2.5, about 2.4, about 2.3, or about 2.2, inclusive; about 2.1 to about 3.5, about 3.4, about 3.3, about 3.2, about 3.1, about 3.0, about 2.9, about 2.8, about 2.7, about 2.6, about 2.5, about 2.4, or about 2.3, inclusive; about 2.2 to about 3.5, about 3.4, about 3.3, about 3.2, about 3.1, about 3.0, about 2.9, about 2.8, about 2.7, about 2.6, about 2.5, or about 2.4, inclusive; about 2.3 to about 3.5, about 3.4, about 3.3, about 3.2, about 3.1, about 3.0, about 2.9, about 2.8, about 2.7, about 2.6, or about 2.5, inclusive; about 2.4 to about 3.5, about 3.4, about 3.3, about 3.2, about 3.1, about 3.0, about 2.9, about 2.8, about 2.7, or about 2.6, inclusive; about 2.5 to about 3.5, about 3.4, about 3.3, about 3.2, about 3.1, about 3.0, about 2.9, about 2.8, or about 2.7, inclusive; about 2.6 to about 3.5, about 3.4, about 3.3, about 3.2, about 3.1, about 3.0, about 2.9, or about 2.8, inclusive; about 2.7 to about 3.5, about 3.4, about 3.3, about 3.2, about 3.1, about 3.0, about 2.9, inclusive; about 2.8 to about 3.5, about 3.4, about 3.3, about 3.2, about 3.1, or about 3.0, inclusive; about 2.9 to about 3.5, about 3.4, about 3.3, about 3.2, or about 3.1, inclusive; about 3.0 to about 3.5, about 3.4, about 3.3, or about 3.2, inclusive; about 3.1 to about 3.5, about 3.4, or about 3.3, inclusive; about 3.2 to about 3.5 or about 3.4, inclusive; or about 3.3 to about 3.5, inclusive). Suitable enteric coatings can include one or more cellulosic materials, including but not limited to, ethylcellulose, hydroxypropyl methylcellulose, and hydroxypropylmethyl cellulose phthalate. In some examples, tablets or capsules can be coated with a methacrylic acid copolymer (e.g., Eudragit L100-55 or Eudragit S100) for release beyond the stomach (e.g., in the intestine, colon, or both). In some examples, the coating includes a time delay material such as glyceryl monostearate or glyceryl distearate. Tablets or capsules may be, e.g., coated by standard aqueous or nonaqueous techniques. In some embodiments, the enteric coating can include one or more of the active ingredients provided herein. In some examples, the enteric coating does not contain any of the active ingredients provided herein (e.g., less than 0.001% w/w of the composition).

Liquid preparations for oral administration can take the form of, for example, solutions, syrups, or suspension, or they can be presented as a dry product for constitution with saline or any other suitable liquid vehicle before use. In some examples, liquid preparations can contain pharmaceutically acceptable additives such as suspending agents, emulsifying agents, non-aqueous vehicles, preservatives, buffer salts, flavoring agents, coloring agents, and sweetening agents, as appropriate.

The composition provided herein can optionally include one or more additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof. A non-exhaustive list of examples of excipients includes monoglycerides, magnesium stearate, modified food starch, gelatin, microcrystalline cellulose, glycerin, stearic acid, silica, yellow beeswax, lecithin, hydroxypropylcellulose, croscarmellose sodium, crospovidone, and combinations thereof.

In some embodiments, the compositions can be formulated in a food composition. For example, the compositions can be a beverage or other liquids, solid food, semi-solid food, with or without a food carrier. For example, the compositions can include a black tea supplemented with any of the compositions described herein. The composition can be a dairy product supplemented with any of the compositions described herein. In some embodiments, the compositions can be formulated in a food composition. For example, the compositions can include a beverage, solid food, semi-solid food, or a food carrier.

In some embodiments, liquid food carriers, such as in the form of beverages, such as supplemented juices, coffees, teas, sodas, flavored waters, shakes, and the like can be used. For example, the beverage can comprise the formulation as well as a liquid component, such as various deodorant or natural carbohydrates present in conventional beverages. Examples of natural carbohydrates include, but are not limited to, monosaccharides, e.g., glucose and/or fructose; disaccharides, e.g., maltose and/or sucrose; conventional sugars, e.g., dextrin and/or cyclodextrin; and sugar alcohols, e.g., xylitol and/or erythritol. Natural deodorant, e.g., taumatin, stevia extract, levaudioside A, glycyrrhizin, and/or synthetic deodorant, e.g., saccharin and/or aspartame can also be included in the compositions described herein. Agents such as flavoring agents, coloring agents, and others can also be included in any of the compositions described herein. For example, pectic acid and the salt thereof, alginic acid and the salt thereof, organic acid, protective colloidal adhesive, pH controlling agent, stabilizer, a preservative, glycerin, alcohol, or carbonizing agents can also be included in any of the compositions described herein. Fruit and vegetables can also be used in preparing any of the compositions described herein.

In some examples, the compositions provided herein can be a snack bar. An exemplary snack bar can be a chocolate bar, a granola bar, a protein bar, or a trail mix bar. In yet another embodiment, the compositions provided herein are formulated to have suitable and desirable taste, texture, and viscosity for consumption. Any suitable food carrier can be used in the present food compositions. Suitable food carriers can include, but are not limited, to food bars (e.g., granola bars, protein bars, candy bars, etc.), cereal products (e.g., oatmeal, breakfast cereals, granola, etc.), bakery products (e.g., bread, donuts, crackers, bagels, pastries, cakes, etc.), beverages (e.g., milk-based beverage, sports drinks, fruit juices, alcoholic beverages, bottled waters, and shakes), pastas, grains (e.g., rice, corn, oats, rye, wheat, flour, etc.), egg products, snacks (e.g., candy, chips, gum, chocolate, etc.), meats, fruits, and/or vegetables. In some examples, the food carriers can reduce or mask an undesirable taste (e.g., bitterness). In some examples, the compositions provided herein can be a beverage, such as a supplemented juice, coffee, tea, and the like. In some examples, solid food carriers can include food compositions that provide a form of meal replacement, such as a supplemented snack bar, pasta, bread, and the like. In some examples, semi-solid food carriers can be used in compositions provided herein in the form of gums, chewy candies, or snacks, and the like.

In some examples, the compositions provided herein can be in the form of a capsule or tablet containing a therapeutically effective amount of zeolite as the only active ingredient. In some examples, compositions provided herein can be in the form of a capsule or tablet containing a therapeutically effective amount of zeolite and one or more additional active ingredients, such as, but not limited to, valerenic acid, carnosic acid, vitamin E, and combinations thereof.

The dosing of any of the compositions provided herein can be administered about, less than about, or more than about 1, 2, 3, 4, 5, 6, 7, 8, 9, 10 or more times a day. A subject can receive dosing for a period of about, less than about, or greater than about 1, 2, 3, 4, 5, 6, 7, 8, 9, 10, 11, 12, 13, 14 or more days, weeks or months. A dose or a unit dose can be a fraction of the daily dose, such as the daily dose divided by the number of doses or unit doses to be administered per day. A dose or unit dose can be a fraction of the daily dose that is the daily dose divided by the number of doses or unit doses to be administered per day and further divided by the number of unit doses or doses (e.g. tablets) per administration. The number of doses or unit doses per administration can be about, less than about, or more than about 1, 2, 3, 4, 5, 6, 7, 8, 9, 10, or more. The number of doses or unit doses per day can be about, less than about, or more than about 1, 2, 3, 4, 5, 6, 7, 8, 9, 10, or more. The number of doses or unit doses per day can be determined by dividing the daily dose by the dose or unit dose, and can be about, less than about, or more than about 1, 2, 3, 4, 5, 6, 7, 8, 9, 10, 11, 12, 13, 14, 15, 6, 17, 18, 19, 20, or more doses or unit doses per day. For example, a dose or unit dose can be about ½, ⅓, ¼, ⅕, ⅙, 1/7, ⅛, 1/9, 1/10 of the daily dose. A dose or unit dose can be about one-third of the daily dose and administered to the subject three times daily. A dose or unit dose can be about one-half of the daily dose and administered to the subject twice daily. A dose or unit dose can be about one-fourth of the daily dose with two unit doses administered to the subject twice daily.

In some embodiments, a dose or unit dose is administered as one unit dose two times per day. A dose or unit dose can include more than one capsule, tablet, vial, or entity. Compositions disclosed herein can further include a flavorant and can be a solid, liquid, gel, or emulsion.

In some examples, the compositions provided herein can be in the form of a capsule or tablet configured to have a unit dosage equal to the daily desired dosage for a particular subject (e.g., a human). For example, if a subject desires 100 mg of a particular active ingredient, each tablet can include about 100 mg in weight of that active ingredient. As used herein, subjects generally refer to mammals (e.g., humans), but also can include domesticated mammals (e.g., dogs, cats, and livestock, such as cows, horses, pigs, or sheep). The dosages of particular compositions provided herein will depend on many factors including the general health, age, and mass of the subject. In some examples, a total daily dose may be prepared and administered in the form of one or more dosage forms (e.g., two tablets or capsules, three tablets or capsules, four tablets or capsules, five tablets or capsules, or six tablets or capsules).

### Kits

Also provided herein are kits that include at least one dose of any of the compositions described herein. For example, provided herein are kits that include any of the capsules described herein. In some examples, an exemplary capsule can contain a therapeutically effective amount of a zeolite and a therapeutically effective amount of one or more additional active ingredients that include riboflavin, a colonic acidifier, an ammonia reducer, an ammonia sequestering agent, an intestinal deodorizer, and a probiotic.

### Subjects

In some embodiments of any of the methods described herein, the subject is a mammal (e.g., a human). In some embodiments of any of the methods described herein, the subject is in need of treatment, reduction, or alleviation of TMAU (e.g., primary or secondary TMAU). In some embodiments of any of the methods described herein, the subject has been diagnosed or previously diagnosed as having primary TMAU (TMAU1). In some embodiments of any of the methods described herein, the subject has been determined to have a dysregulating mutation in a flavin-containing monooxygenase 3 (FMO3) gene. In some embodiments, the subject has been previously diagnosed as having primary TMAU (TMAU1) (e.g., through genetic testing).

In some embodiments of any of the methods described herein, the subject has been diagnosed as having secondary TMAU (TMAU2). In some embodiments of any of the methods described herein, the subject does not have a urinary tract infection, bacterial vaginosis, cervical cancer, and advanced liver or kidney disease. In some embodiments of any of the methods described herein, the subject has been identified as having an enzymatic defect associated with secondary TMAU (TMAU2).

In some embodiments of any of the methods described herein, the subject has or has been identified as having an elevated level of trimethylamine his or her urine, sweat, reproductive fluid, saliva, blood, plasma, serum, or breath, as compared to a reference level of trimethylamine (e.g., a level of trimethylamine in the urine, sweat, reproductive fluid, saliva, blood, plasma, serum, or breath, respectively, of a subject not having TMAU (e.g., neither TMAU1 or TMAU2) or a subject not at genetic risk of developing TMAU1; or a threshold level of trimethylamine). Non-limiting examples of a threshold level of trimethylamine (in urine) can be, e.g., about 6.8 mg/L to about 11.0 mg/L, or 6.8 mg/L, 6.9 mg/L, 7.0 mg/L, 7.1 mg/L, 7.2 mg/L, 7.3 mg/L, 7.4 mg/L, 7.5 mg/L, 7.6 mg/L, 7.7 mg/L, 7.8 mg/L, 7.9 mg/L, 8.0 mg/L, 8.1 mg/L, 8.2 mg/L, 8.3 mg/L, 8.4 mg/L, 8.5 mg/L, 8.6 mg/L, 8.7 mg/L, 8.8 mg/L, 8.9 mg/L, 9.0 mg/L, 9.1 mg/L, 9.2 mg/L, 9.3 mg/L, 9.4 mg/L, 9.5 mg/L, 9.6 mg/L, 9.7 mg/L, 9.8 mg/L, 9.9 mg/L, 10.0 mg/L, 10.1 mg/L, 10.2 mg/L, 10.3 mg/L, 10.4 mg/L, 10.5 mg/L, 10.6 mg/L, 10.7 mg/L, 10.8 mg/L, 10.9 mg/L, or 11.0 mg/L.

In some embodiments of any of the methods described herein, the subject has or has been identified as having an elevated level of trimethylamine precursor in his or her urine, sweat, reproductive fluid, saliva, blood, plasma, serum, or breath, as compared to a reference level of trimethylamine precursor (e.g., a level of trimethylamine precursor in the urine, sweat, reproductive fluid, saliva, blood, plasma, serum, or breath, respectively, of a subject not having TMAU (e.g., neither TMAU1 or TMAU2) or a subject not at genetic risk of developing TMAU1; or a threshold level of trimethylamine precursor). Non-limiting examples of threshold level of a trimethylamine precursor can be defined by the upper limit of a level of a trimethylamine precursor in a healthy subject or a population of healthy subjects. Non-limiting examples of a trimethylamine precursor, as used anywhere herein, can be carnitine (total or free carnitine), choline, betaine, and trimethylamine-N-oxide. Exemplary threshold levels of total carnitine for an adult male (in serum) can be about 30 μmole/L to 70 μmole/L, or 30 μmole/L, 35 μmole/L, 40 μmole/L, 45 μmole/L, 50 μmole/L, 55 μmole/L, 60 μmole/L, 65 μmole/L, 70 μmole/L, 75 μmole/L, or 80 μmole/L. Exemplary threshold levels of total carnitine for an adult female (in serum) can be about 25 μmole/L to about 58 μmole/L, or 25 μmole/L, 30 μmole/L, 35 μmole/L, 40 μmole/L, 45 μmole/L, 50 μmole/L, 55 μmole/L, 60 μmole/L, or 65 μmole/L. Exemplary threshold levels of total carnitine for a child (age ≤17 years) (in serum) can be about 28 μmole/L to about 62 μmole/L, or 25 μmole/L, 30 μmole/L, 35 μmole/L, 40 mole/L, 45 μmole/L, 50 μmole/L, 55 μmole/L, about 60 μmole/L, or about 65 μmole/L. Exemplary threshold levels of free carnitine in an adult male (in serum) can be, e.g., about 23 μmole/L to about 59 μmole/L, or 35 μmole/L, 40 μmole/L, 45 μmole/L, 50 μmole/L, 55 μmole/L, 60 μmole/L, or 65 μmole/L. Exemplary threshold levels of free carnitine in adult women (in serum) can be, e.g., about 19 μmole/L to about 48 μmole/L, or 20 μmole/L, 25 μmole/L, 30 μmole/L, 35 μmole/L, 40 μmole/L, 45 μmole/L, 50 μmole/L, or 55 μmole/L. Exemplary threshold levels of free carnitine in children (age ≤17 years) (in serum) can be, e.g., about 19 μmole/L to about 54 μmole/L, or 20 μmole/L, 25 μmole/L, 30 μmole/L, 35 μmole/L, 40 μmole/L, 45 μmole/L, 50 μmole/L, 55 μmole/L, or 60 μmole/L. A threshold level of betaine (in plasma) can be, e.g., about 7.9 μM to about 12.2 μM, or 7.9 μM, 8.0 μM, 8.1 μM, 8.2 μM, 8.3 μM, 8.4 μM, 8.5 μM, 8.6 μM, 8.7 μM, 8.8 μM, 8.9 μM, 9.0 μM, 9.1 μM, 9.2 μM, 9.3 μM, 9.4 μM, 9.5 μM, 9.6 μM, 9.7 μM, 9.8 μM, 9.9 μM, 10.0 μM, 10.1 μM, 10.2 μM, 10.3 μM, 10.4 μM, 10.5 μM, 10.6 μM, 10.7 μM, 10.8 μM, 10.9 μM, 11.0 μM, 11.1 μM, 11.2 μM, 11.3 μM, 11.4 μM, 11.5 μM, 11.6 μM, 11.7 μM, 11.8 μM, 11.9 μM, 12.0 μM, 12.1 μM, 12.2 μM, 12.3 μM, 12.4 μM, or 12.5 μM. Non-limiting examples of threshold levels of choline (in plasma) can be, e.g., about 32.5 μM to about 52.1 μM, or 35 μM, 40 μM, 45 μM, 50 μM, or 55 μM. Non-limiting examples of threshold levels of trimethylamine-N-oxide (in plasma) can be, e.g., about 2.4 μM to about 6.2 μM, or 2.4 μM, 2.5 μM, 2.6 μM, 2.7 μM, 2.8 μM, 2.9 μM, 3.0 μM, 3.1 μM, 3.2 μM, 3.3 μM, 3.4 μM, 3.5 μM, 3.6 μM, 3.7 μM, 3.8 μM, 3.9 μM, 4.0 μM, 4.1 μM, 4.2 μM, 4.3 μM, 4.4 μM, 4.5 μM, 4.6 μM, 4.7 μM, 4.8 μM, 4.9 μM, 5.0 μM, 5.1 μM, 5.2 μM, 5.3 μM, 5.4 μM, 5.5 μM, 5.6 μM, 5.7 μM, 5.8 μM, 5.9 μM, 6.0 μM, 6.1 μM, 6.2 μM, 6.3 μM, 6.4 μM, or 6.5 μM. Additional examples of trimethylamine precursors and exemplary threshold values of exemplary trimethylamine precursors are described in, e.g., Wang et al., European Heart J. 35:904-910, 2014, and the Emedicine website (emedicine.medscape.com/article/2115721-overview).

In some embodiments of any of the methods described herein, the subject has or has been identified as having a decreased trimethylamine N-oxide ratio (as described herein) in his or her of urine, sweat, reproductive fluid, saliva, blood, plasma, serum, or breath, as compared to a reference level of trimethylamine N-oxide ratio (e.g., the trimethylamine N-oxide ratio in the urine, sweat, reproductive fluid, saliva, blood, plasma, serum, or breath, respectively, of a subject not having TMAU (e.g., neither TMAU1 or TMAU2) or a subject not at genetic risk of developing TMAU1; or a threshold trimethylamine N-oxide ratio). Non-limiting examples of a threshold trimethylamine N-oxide ratio include ratio values that are less than 20, e.g., 20, 19, 18, 17, 16, 15, 14, 13, 12, 11, 10, 9, 8, 7, 6, or 5.

In some embodiments of any of the methods described herein, the subject has been identified as having an elevated trimethylamine ratio (as described herein) in his or her of urine, sweat, reproductive fluid, saliva, blood, plasma, serum, or breath, as compared to a reference level of trimethylamine ratio (e.g., the trimethylamine ratio in the urine, sweat, reproductive fluid, saliva, blood, plasma, serum, or breath, respectively, of a subject not having TMAU (e.g., neither TMAU1 or TMAU2) or a subject not at genetic risk of developing TMAU1; or a threshold trimethylamine ratio). Non-limiting examples of threshold trimethylamine ratio values can be, e.g., greater than 80, e.g., 81, 82, 83, 84, 85, 86, 87, 88, 89, 90, 91, 92, 93, 94, or 95.

In some embodiments of any of the methods described herein, the subject is an adult. In some embodiments of any of the methods described herein, the subject is an adolescent. In some embodiments of any of the methods described herein, the subject is a child.

As used herein, “adults” are humans who are at least 18 years old, “non-elderly” are adult humans who are 18 to 64 years old, “elderly” are adult humans who are at least 65 years old, and “children” are humans who are 17 years old or younger.

In some embodiments of any of the methods described herein, the subject can be about 1 month old to about 100 years old, about 95 years old, about 90 years old, about 85 years old, about 80 years old, about 75 years old, about 70 years old, about 65 years old, about 60 years old, about 55 years old, about 50 years old, about 45 years old, about 40 years old, about 35 years old, about 30 years old, about 25 years old, about 21 years old, about 20 years old, about 19 years old, about 18 years old, about 10 years old, about 5 years old, about 4 years old, about 3 years old, about 2 years old, about 1 year old, or about 6 months old, inclusive; about 6 months old to about 100 years old, about 95 years old, about 90 years old, about 85 years old, about 80 years old, about 75 years old, about 70 years old, about 65 years old, about 60 years old, about 55 years old, about 50 years old, about 45 years old, about 40 years old, about 35 years old, about 30 years old, about 25 years old, about 21 years old, about 20 years old, about 19 years old, about 18 years old, about 10 years old, about 5 years old, about 4 years old, about 3 years old, about 2 years old, or about 1 year old, inclusive; about 1 year to about 100 years old, about 95 years old, about 90 years old, about 85 years old, about 80 years old, about 75 years old, about 70 years old, about 65 years old, about 60 years old, about 55 years old, about 50 years old, about 45 years old, about 40 years old, about 35 years old, about 30 years old, about 25 years old, about 21 years old, about 20 years old, about 19 years old, about 18 years old, about 10 years old, about 5 years old, about 4 years old, about 3 years old, or about 2 years old, inclusive; about 2 years to about 100 years old, about 95 years old, about 90 years old, about 85 years old, about 80 years old, about 75 years old, about 70 years old, about 65 years old, about 60 years old, about 55 years old, about 50 years old, about 45 years old, about 40 years old, about 35 years old, about 30 years old, about 25 years old, about 21 years old, about 20 years old, about 19 years old, about 18 years old, about 10 years old, about 5 years old, about 4 years old, or about 3 years old, inclusive; about 3 years to about 100 years old, about 95 years old, about 90 years old, about 85 years old, about 80 years old, about 75 years old, about 70 years old, about 65 years old, about 60 years old, about 55 years old, about 50 years old, about 45 years old, about 40 years old, about 35 years old, about 30 years old, about 25 years old, about 21 years old, about 20 years old, about 19 years old, about 18 years old, about 10 years old, about 5 years old, or about 4 years old, inclusive; about 4 years old to about 100 years old, about 95 years old, about 90 years old, about 85 years old, about 80 years old, about 75 years old, about 70 years old, about 65 years old, about 60 years old, about 55 years old, about 50 years old, about 45 years old, about 40 years old, about 35 years old, about 30 years old, about 25 years old, about 21 years old, about 20 years old, about 19 years old, about 18 years old, about 10 years old, or about 5 years old, inclusive; about 5 years old to about 100 years old, about 95 years old, about 90 years old, about 85 years old, about 80 years old, about 75 years old, about 70 years old, about 65 years old, about 60 years old, about 55 years old, about 50 years old, about 45 years old, about 40 years old, about 35 years old, about 30 years old, about 25 years old, about 21 years old, about 20 years old, about 19 years old, about 18 years old, or about 10 years old, inclusive; about 10 years old to about 100 years old, about 95 years old, about 90 years old, about 85 years old, about 80 years old, about 75 years old, about 70 years old, about 65 years old, about 60 years old, about 55 years old, about 50 years old, about 45 years old, about 40 years old, about 35 years old, about 30 years old, about 25 years old, about 21 years old, about 20 years old, about 19 years old, or about 18 years old, inclusive; about 18 years old to about 100 years old, about 95 years old, about 90 years old, about 85 years old, about 80 years old, about 75 years old, about 70 years old, about 65 years old, about 60 years old, about 55 years old, about 50 years old, about 45 years old, about 40 years old, about 35 years old, about 30 years old, about 25 years old, about 21 years old, or about 20 years old, inclusive; about 19 years old to about 100 years old, about 95 years old, about 90 years old, about 85 years old, about 80 years old, about 75 years old, about 70 years old, about 65 years old, about 60 years old, about 55 years old, about 50 years old, about 45 years old, about 40 years old, about 35 years old, about 30 years old, about 25 years old, or about 21 years old, inclusive; about 20 years old to about 100 years old, about 95 years old, about 90 years old, about 85 years old, about 80 years old, about 75 years old, about 70 years old, about 65 years old, about 60 years old, about 55 years old, about 50 years old, about 45 years old, about 40 years old, about 35 years old, about 30 years old, or about 25 years old, inclusive; about 21 years old to about 100 years old, about 95 years old, about 90 years old, about 85 years old, about 80 years old, about 75 years old, about 70 years old, about 65 years old, about 60 years old, about 55 years old, about 50 years old, about 45 years old, about 40 years old, about 35 years old, about 30 years old, or about 25 years old, inclusive; about 25 years old to about 100 years old, about 95 years old, about 90 years old, about 85 years old, about 80 years old, about 75 years old, about 70 years old, about 65 years old, about 60 years old, about 55 years old, about 50 years old, about 45 years old, about 40 years old, about 35 years old, or about 30 years old, inclusive; about 30 years old to about 100 years old, about 95 years old, about 90 years old, about 85 years old, about 80 years old, about 75 years old, about 70 years old, about 65 years old, about 60 years old, about 55 years old, about 50 years old, about 45 years old, about 40 years old, or about 35 years old, inclusive; about 35 years old to about 100 years old, about 95 years old, about 90 years old, about 85 years old, about 80 years old, about 75 years old, about 70 years old, about 65 years old, about 60 years old, about 55 years old, about 50 years old, about 45 years old, or about 40 years old, inclusive; about 40 years old to about 100 years old, about 95 years old, about 90 years old, about 85 years old, about 80 years old, about 75 years old, about 70 years old, about 65 years old, about 60 years old, about 55 years old, about 50 years old, or about 45 years old, inclusive; about 45 years old to about 100 years old, about 95 years old, about 90 years old, about 85 years old, about 80 years old, about 75 years old, about 70 years old, about 65 years old, about 60 years old, about 55 years old, or about 50 years old, inclusive; about 50 years old to about 100 years old, about 95 years old, about 90 years old, about 85 years old, about 80 years old, about 75 years old, about 70 years old, about 65 years old, about 60 years old, or about 55 years old, inclusive; about 55 years old to about 100 years old, about 95 years old, about 90 years old, about 85 years old, about 80 years old, about 75 years old, about 70 years old, about 65 years old, or about 60 years old, inclusive; about 60 years old to about 100 years old, about 95 years old, about 90 years old, about 85 years old, about 80 years old, about 75 years old, about 70 years old, or about 65 years old, inclusive; about 65 years old to about 100 years old, about 95 years old, about 90 years old, about 85 years old, about 80 years old, about 75 years old, or about 70 years old, inclusive; about 70 years old to about 100 years old, about 95 years old, about 90 years old, about 85 years old, about 80 years old, or about 75 years old, inclusive; about 75 years old to about 100 years old, about 95 years old, about 90 years old, about 85 years old, or about 80 years old, inclusive; about 80 years old to about 100 years old, about 95 years old, about 90 years old, or about 85 years old, inclusive; about 85 years old to about 100 years old, about 95 years old, about 90 years old, inclusive; about 90 years old to about 100 years old, or about 95 years old, inclusive; about 95 years old to about 100 years old, inclusive.

### Methods of Treating TMAU

Provided herein are methods of treating TMAU (e.g., TMAU1 or TMAU2) in a subject in need thereof that include administering (e.g., orally administering) to the subject an effective amount (e.g., a therapeutically effective amount) of any of the compositions described herein.

As used herein, “TMAU1” is an inherited form of TMAU that is caused by a mutation in the gene encoding enzyme flavin containing mono-oxygenase type 3 (FMO3), where the mutation causes a decrease in the activity of the encoded FMO3 as compared to the wildtype FMO3 enzyme. FMO3 is a microsomal NADPH-dependent cytochrome P450 enzyme that oxidizes trimethylamine in the liver. FMO3 metabolizes a large number of chemical compounds including drugs, dietary intermediates, industrial chemicals, and agricultural chemicals. Due to the low specificity of FMO3 and the large number of drugs detoxified, TMAU1 patients may suffer from adverse drug reactions (e.g., with codeine, tamoxifen, ketoconazole, nicotine, cimetidine, ranitidine, and/or phenothiazine). Subjects with TMAU1 may experience hypertension after the ingestion of red wine, cheese, and/or chocolate, which produces the neurotransmitter tyramine, another FMO3-dependent compound. Subjects having TMAU1 may also suffer from migraines associated with tyramine-containing foods.

As used herein, “TMAU2” is characterized by an abnormally increased level of trimethylamine in one or more body fluid in a subject, where the subject expresses a FMO3 enzyme having substantially the same activity as a wildtype FMO3 enzyme.

In some examples, a subject has previously been diagnosed or identified as having TMAU (e.g., TMAU1 and TMAU2 described herein or known in the art). Some embodiments of any of the methods described herein further include selecting a subject having TMAU (e.g., TMAU1 or TMAU2). Some embodiments of any of the methods described herein further include diagnosing a subject as having TMAU (e.g., TMAU1 or TMAU2). Some embodiments of any of the methods described herein further include selecting any of the compositions described herein for a subject having TMAU (e.g., TMAU1 or TMAU2) (or a subject previously identified or diagnosed as having TMAU (e.g., TMAU1 or TMAU2)). Methods for diagnosing TMAU (e.g., TMAU1 or TMAU2) are known in the art. For example, TMAU (e.g., TMAU1 or TMAU2) can be diagnosed by a physician by determining the level of trimethylamine, the level of trimethylamine precursor, the trimethylamine N-oxide ratio, and/or the trimethylamine ratio in one or more of urine, sweat, reproductive fluid, saliva, blood, plasma, serum, and breath of a subject. The diagnosis or identification of TMAU1 in a subject can further include, e.g., performing a genetic test to identify a mutation in a gene encoding FMO3, where the mutation results in the production of FMO3 that has decreased enzymatic activity as compared to a wildtype FMO3. Non-limiting methods for determining the levels of trimethylamine, trimethylamine precursor, and trimethylamine N-oxide include capillary electrophoresis, titration and colorimetric measurement, gas sensors, steam distillation, NMR spectroscopy, micro-diffusion, isotope dilution, antibody detection, and use of biosensors. See, e.g., the methods described in Li et al., J. Chromatography A 1216: 5924-5926 2009; Ocque et al., J. Pharm. Biomedical Analysis, 2015; Podadera et al., Clinica Chimica Acta 351:149-154, 2005; Suska et al., Biosensors Bioelectronics 25:715-720, 2009. Non-limiting methods for identifying a mutation in a gene encoding FMO3 that results in the production of FMO3 that has decreased enzymatic activity as compared to a wildtype FMO3 may include, e.g., multiplex ligation-dependent probe amplification (MLPA), single-strand conformation polymorphism (SSCP), denaturing gradient gel electrophoresis (DGGE), restriction fragment length polymorphism (RFLP), and microarray. See, e.g., the methods described in Mandieh et al., Iran J. Pediatr. 23(4): 375-388, 2013.

A “trimethylamine N-oxide ratio” is defined by Formula I:

[(level of trimethylamine N-oxide)/(level of trimethylamine N-oxide+level of trimethylamine)]×100%.

A “trimethylamine ratio” is defined by Formula II:

[(level of trimethylamine)/(level of trimethylamine N-oxide+level of trimethylamine)]×100%.

In some embodiments of any of the methods described herein, the methods of treating TMAU (e.g., TMAU1 or TMAU2) provided herein can result in one or more (e.g., two, three, or four) of: a decrease in the level of trimethylamine, a decrease in the level of trimethylamine precursor, an increase in trimethylamine N-oxide ratio, and/or a decrease in the trimethylamine ratio, in one or more of urine, sweat, reproductive fluid, saliva, blood, plasma, serum, and breath of a subject (e.g., as compared to the same one or more parameters in the subject prior to administration of any of the compositions described herein or in a subject or a population of subjects having the same TMAU but receiving a different treatment or a placebo).

For example, the methods of treating TMAU provided herein can result in an increase from about 1% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, about 90%, about 80%, about 70%, about 60%, about 50%, about 40%, about 30%, about 20%, about 10%, or about 5%, inclusive; about 5% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, about 90%, about 80%, about 70%, about 60%, about 50%, about 40%, about 30%, about 20%, or about 10%, inclusive; about 10% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, about 90%, about 80%, about 70%, about 60%, about 50%, about 40%, about 30%, or about 20%, inclusive; about 20% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, about 90%, about 80%, about 70%, about 60%, about 50%, about 40%, or about 30%, inclusive; about 30% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, about 90%, about 80%, about 70%, about 60%, about 50%, or about 40%, inclusive; about 40% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, about 90%, about 80%, about 70%, about 60%, or about 50%, inclusive; about 50% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, about 90%, about 80%, about 70%, or about 60%, inclusive; about 60% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, about 90%, about 80%, or about 70%, inclusive; about 70% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, about 90%, or about 80%, inclusive; about 80% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, or about 90%, inclusive; about 90% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, or about 100%, inclusive; about 100% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, inclusive; about 110% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, or about 120%, inclusive; about 120% to about 300%, about 290%, about 280%, about 270%, about 260%, about 250%, about 240%, about 230%, about 220%, about 210%, about 200%, about 190%, about 180%, about 170%, about 160%, about 150%, about 140%, or about 130%, inclusive; about 130% to about 300%, about 290%, about 280%, about 270%, about 260%, about 250%, about 240%, about 230%, about 220%, about 210%, about 200%, about 190%, about 180%, about 170%, about 160%, about 150%, or about 140%, inclusive; about 140% to about 300%, about 290%, about 280%, about 270%, about 260%, about 250%, about 240%, about 230%, about 220%, about 210%, about 200%, about 190%, about 180%, about 170%, about 160%, or about 150%, inclusive; about 150% to about 300%, about 290%, about 280%, about 270%, about 260%, about 250%, about 240%, about 230%, about 220%, about 210%, about 200%, about 190%, about 180%, about 170%, or about 160%, inclusive; about 160% to about 300%, about 290%, about 280%, about 270%, about 260%, about 250%, about 240%, about 230%, about 220%, about 210%, about 200%, about 190%, about 180%, or about 170%, inclusive; about 170% to about 300%, about 290%, about 280%, about 270%, about 260%, about 250%, about 240%, about 230%, about 220%, about 210%, about 200%, about 190%, or about 180%, inclusive; about 180% to about 300%, about 290%, about 280%, about 270%, about 260%, about 250%, about 240%, about 230%, about 220%, about 210%, about 200%, or about 190%, inclusive; about 190% to about 300%, about 290%, about 280%, about 270%, about 260%, about 250%, about 240%, about 230%, about 220%, about 210%, or about 200%, inclusive; about 200% to about 300%, about 290%, about 280%, about 270%, about 260%, about 250%, about 240%, about 230%, about 220%, or about 210%, inclusive; about 210% to about 300%, about 290%, about 280%, about 270%, about 260%, about 250%, about 240%, about 230%, or about 220%, inclusive; about 220% to about 300%, about 290%, about 280%, about 270%, about 260%, about 250%, about 240%, or about 230%, inclusive; about 230% to about 300%, about 290%, about 280%, about 270%, about 260%, about 250%, or about 240%, inclusive; about 240% to about 300%, about 290%, about 280%, about 270%, about 260%, about 250%, inclusive; about 250% to about 300%, about 290%, about 280%, about 270%, about 260%, inclusive; about 260% to about 300%, about 290%, about 280%, or about 270%, inclusive; about 270% to about 300%, about 290%, or about 280%, inclusive; about 280% to about 300%, or about 290%, inclusive; about 290% to about 300%, inclusive, in the trimethylamine N-oxide ratio in one or more of urine, sweat, reproductive fluid, saliva, blood, plasma, serum, and breath in a subject (e.g., as compared to the same one or more parameters in the subject prior to administration of any of the compositions described herein or in a subject or a population of subjects having the same TMAU but receiving a different treatment or a placebo).

For example, the methods of treating TMAU provided herein can result in a decrease in one or more (e.g., two, or three) of the level of trimethylamine, the level of trimethylamine precursor, and/or the trimethylamine ratio, in one or more of urine, sweat, reproductive fluid, saliva, blood, plasma, serum, and breath of a subject to a level(s) that fall(s) within a normal range for a subject or a population of subjects not having TMAU (or a subject not presenting with one or more symptoms of TMAU).

In some examples, the methods of treating TMAU provided herein can result in a decrease from about 1% to about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%, inclusive; about 5% to about 90%, about 85%, about 80%, about 75%, about 70%, about 65%, about 60%, about 55%, about 50%, about 45%, about 40%, about 35%, about 30%, about 25%, about 20%, about 15%, or about 10%, inclusive; about 10% to about 90%, about 85%, about 80%, about 75%, about 70%, about 65%, about 60%, about 55%, about 50%, about 45%, about 40%, about 35%, about 30%, about 25%, about 20%, or about 15%, inclusive; about 15% to about 90%, about 85%, about 80%, about 75%, about 70%, about 65%, about 60%, about 55%, about 50%, about 45%, about 40%, about 35%, about 30%, about 25%, or about 20%, inclusive; about 20% to about 90%, about 85%, about 80%, about 75%, about 70%, about 65%, about 60%, about 55%, about 50%, about 45%, about 40%, about 35%, about 30%, or about 25%, inclusive; about 25% to about 90%, about 85%, about 80%, about 75%, about 70%, about 65%, about 60%, about 55%, about 50%, about 45%, about 40%, about 35%, or about 30%, inclusive; about 30% to about 90%, about 85%, about 80%, about 75%, about 70%, about 65%, about 60%, about 55%, about 50%, about 45%, about 40%, or about 35%, inclusive; about 35% to about 90%, about 85%, about 80%, about 75%, about 70%, about 65%, about 60%, about 55%, about 50%, about 45%, or about 40%, inclusive; about 40% to about 90%, about 85%, about 80%, about 75%, about 70%, about 65%, about 60%, about 55%, about 50%, about 45%, inclusive; about 45% to about 90%, about 85%, about 80%, about 75%, about 70%, about 65%, about 60%, about 55%, or about 50%, inclusive; about 50% to about 90%, about 85%, about 80%, about 75%, about 70%, about 65%, about 60%, or about 55%, inclusive; about 55% to about 90%, about 85%, about 80%, about 75%, about 70%, about 65%, or about 60%, inclusive; about 60% to about 90%, about 85%, about 80%, about 75%, about 70%, about 65%, inclusive; about 65% to about 90%, about 85%, about 80%, about 75%, or about 70%, inclusive; about 70% to about 90%, about 85%, about 80%, or about 75%, inclusive; about 75% to about 90%, about 85%, or about 80%, inclusive; about 80% to about 90%, or about 85%, inclusive; or about 85% to about 90%, inclusive, in one or more (e.g., two or three) of the level of trimethylamine, the level of trimethylamine precursor, and the trimethylamine ratio in one or more of urine, sweat, reproductive fluid, saliva, blood, plasma, serum, and breath of a subject (e.g., as compared to the same one or more parameters in the subject prior to administration of any of the compositions described herein or in a subject or a population of subjects having the same TMAU but receiving a different treatment or a placebo).

In some embodiments, the methods of treating TMAU provided herein can result in an increase in the subject's satisfaction with his or her body odor (e.g., as compared to the subject's satisfaction prior to administration of any of the compositions described herein). Methods of determining a subject's satisfaction with his or her body odor can be determined using, e.g., a patient survey.

In some examples, a subject can be administered (e.g., orally administered) any of the compositions described herein once a day, twice a day, three times a day, four times a day, every other day, every third day, every fourth day, every fifth day, every sixth day, or once a week. In some embodiments, the subject is started at an initial dose amount and the dosage can be subsequently changed (e.g., decreased or increased). A dose or unit dose can be a fraction of the daily dose, such as the daily dose divided by the number of doses or unit doses to be administered per day. The timing of the administration of the compositions provided herein will depend on the formulation and/or route of administration used.

In some examples of any of the methods described herein, a subject is administered a composition including a zeolite (e.g., any of the compositions described herein) about 1 minute to about 5 hours, about 4.5 hours, about 4 hours, about 3.5 hours, about 3 hours, about 2.5 hours, about 2 hours, about 1.5 hours, about 1 hour, about 50 minutes, about 40 minutes, about 30 minutes, about 25 minutes, about 20 minutes, about 15 minutes, about 10 minutes, or about 5 minutes, inclusive, about 5 minutes to about 5 hours, about 4.5 hours, about 4 hours, about 3.5 hours, about 3 hours, about 2.5 hours, about 2 hours, about 1.5 hours, about 1 hour, about 50 minutes, about 40 minutes, about 30 minutes, about 25 minutes, about 20 minutes, about 15 minutes, or about 10 minutes, inclusive; about 10 minutes to about 5 hours, about 4.5 hours, about 4 hours, about 3.5 hours, about 3 hours, about 2.5 hours, about 2 hours, about 1.5 hours, about 1 hour, about 50 minutes, about 40 minutes, about 30 minutes, about 25 minutes, about 20 minutes, or about 15 minutes, inclusive; about 15 minutes to about 5 hours, about 4.5 hours, about 4 hours, about 3.5 hours, about 3 hours, about 2.5 hours, about 2 hours, about 1.5 hours, about 1 hour, about 50 minutes, about 40 minutes, about 30 minutes, about 25 minutes, or about 20 minutes, inclusive; about 20 minutes to about 5 hours, about 4.5 hours, about 4 hours, about 3.5 hours, about 3 hours, about 2.5 hours, about 2 hours, about 1.5 hours, about 1 hour, about 50 minutes, about 40 minutes, about 30 minutes, or about 25 minutes, inclusive; about 25 minutes to about 5 hours, about 4.5 hours, about 4 hours, about 3.5 hours, about 3 hours, about 2.5 hours, about 2 hours, about 1.5 hours, about 1 hour, about 50 minutes, about 40 minutes, or about 30 minutes, inclusive; about 30 minutes to about 5 hours, about 4.5 hours, about 4 hours, about 3.5 hours, about 3 hours, about 2.5 hours, about 2 hours, about 1.5 hours, about 1 hour, about 50 minutes, or about 40 minutes, inclusive; about 40 minutes to about 5 hours, about 4.5 hours, about 4 hours, about 3.5 hours, about 3 hours, about 2.5 hours, about 2 hours, about 1.5 hours, about 1 hour, about 50 minutes, inclusive; about 50 minutes to about 5 hours, about 4.5 hours, about 4 hours, about 3.5 hours, about 3 hours, about 2.5 hours, about 2 hours, about 1.5 hours, about 1 hour, inclusive; about 1 hour to about 5 hours, about 4.5 hours, about 4 hours, about 3.5 hours, about 3 hours, about 2.5 hours, about 2 hours, or about 1.5 hours, inclusive; about 1.5 hours to about 5 hours, about 4.5 hours, about 4 hours, about 3.5 hours, about 3 hours, about 2.5 hours, or about 2 hours, inclusive; about 2 hours to about 5 hours, about 4.5 hours, about 4 hours, about 3.5 hours, about 3 hours, or about 2.5 hours, inclusive; about 2.5 hours to about 5 hours, about 4.5 hours, about 4 hours, about 3.5 hours, or about 3 hours, inclusive; about 3 hours to about 5 hours, about 4.5 hours, about 4 hours, or about 3.5 hours, inclusive; about 3.5 hours to about 5 hours, about 4.5 hours, or about 4 hours, inclusive; about 4 hours to about 5 hours, or about 4.5 hours, inclusive; about 4.5 hours to about 5 hours, inclusive, before or after the subject has eaten a meal.

In some examples, the subject can be treated over a total period of time of about 1 week to about 90 years, about 80 years, about 70 years, about 60 years, about 50 years, about 40 years, about 30 years, about 20 years, about 10 years, about 5 years, about 2 years, about 1 year, about 6 months, about 2 months, or about 1 month, inclusive.

The composition including a zeolite administered in any of the methods described herein can be any of the compositions described herein. In some embodiments of any of the methods described herein, the subject can be any of the subjects described herein. In some embodiments of any of the methods described herein, the subject can be administered any of the doses of any of the compositions described herein.

Methods of Decreasing the Level of Trimethylamine, Decreasing the Level of Trimethylamine Precursor, Increasing the Level of Trimethylamine N-Oxide Ratio, and/or Decreasing the Trimethylamine Ratio in a Subject in Need Thereof

Also provided herein are methods of decreasing a level of trimethylamine in one or more of urine, sweat, reproductive fluid, saliva, blood, plasma, serum, and breath in a subject in need thereof (e.g., any of the subjects described herein) that include administering (e.g., orally administering) an amount (e.g., a therapeutically effective amount) of any of the composition including a zeolite described herein. Some examples of these methods can result in a decrease from about 1% to about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%, inclusive; about 5% to about 90%, about 85%, about 80%, about 75%, about 70%, about 65%, about 60%, about 55%, about 50%, about 45%, about 40%, about 35%, about 30%, about 25%, about 20%, about 15%, or about 10%, inclusive; about 10% to about 90%, about 85%, about 80%, about 75%, about 70%, about 65%, about 60%, about 55%, about 50%, about 45%, about 40%, about 35%, about 30%, about 25%, about 20%, or about 15%, inclusive; about 15% to about 90%, about 85%, about 80%, about 75%, about 70%, about 65%, about 60%, about 55%, about 50%, about 45%, about 40%, about 35%, about 30%, about 25%, or about 20%, inclusive; about 20% to about 90%, about 85%, about 80%, about 75%, about 70%, about 65%, about 60%, about 55%, about 50%, about 45%, about 40%, about 35%, about 30%, or about 25%, inclusive; about 25% to about 90%, about 85%, about 80%, about 75%, about 70%, about 65%, about 60%, about 55%, about 50%, about 45%, about 40%, about 35%, or about 30%, inclusive; about 30% to about 90%, about 85%, about 80%, about 75%, about 70%, about 65%, about 60%, about 55%, about 50%, about 45%, about 40%, or about 35%, inclusive; about 35% to about 90%, about 85%, about 80%, about 75%, about 70%, about 65%, about 60%, about 55%, about 50%, about 45%, or about 40%, inclusive; about 40% to about 90%, about 85%, about 80%, about 75%, about 70%, about 65%, about 60%, about 55%, about 50%, about 45%, inclusive; about 45% to about 90%, about 85%, about 80%, about 75%, about 70%, about 65%, about 60%, about 55%, or about 50%, inclusive; about 50% to about 90%, about 85%, about 80%, about 75%, about 70%, about 65%, about 60%, or about 55%, inclusive; about 55% to about 90%, about 85%, about 80%, about 75%, about 70%, about 65%, or about 60%, inclusive; about 60% to about 90%, about 85%, about 80%, about 75%, about 70%, about 65%, inclusive; about 65% to about 90%, about 85%, about 80%, about 75%, or about 70%, inclusive; about 70% to about 90%, about 85%, about 80%, or about 75%, inclusive; about 75% to about 90%, about 85%, or about 80%, inclusive; about 80% to about 90%, or about 85%, inclusive; or about 85% to about 90%, inclusive, in the level of trimethylamine in one or more of urine, sweat, reproductive fluid, saliva, blood, plasma, serum, and breath of a subject (e.g., as compared to the same parameter in the subject prior to administration of any of the compositions described herein or in a subject or a population of subjects having the same TMAU but receiving a different treatment or a placebo). Some embodiments of these methods can result in a decrease in the level of trimethylamine in one or more of urine, sweat, reproductive fluid, saliva, blood, plasma, serum, and breath in a subject in need thereof (e.g., any of the subjects described herein) to a level that falls within a normal range for a subject or a population of subjects not having a TMAU (or a subject or a population of subjects not presenting with one or more symptoms of a TMAU) (e.g., less than 11.0 mg/L or less than 10.0 mg/L (in urine), or between about 6.8 mg/L to about 11.0 mg/L (in urine)). In some embodiments of these methods, the subject has been previously identified or diagnosed as having an elevated level of trimethylamine in one or more of urine, sweat, reproductive fluid, saliva, blood, plasma, serum, and breath (e.g., as compared to a normal range for a subject or a population of subjects not having a TMAU (or a subject or population of subjects not presenting with one or more symptoms of a TMAU). In some embodiments, the subject has been previously diagnosed or identified as having TMAU (e.g., TMAU1 or TMAU2).

Also provided herein are methods of decreasing a level of trimethylamine precursor in one or more of urine, sweat, reproductive fluid, saliva, blood, plasma, serum, and breath in a subject in need thereof (e.g., any of the subjects described herein) that include administering (e.g., orally administering) an amount (e.g., a therapeutically effective amount) of any of the composition including a zeolite described herein. Some examples of these methods can result in a decrease from about 1% to about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%, inclusive; about 5% to about 90%, about 85%, about 80%, about 75%, about 70%, about 65%, about 60%, about 55%, about 50%, about 45%, about 40%, about 35%, about 30%, about 25%, about 20%, about 15%, or about 10%, inclusive; about 10% to about 90%, about 85%, about 80%, about 75%, about 70%, about 65%, about 60%, about 55%, about 50%, about 45%, about 40%, about 35%, about 30%, about 25%, about 20%, or about 15%, inclusive; about 15% to about 90%, about 85%, about 80%, about 75%, about 70%, about 65%, about 60%, about 55%, about 50%, about 45%, about 40%, about 35%, about 30%, about 25%, or about 20%, inclusive; about 20% to about 90%, about 85%, about 80%, about 75%, about 70%, about 65%, about 60%, about 55%, about 50%, about 45%, about 40%, about 35%, about 30%, or about 25%, inclusive; about 25% to about 90%, about 85%, about 80%, about 75%, about 70%, about 65%, about 60%, about 55%, about 50%, about 45%, about 40%, about 35%, or about 30%, inclusive; about 30% to about 90%, about 85%, about 80%, about 75%, about 70%, about 65%, about 60%, about 55%, about 50%, about 45%, about 40%, or about 35%, inclusive; about 35% to about 90%, about 85%, about 80%, about 75%, about 70%, about 65%, about 60%, about 55%, about 50%, about 45%, or about 40%, inclusive; about 40% to about 90%, about 85%, about 80%, about 75%, about 70%, about 65%, about 60%, about 55%, about 50%, about 45%, inclusive; about 45% to about 90%, about 85%, about 80%, about 75%, about 70%, about 65%, about 60%, about 55%, or about 50%, inclusive; about 50% to about 90%, about 85%, about 80%, about 75%, about 70%, about 65%, about 60%, or about 55%, inclusive; about 55% to about 90%, about 85%, about 80%, about 75%, about 70%, about 65%, or about 60%, inclusive; about 60% to about 90%, about 85%, about 80%, about 75%, about 70%, about 65%, inclusive; about 65% to about 90%, about 85%, about 80%, about 75%, or about 70%, inclusive; about 70% to about 90%, about 85%, about 80%, or about 75%, inclusive; about 75% to about 90%, about 85%, or about 80%, inclusive; about 80% to about 90%, or about 85%, inclusive; or about 85% to about 90%, inclusive, in the level of trimethylamine precursor in one or more of urine, sweat, reproductive fluid, saliva, blood, plasma, serum, and breath of a subject (e.g., as compared to the same parameter in the subject prior to administration of any of the compositions described herein or in a subject or a population of subjects having the same TMAU but receiving a different treatment or a placebo). Some embodiments of these methods can result in a decrease in a level of trimethylamine precursor in one or more of urine, sweat, reproductive fluid, saliva, blood, plasma, serum, and breath to a level that falls within a normal range for a subject or a population of subjects not having a TMAU (or a subject or a population of subjects not presenting with one or more symptoms of a TMAU). An exemplary normal range of total carnitine (in serum) for an adult male or a population of adult males not having TMAU can be about 30 μmole/L to about 70 μmole/L. An exemplary normal range of total carnitine (in serum) for an adult female or a population of adult females not having TMAU can be about 25 μmole/L to about 58 μmole/L. An exemplary normal range of total carnitine (in serum) for a child or a population of children (age ≤17 years) not having a TMAU can be about 28 μmole/L to about 62 μmole/L. An exemplary normal range of free carnitine (in serum) in an adult male or a population of adult men not having a TMAU can be, e.g., about 23 μmole/L to about 59 μmole/L. An exemplary normal range of free carnitine in adult women (in serum) not having a TMAU can be, e.g., about 19 μmole/L to about 48 μmole/L. An exemplary normal range of free carnitine (in serum) in a child or a population of children not having a TMAU can be, e.g., about 19 μmole/L to about 54 μmole/L. An exemplary normal range of betaine (in plasma) in a subject or a population of subjects not having a TMAU can be, e.g., about 7.9 μM to about 12.2 μM. An exemplary normal range of choline (in plasma) in a subject or a population of subjects not having a TMAU can be, e.g., about 32.5 μM to about 52.1 μM.

In some embodiments of these methods, the subject has been previously identified or diagnosed as having an elevated level of trimethylamine precursor in one or more of urine, sweat, reproductive fluid, saliva, blood, plasma, serum, and breath (e.g., as compared to a normal range for a subject or a population of subjects not having a TMAU (or a subject or population of subjects not presenting with one or more symptoms of a TMAU). In some embodiments, the subject has been previously diagnosed or identified as having TMAU (e.g., TMAU1 or TMAU2).

Also provided herein are methods of increasing a trimethylamine N-oxide ratio in one or more of urine, sweat, reproductive fluid, saliva, blood, plasma, serum, and breath in a subject in need thereof (e.g., any of the subjects described herein) that include administering (e.g., orally administering) an amount (e.g., a therapeutically effective amount) of any of the composition including a zeolite described herein. Some examples of these methods can result in an increase from about 1% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, about 90%, about 80%, about 70%, about 60%, about 50%, about 40%, about 30%, about 20%, about 10%, or about 5%, inclusive; about 5% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, about 90%, about 80%, about 70%, about 60%, about 50%, about 40%, about 30%, about 20%, or about 10%, inclusive; about 10% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, about 90%, about 80%, about 70%, about 60%, about 50%, about 40%, about 30%, or about 20%, inclusive; about 20% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, about 90%, about 80%, about 70%, about 60%, about 50%, about 40%, or about 30%, inclusive; about 30% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, about 90%, about 80%, about 70%, about 60%, about 50%, or about 40%, inclusive; about 40% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, about 90%, about 80%, about 70%, about 60%, or about 50%, inclusive; about 50% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, about 90%, about 80%, about 70%, or about 60%, inclusive; about 60% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, about 90%, about 80%, or about 70%, inclusive; about 70% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, about 90%, or about 80%, inclusive; about 80% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, about 100%, or about 90%, inclusive; about 90% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, or about 100%, inclusive; about 100% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, about 120%, about 110%, inclusive; about 110% to about 300%, about 290%, about 280%, about 270%, about 260%, about 250%, about 240%, about 230%, about 220%, about 210%, about 200%, about 190%, about 180%, about 170%, about 160%, about 150%, about 140%, about 130%, or about 120%, inclusive; about 120% to about 300%, about 290%, about 280%, about 270%, about 260%, about 250%, about 240%, about 230%, about 220%, about 210%, about 200%, about 190%, about 180%, about 170%, about 160%, about 150%, about 140%, or about 130%, inclusive; about 130% to about 300%, about 290%, about 280%, about 270%, about 260%, about 250%, about 240%, about 230%, about 220%, about 210%, about 200%, about 190%, about 180%, about 170%, about 160%, about 150%, or about 140%, inclusive; about 140% to about 300%, about 290%, about 280%, about 270%, about 260%, about 250%, about 240%, about 230%, about 220%, about 210%, about 200%, about 190%, about 180%, about 170%, about 160%, or about 150%, inclusive; about 150% to about 300%, about 290%, about 280%, about 270%, about 260%, about 250%, about 240%, about 230%, about 220%, about 210%, about 200%, about 190%, about 180%, about 170%, or about 160%, inclusive; about 160% to about 300%, about 290%, about 280%, about 270%, about 260%, about 250%, about 240%, about 230%, about 220%, about 210%, about 200%, about 190%, about 180%, or about 170%, inclusive; about 170% to about 300%, about 290%, about 280%, about 270%, about 260%, about 250%, about 240%, about 230%, about 220%, about 210%, about 200%, about 190%, or about 180%, inclusive; about 180% to about 300%, about 290%, about 280%, about 270%, about 260%, about 250%, about 240%, about 230%, about 220%, about 210%, about 200%, or about 190%, inclusive; about 190% to about 300%, about 290%, about 280%, about 270%, about 260%, about 250%, about 240%, about 230%, about 220%, about 210%, or about 200%, inclusive; about 200% to about 300%, about 290%, about 280%, about 270%, about 260%, about 250%, about 240%, about 230%, about 220%, or about 210%, inclusive; about 210% to about 300%, about 290%, about 280%, about 270%, about 260%, about 250%, about 240%, about 230%, or about 220%, inclusive; about 220% to about 300%, about 290%, about 280%, about 270%, about 260%, about 250%, about 240%, or about 230%, inclusive; about 230% to about 300%, about 290%, about 280%, about 270%, about 260%, about 250%, or about 240%, inclusive; about 240% to about 300%, about 290%, about 280%, about 270%, about 260%, about 250%, inclusive; about 250% to about 300%, about 290%, about 280%, about 270%, about 260%, inclusive; about 260% to about 300%, about 290%, about 280%, or about 270%, inclusive; about 270% to about 300%, about 290%, or about 280%, inclusive; about 280% to about 300%, or about 290%, inclusive; about 290% to about 300%, inclusive, in the trimethylamine N-oxide ratio in the urine, sweat, reproductive fluid, saliva, blood, plasma, serum, and breath in a subject (e.g., as compared to the same parameter in the subject prior to administration of any of the compositions described herein or in a subject or a population of subjects having the same TMAU but receiving a different treatment or a placebo). Some embodiments of these methods can result in an increase in the trimethylamine N-oxide ratio in one or more of the urine, sweat, reproductive fluid, saliva, blood, plasma, serum, and breath from the subject to a level that falls within a normal range for a subject or a population of subjects not having a TMAU (or a subject or a population of subjects not presenting with one or more symptoms of a TMAU). In some embodiments of these methods, the subject has been previously identified or diagnosed as having a decreased trimethylamine N-oxide ratio in one or more of urine, sweat, reproductive fluid, saliva, blood, plasma, serum, and breath (e.g., as compared to a normal range for a subject or a population of subjects not having a TMAU (or a subject or population of subjects not presenting with one or more symptoms of a TMAU). In some embodiments, the subject has been previously diagnosed or identified as having a TMAU (e.g., TMAU1 or TMAU2).

Also provided herein are methods of decreasing a trimethylamine ratio in one or more of urine, sweat, reproductive fluid, saliva, blood, plasma, serum, and breath in a subject in need thereof (e.g., any of the subjects described herein) that include administering (e.g., orally administering) an amount (e.g., a therapeutically effective amount) of any of the composition including a zeolite described herein. Some examples of these methods can result in a decrease from about 1% to about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%, inclusive; about 5% to about 90%, about 85%, about 80%, about 75%, about 70%, about 65%, about 60%, about 55%, about 50%, about 45%, about 40%, about 35%, about 30%, about 25%, about 20%, about 15%, or about 10%, inclusive; about 10% to about 90%, about 85%, about 80%, about 75%, about 70%, about 65%, about 60%, about 55%, about 50%, about 45%, about 40%, about 35%, about 30%, about 25%, about 20%, or about 15%, inclusive; about 15% to about 90%, about 85%, about 80%, about 75%, about 70%, about 65%, about 60%, about 55%, about 50%, about 45%, about 40%, about 35%, about 30%, about 25%, or about 20%, inclusive; about 20% to about 90%, about 85%, about 80%, about 75%, about 70%, about 65%, about 60%, about 55%, about 50%, about 45%, about 40%, about 35%, about 30%, or about 25%, inclusive; about 25% to about 90%, about 85%, about 80%, about 75%, about 70%, about 65%, about 60%, about 55%, about 50%, about 45%, about 40%, about 35%, or about 30%, inclusive; about 30% to about 90%, about 85%, about 80%, about 75%, about 70%, about 65%, about 60%, about 55%, about 50%, about 45%, about 40%, or about 35%, inclusive; about 35% to about 90%, about 85%, about 80%, about 75%, about 70%, about 65%, about 60%, about 55%, about 50%, about 45%, or about 40%, inclusive; about 40% to about 90%, about 85%, about 80%, about 75%, about 70%, about 65%, about 60%, about 55%, about 50%, about 45%, inclusive; about 45% to about 90%, about 85%, about 80%, about 75%, about 70%, about 65%, about 60%, about 55%, or about 50%, inclusive; about 50% to about 90%, about 85%, about 80%, about 75%, about 70%, about 65%, about 60%, or about 55%, inclusive; about 55% to about 90%, about 85%, about 80%, about 75%, about 70%, about 65%, or about 60%, inclusive; about 60% to about 90%, about 85%, about 80%, about 75%, about 70%, about 65%, inclusive; about 65% to about 90%, about 85%, about 80%, about 75%, or about 70%, inclusive; about 70% to about 90%, about 85%, about 80%, or about 75%, inclusive; about 75% to about 90%, about 85%, or about 80%, inclusive; about 80% to about 90%, or about 85%, inclusive; or about 85% to about 90%, inclusive, in the trimethylamine ratio in one or more of urine, sweat, reproductive fluid, saliva, blood, plasma, serum, and breath in a subject (e.g., as compared to the same parameter in the subject prior to administration of any of the compositions described herein or in a subject or a population of subjects having the same TMAU but receiving a different treatment or a placebo). Some embodiments of these methods can result in a decrease in the trimethylamine ratio in one or more of urine, sweat, reproductive fluid, saliva, blood, plasma, serum, and breath in the subject to a level that falls within a normal range for a subject or a population of subjects not having a TMAU (or a subject or a population of subjects not presenting with one or more symptoms of a TMAU). In some embodiments of these methods, the subject has been previously identified or diagnosed as having an elevated trimethylamine ratio in one or more of urine, sweat, reproductive fluid, saliva, blood, plasma, serum, and breath (e.g., as compared to a normal range for a subject or a population of subjects not having a TMAU (or a subject or population of subjects not presenting with one or more symptoms of a TMAU). In some embodiments, the subject has been previously diagnosed or identified as having a TMAU (e.g., TMAU1 or TMAU2).

In some examples of any of the methods provided herein, a subject can be administered (e.g., orally administered) any of the compositions described herein once a day, twice a day, three times a day, four times a day, every other day, every third day, every fourth day, every fifth day, every sixth day, or once a week. In some embodiments, the subject is started at an initial dose amount and the dosage can be subsequently changed (e.g., decreased or increased). A dose or unit dose can be a fraction of the daily dose, such as the daily dose divided by the number of doses or unit doses to be administered per day. The timing of the administration of the compositions provided herein will depend on the formulation and/or route of administration used. In some examples of any of the methods described herein, a subject is administered a composition including a zeolite about 1 minute to about 5 hours, about 4.5 hours, about 4 hours, about 3.5 hours, about 3 hours, about 2.5 hours, about 2 hours, about 1.5 hours, about 1 hour, about 50 minutes, about 40 minutes, about 30 minutes, about 25 minutes, about 20 minutes, about 15 minutes, about 10 minutes, or about 5 minutes, inclusive, about 5 minutes to about 5 hours, about 4.5 hours, about 4 hours, about 3.5 hours, about 3 hours, about 2.5 hours, about 2 hours, about 1.5 hours, about 1 hour, about 50 minutes, about 40 minutes, about 30 minutes, about 25 minutes, about 20 minutes, about 15 minutes, or about 10 minutes, inclusive; about 10 minutes to about 5 hours, about 4.5 hours, about 4 hours, about 3.5 hours, about 3 hours, about 2.5 hours, about 2 hours, about 1.5 hours, about 1 hour, about 50 minutes, about 40 minutes, about 30 minutes, about 25 minutes, about 20 minutes, or about 15 minutes, inclusive; about 15 minutes to about 5 hours, about 4.5 hours, about 4 hours, about 3.5 hours, about 3 hours, about 2.5 hours, about 2 hours, about 1.5 hours, about 1 hour, about 50 minutes, about 40 minutes, about 30 minutes, about 25 minutes, or about 20 minutes, inclusive; about 20 minutes to about 5 hours, about 4.5 hours, about 4 hours, about 3.5 hours, about 3 hours, about 2.5 hours, about 2 hours, about 1.5 hours, about 1 hour, about 50 minutes, about 40 minutes, about 30 minutes, or about 25 minutes, inclusive; about 25 minutes to about 5 hours, about 4.5 hours, about 4 hours, about 3.5 hours, about 3 hours, about 2.5 hours, about 2 hours, about 1.5 hours, about 1 hour, about 50 minutes, about 40 minutes, or about 30 minutes, inclusive; about 30 minutes to about 5 hours, about 4.5 hours, about 4 hours, about 3.5 hours, about 3 hours, about 2.5 hours, about 2 hours, about 1.5 hours, about 1 hour, about 50 minutes, or about 40 minutes, inclusive; about 40 minutes to about 5 hours, about 4.5 hours, about 4 hours, about 3.5 hours, about 3 hours, about 2.5 hours, about 2 hours, about 1.5 hours, about 1 hour, about 50 minutes, inclusive; about 50 minutes to about 5 hours, about 4.5 hours, about 4 hours, about 3.5 hours, about 3 hours, about 2.5 hours, about 2 hours, about 1.5 hours, about 1 hour, inclusive; about 1 hour to about 5 hours, about 4.5 hours, about 4 hours, about 3.5 hours, about 3 hours, about 2.5 hours, about 2 hours, or about 1.5 hours, inclusive; about 1.5 hours to about 5 hours, about 4.5 hours, about 4 hours, about 3.5 hours, about 3 hours, about 2.5 hours, or about 2 hours, inclusive; about 2 hours to about 5 hours, about 4.5 hours, about 4 hours, about 3.5 hours, about 3 hours, or about 2.5 hours, inclusive; about 2.5 hours to about 5 hours, about 4.5 hours, about 4 hours, about 3.5 hours, or about 3 hours, inclusive; about 3 hours to about 5 hours, about 4.5 hours, about 4 hours, or about 3.5 hours, inclusive; about 3.5 hours to about 5 hours, about 4.5 hours, or about 4 hours, inclusive; about 4 hours to about 5 hours, or about 4.5 hours, inclusive; about 4.5 hours to about 5 hours, inclusive, after or before the subject consumes a meal.

In some examples of any of the methods described herein, the subject can be treated over a total period of time of about 1 week to about 90 years, about 80 years, about 70 years, about 60 years, about 50 years, about 40 years, about 30 years, about 20 years, about 10 years, about 5 years, about 2 years, about 1 year, about 6 months, about 2 months, or about 1 month, inclusive.

The composition including a zeolite administered in any of the methods described herein can be any of the compositions described herein. In some embodiments of any of the methods described herein, the subject can be any of the subjects described herein. In some embodiments of any of the methods described herein, the subject can be administered any of the doses of any of the compositions described herein.

The invention will be further described in the following examples, which do not limit the scope of the invention described in the claims.

### EXAMPLES

### Example 1—An Exemplary Composition

A composition can be prepared with the composition shown in Table 1 below.

TABLE 1
An exemplary composition.

| Zeolite | 100-3,000 | mg |
|---|---|---|
| Riboflavin | 1-2,000 | mg |
| Lactulose | 1-15 | g |
| An antibiotic (e.g., neomycin, | 5-2,000 | mg |
| metronidazole, and/or rifaxamin) | | |
| An ammonia sequestering agent | 1-5 | g |
| (e.g., L-ornithine-L-aspartate, L- | | |
| ornithine-α-ketoglutarate, and L- | | |
| ornithine-phenylacetate) | | |
| An intestinal deodorizer (e.g., | 10-2,000 | mg |

| | |
|---|---|
| charcoal and/or copper | |
| chlorophyllin) | |
| A probiotic (e.g., Lactobacillus | 1 × 10⁷ to 1 × 10¹³ CFUs |
| spp., Bifidobacterium spp., and | of each of Lactobacillus |
| Streptococcus thermophiles spp). | spp., Bifidobacterium |
| | spp., and Streptococcus |
| | thermophiles spp. |

### Example 2—An Exemplary Composition

A composition can be prepared with the composition shown in Table 2 below.

TABLE 2
An exemplary composition.

| Zeolite | 100-3,000 | mg |
|---|---|---|
| Riboflavin | 10-1,000 | mg |
| Lactulose | 5-15 | g |
| An antibiotic (e.g., neomycin, | 5-2,000 | mg |
| metronidazole, and/or rifaxamin) | | |
| An ammonia sequestering agent | 1-5 | g |
| (e.g., L-ornithine-L-aspartate, L- | | |
| ornithine-α-ketoglutarate, and L- | | |
| ornithine-phenylacetate) | | |
| An intestinal deodorizer (e.g., | 10-2,000 | mg |

| | |
|---|---|
| charcoal and/or copper | |
| chlorophyllin) | |
| A probiotic (e.g., Lactobacillus | 1 × 10⁷ to 1 × 10¹³ |
| spp., Bifidobacterium spp., and | CFUs of each of |
| Streptococcus thermophiles spp). | Lactobacillus spp., |
| | Bifidobacterium spp., |
| | and Streptococcus |
| | thermophiles spp. |

### Example 3—An Exemplary Composition

A composition can be prepared with the composition shown in Table 3 below.

TABLE 3
An exemplary composition.

| Zeolite | 500-1,500 | mg |
|---|---|---|
| Riboflavin | 10-1,000 | mg |
| Lactulose | 5-15 | g |
| An antibiotic (e.g., neomycin, | 5-2,000 | mg |
| metronidazole, and/or rifaxamin) | | |
| An ammonia sequestering agent | 1-5 | g |
| (e.g., L-ornithine-L-aspartate, L- | | |
| ornithine-α-ketoglutarate, and L- | | |
| ornithine-phenylacetate) | | |
| An intestinal deodorizer (e.g., | 10-2,000 | mg |

| | |
|---|---|
| charcoal and/or copper | |
| chlorophyllin) | |
| A probiotic (e.g., Lactobacillus | 1 × 10⁷ to 1 × 10¹³ CFUs of |
| spp., Bifidobacterium spp., and | each of Lactobacillus spp., |
| Streptococcus thermophiles spp). | Bifidobacterium spp., and |
| | Streptococcus thermophiles |
| | spp. |

### Example 4—An Exemplary Composition

A composition can be prepared with the composition shown in Table 4 below.

TABLE 4
An exemplary composition.

| Zeolite | 100-3,000 | mg |
|---|---|---|
| Riboflavin | 1-2,000 | mg |
| Lactulose | 1-15 | g |
| An antibiotic (e.g., neomycin, | 5-2,000 | mg |
| metronidazole, and/or rifaxamin) | | |
| An ammonia sequestering agent | 1-5 | g |
| (e.g., L-ornithine-L-aspartate, L- | | |
| ornithine-α-ketoglutarate, and L- | | |
| ornithine-phenylacetate) | | |
| An intestinal deodorizer (e.g., | 10-2,000 | mg |
| charcoal and/or copper | | |
| chlorophyllin) | | |
| A probiotic (e.g., Lactobacillus | 1 × 10⁷ to 1 × 10¹³ CFUs | |
| spp., Bifidobacterium spp., and | of each of Lactobacillus | |
| Streptococcus thermophiles spp). | spp., Bifidobacterium | |
| | spp., and Streptococcus | |
| | thermophiles spp. | |
| Valerenic acid | 1-50 | mg |
| Carnosic acid | 1-50 | mg |
| Vitamin E | 10-2,000 | IU |

### Example 5—An Exemplary Composition

A composition can be prepared with the composition shown in Table 5 below.

TABLE 5
An exemplary composition.

| Zeolite | 100-3,000 | mg |
|---|---|---|
| Riboflavin | 10-1,000 | mg |
| Lactulose | 5-15 | g |
| An antibiotic (e.g., neomycin, | 5-2,000 | mg |
| metronidazole, and/or rifaxamin) | | |
| An ammonia sequestering agent | 1-5 | g |
| (e.g., L-ornithine-L-aspartate, L- | | |
| ornithine-α-ketoglutarate, and L- | | |
| ornithine-phenylacetate) | | |
| An intestinal deodorizer (e.g., | 10-2,000 | mg |
| charcoal and/or copper | | |
| chlorophyllin) | | |
| A probiotic (e.g., Lactobacillus | 1 × 10⁷ to 1 × 10¹³ CFUs of | |
| spp., Bifidobacterium spp., and | each of Lactobacillus spp., | |
| Streptococcus thermophiles spp). | Bifidobacterium spp., and | |
| | Streptococcus thermophiles | |
| | spp. | |
| Valerenic acid | 5-15 | mg |
| Carnosic acid | 5-15 | mg |
| Vitamin E | 25-500 | IU |

### Example 6—An Exemplary Composition

A composition can be prepared with the composition shown in Table 6 below.

TABLE 6
An exemplary composition.

| Zeolite | 500-1,500 | mg |
|---|---|---|
| Riboflavin | 10-1,000 | mg |
| Lactulose | 5-15 | g |
| An antibiotic (e.g., neomycin, | 5-2,000 | mg |
| metronidazole, and/or rifaxamin) | | |
| An ammonia sequestering agent | 1-5 | g |
| (e.g., L-ornithine-L-aspartate, L- | | |
| ornithine-α-ketoglutarate, and L- | | |
| ornithine-phenylacetate) | | |
| An intestinal deodorizer (e.g., | 10-2,000 | mg |
| charcoal and/or copper | | |
| chlorophyllin) | | |
| A probiotic (e.g., Lactobacillus | 1 × 10⁷ to 1 × 10¹³ CFUs of | |
| spp., Bifidobacterium spp., and | each of Lactobacillus spp., | |
| Streptococcus thermophiles spp). | Bifidobacterium spp., and | |
| | Streptococcus thermophiles | |
| | spp. | |
| Valerenic acid | 5-15 | mg |
| Carnosic acid | 5-15 | mg |
| Vitamin E | 25-500 | IU |

### Example 7—An Exemplary Composition

A composition can be prepared with the composition shown in Table 7 below.

TABLE 7
An exemplary
composition.

| | | |
|---|---|---|
| Zeolite | 100-3,000 | mg |
| Valerenic acid | 1-50 | mg |
| Carnosic acid | 1-50 | mg |
| Vitamin E | 10-2,000 | IU |

### Example 8—An Exemplary Composition

A composition can be prepared with the composition shown in Table 8 below.

TABLE 8
An exemplary
composition.

| | | |
|---|---|---|
| Zeolite | 100-3,000 | mg |
| Valerenic acid | 5-15 | mg |
| Carnosic acid | 5-15 | mg |
| Vitamin E | 25-500 | IU |

### Example 9—An Exemplary Composition

A composition can be prepared with the composition shown in Table 9 below.

TABLE 9
An exemplary
composition.

| | | |
|---|---|---|
| Zeolite | 500-1,500 | mg |
| Valerenic acid | 5-15 | mg |
| Carnosic acid | 5-15 | mg |
| Vitamin E | 25-500 | IU |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating trimethylaminuria (TMAU) in a subject in need thereof comprising orally administering to the subject a therapeutically effective amount of a composition comprising a zeolite.

2. A method of decreasing a level of trimethylamine or decreasing a level of trimethylamine precursor in one or more of urine, sweat, reproductive fluid, saliva, blood, plasma, serum, and breath in a subject in need thereof comprising orally administering to the subject a therapeutically effective amount of a composition comprising a zeolite.

3. (canceled)

4. A method of increasing a trimethylamine N-oxide ratio in one or more of urine, sweat, reproductive fluid, saliva, blood, plasma, serum, and breath in a subject in need thereof comprising orally administering to the subject a therapeutically effective amount of a composition comprising a zeolite, where the trimethylamine N-oxide ratio is Formula I:
[(level of trimethylamine N-oxide)/(level of trimethylamine N-oxide+level of trimethylamine)]×100%.

5. A method of decreasing a trimethylamine ratio in one or more of urine, sweat, reproductive fluid, saliva, blood, serum, plasma, and breath in a subject in need thereof comprising orally administering to the subject a therapeutically effective amount of a composition comprising a zeolite, where the trimethylamine ratio is Formula II:
[(level of trimethylamine)/(level of trimethylamine N-oxide+level of trimethylamine)]×100%.

6. The method of claim 1, wherein the subject has been diagnosed as having primary TMAU (TMAU1).

7. The method of claim 6, wherein the subject has been determined to have a dysregulating mutation in a flavin-containing monooxygenase 3 (FMO3) gene.

8. The method of claim 1, wherein the subject has been diagnosed as having secondary TMAU (TMAU2).

9. The method of claim 1, wherein the subject does not have a urinary tract infection, bacterial vaginosis, cervical cancer, and advanced liver or kidney disease.

10. The method of claim 1, wherein the subject has been identified with enzymatic defects associated with TMAU (TMAU2).

11. The method of claim 1, wherein the composition is a zeolite suspension or a zeolite slurry comprising one or more solutions.

12.-13. (canceled)

14. The method of claim 10, wherein about 0.1% w/w to about 25% w/w of the composition is represented by the zeolite.

15. The method of claim 10, wherein the one or more solution or combination thereof, the zeolite suspension, or the zeolite slurry has a pH of about 9.0 to about 13.0.

16.-17. (canceled)

18. The method of claim 1, wherein the zeolite has the following formula:
M_{a/n}(AlO₂)ₐ(SiO₂)_{b}.wH₂O,
where:
w is the number of water molecules per unit cell varying from 1 to 10; and
a and b are total numbers of tetrahedra of Al and Si, respectively, wherein the ratio of b/a ranges from about 0.5 to about 5.

19.-22. (canceled)

23. The method of claim 1, wherein the zeolite has a pore size of about 2.0 angstroms to about 5.5 angstroms.

24.-25. (canceled)

26. The method of claim 1, wherein the composition is a pharmaceutical composition.

27. The method of claim 1, wherein the zeolite is a natural zeolite.

28. The method of claim 1, wherein the zeolite is a synthetic zeolite.

29. The method of claim 1, wherein the zeolite is selected from the group consisting of a Type 3A zeolite, a Type 4A zeolite, a Type 5A zeolite, a Type 13X zeolite, and combinations thereof.

30. (canceled)

31. The method of claim 1, wherein the composition comprises about 100 mg to about 3,000 mg of the zeolite.

32. (canceled)

33. The method of claim 1, wherein the zeolite is the only active ingredient in the composition.

34. The method of claim 1, wherein the composition further comprises one or more additional active ingredients selected from the group consisting of: riboflavin, a colonic acidifier, an ammonia reducer, an ammonia sequestering agent, an intestinal deodorizer, and a probiotic.

35.-52. (canceled)

53. The method of claim 1, wherein the composition is provided in an oral dosage form.

54. The method of claim 53, wherein the oral dosage form is a tablet or a capsule.

55. (canceled)

56. A capsule comprising a therapeutically effective amount of a zeolite and a therapeutically effective amount of one or more additional active ingredients selected from the group consisting of: riboflavin, a colonic acidifier, an ammonia reducer, an ammonia sequestering agent, an intestinal deodorizer, and a probiotic.

57.-58. (canceled)
